(19) United States
(12) Patent Application Publication
(10) Pub. No.: US 2026/0199309 A1
(43) Pub. Date: Jul. 16, 2026

(54) COMPOUNDS FOR THE TREATMENT OF CENTRA NERVOUS SYSTEM DISORDERS

(71) Applicant: Autifony Therapeutics Limited, Hertfordshire (GB)

(72) Inventors: Giuseppe Alvaro, Hertfordshire (GB); Agostino Marasco, Hertfordshire (GB)

(21) Appl. No.: 19/136,262

(22) PCT Filed: Dec. 6, 2023
(86) PCT No.: PCT/GB2023/053142

§ 371 Date: Jun. 5, 2025

(30) Foreign Application Priority Data
Dec. 6, 2022 (EP) 22211694.9

(51) Int. Cl. A61K 31/4439; C07D 401/04; C07D 405/14

(52) U.S. Cl. CPC A61K 31/4439; C07D 401/04; C07D 405/14

(57) ABSTRACT

The present invention provides Kv3 modulator compounds of the formula (I): and related aspects.

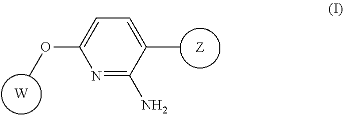

### TECHNICAL FIELD

This invention relates to novel compounds, pharmaceutical compositions containing them and their use as medicaments, in particular in the prophylaxis or treatment of progressive myoclonic epilepsy, including PME associated with mutations in the KCNC1 gene, hearing disorders, including hearing loss and tinnitus, as well as Fragile X syndrome, schizophrenia, substance abuse disorders, and pain.

### BACKGROUND TO THE INVENTION

The Kv3 voltage-gated potassium channel family includes four members, Kv3.1, Kv3.2, Kv3.3, and Kv3.4. Kv3 channels are activated by depolarisation of the plasma membrane to voltages more positive than −20 mV; furthermore, the channels deactivate rapidly upon repolarisation of the membrane. These biophysical properties ensure that the channels open towards the peak of the depolarising phase of the neuronal action potential to initiate repolarisation. Rapid termination of the action potential mediated by Kv3 channels allows the neuron to recover more quickly to reach sub-threshold membrane potentials from which further action potentials can be triggered. As a result, the presence of Kv3 channels in certain neurons contributes to their ability to fire at high frequencies (Rudy et al., 2001). Kv3 subtypes are predominant in the CNS, although Kv3.1, Kv3.3 and Kv3.4 channels are also found in spinal cord (Brooke et al., 2002; 2004; 2006). Kv3 channel subtypes are differentially expressed by sub-classes of interneurons in cortical and hippocampal brain areas (e.g. Chow et al., 1999; Martina et al., 1998; McDonald et al., 2006; Chang et al., 2007), in the thalamus (e.g. Kasten et al., 2007), cerebellum (e.g. Sacco et al., 2006; Puente et al., 2010), and auditory brain stem nuclei (Li et al., 2001).

Characterisation of mice in which one or more of the Kv3 subtypes has been deleted shows that the absence of Kv3.1 gives rise to increased locomotor activity, altered electroencephalographic activity, and a fragmented sleep pattern (Joho et al., 1999). The deletion of Kv3.2 leads to a reduction in seizure threshold and altered cortical electroencephalographic activity (Lau et al., 2000). Deletion of Kv3.3 is associated with mild ataxia and motor deficits (McMahon et al., 2004). Double deletion of Kv3.1 and Kv3.3 gives rise to a severe phenotype characterised by spontaneous seizures, ataxia, and an increased sensitivity to the effects of ethanol (Espinosa et al., 2001; Espinosa et al., 2008). A spontaneous mutation in the Kv3.1 gene (KCNC1) in humans causes progressive myoclonic epilepsy (Muona et al., 2014). Mutation of the Kv3.3 gene (KCNC3) in humans is associated with spinocerebellar ataxia (SCA13) (Figueroa et al., 2010).

Bipolar disorder, schizophrenia, are serious disorders of the central nervous system that have been associated with reduced function of parvalbumin-positive inhibitory interneurons in corticolimbic brain circuits (Reynolds et al., 2004; Benes et al., 2008; Brambilla et al., 2003; Aroniadou-Anderjaska et al., 2007; Ben-Ari, 2006). Parvalbumin positive basket cells express Kv3 channels which allow these neurons to fire at high frequency to provide fast feedback inhibition within local circuits (Markram et al., 2004). Accurate timing of this inhibitory feedback is necessary to sustain network synchronisation, for example, in the generation of gamma frequency field potential oscillations that have been associated with cognitive function (Fisahn et al., 2005; Engel et al., 2001). A reduction in gamma oscillations has been observed in patients with schizophrenia (Spencer et al., 2004), and evidence suggests reduced expression of Kv3.1, but not Kv3.2 in the dorsolateral prefrontal cortex of patients with schizophrenia (Yanagi et al., 2014). Positive modulators of Kv3 channels enhance the firing of parvalbumin positive interneurons (Rosato-Siri et al., 2015; Boddum et al., 2017) leading to increased gamma oscillations (Andrade-Talavera et al., 2020) and rescue deficits in cognitive and social behaviours in animal models (Leger et al., 2015). Furthermore, Kv3 positive modulators reduced abonormal behaviours in a mouse model of bipolar disorder (Parekh et al., 2017)

Fragile X syndrome is a paediatric developmental disorder with autistic features that has also been linked to dysfunction of parvalbumin positive interneurons (e.g. Pirbhoy et al., 2020) and alteration of Kv3.1 channel expression (Darnell et al., 2001; Strumbos et al., 2010). Kv3 channel modulators have been shown to rescue deficits in auditory brainstem function in vitro and in vivo in a mouse model of Fragile X syndrome (EI-Hassar et al., 2019).

Voltage-gated ion channels of the Kv3 family are expressed at high levels in auditory brainstem nuclei (Li et al., 2001) where they permit the fast firing of neurons that transmit auditory information from the cochlear to higher brain regions. Phosphorylation of Kv3.1 and Kv3.3 channels in auditory brainstem neurons is suggested to contribute to the rapid physiological adaptation to sound levels that may play a protective role during exposure to noise (Desai et al., 2008; Song et al., 2005). Loss of Kv3.1 channel expression in central auditory neurons is observed in hearing impaired mice (von Hehn et al., 2004); furthermore, a decline in Kv3.1 expression may be associated with loss of hearing in aged mice (Jung et al. 2005), and loss of Kv3 channel function may also follow noise-trauma induced hearing loss (Pilati et al., 2012). Furthermore, pathological plasticity of auditory brainstem networks is likely to contribute to symptoms that are experienced by many people suffering from hearing loss of different types. Recent studies have shown that regulation of Kv3.1 channel function and expression has a major role in controlling auditory neuron excitability (Kaczmarek et al., 2005; Anderson et al., 2018; Glait et al., 2018; Olsen et al., 2018, Chambers et al., 2017), suggesting that this mechanism could account for some of the plastic changes that give rise to hearing-related disorders such as tinnitus.

Recently, Kv3.4 channels have become a target of interest for the treatment of chronic pain. Kv3.4 channels are expressed on neurons of the dorsal root ganglia (Ritter et al., 2012; Chien et al., 2007), where they are predominantly expressed on sensory C-fibres (Chien et al., 2007). Kv3 channels are also expressed by specific subsets of neurons in the spinal cord. Specifically, Kv3.1b (Deuchars et al., 2001; Brooke et al., 2002), Kv3.3 (Brooke et al., 2006), and Kv3.4 subunits (Brooke et al., 2004) have been identified in rodent spinal cord, although not always in association with circuits involved with sensory processing. It is likely that Kv3 channels shape the firing properties of spinal cord neurons, including motoneurons.

In addition recent studies showed the Kv3.4 channels expressed in DRG nociceptors have a significant impact on glutamatergic synaptic transmission (Muqeem et al., 2018). animal model data suggest a down-regulation of Kv3.4 channel surface expression in DRG neurons following spinal cord injury associated with hypersensitivity to painful stimuli (Ritter et al., 2015; Zemel et al., 2017; Zemel et al., 2018). Similarly, it has been observed that there is a down-regulation of Kv3.4 expression in DRGs of rodents following spinal cord ligation (Chien et al., 2007). This latter study also showed that intrathecal administration to rats of an antisense oligonucleotide to suppress the expression of Kv3.4 led to hypersensitivity to mechanical stimuli. It has been shown that Kv3.4 channel inactivation could be influenced by protein kinase C-dependent phosphorylation of the channels, and that this physiological mechanism might allow DRG neurons to alter their firing characteristics in response to painful stimuli (Ritter et al., 2012). These studies suggest a causal relationship between the emergence of mechanical allodynia and reduced Kv3.4 channel expression or function. No evaluation of Kv3.1, Kv3.2, or Kv3.3 expression in SC or DRG neurons was conducted in any of these studies, and expression of these subtypes has not been explicitly demonstrated on DRG neurons (although as mentioned above, they are abundant within specific regions of the spinal cord). The in vivo studies reported above provide a rationale for modulation of Kv3.4 as a novel approach to the treatment of certain neuropathic pain states.

Patent applications WO2011/069951, WO2012/076877, WO2012/168710, WO2013/175215, WO2013/083994, WO2013/182850, WO2017/103604, WO2018/020263, WO2018/109484, WO2020/079422, WO2021/156584 and WO2023/017263 disclose compounds which are modulators of Kv3.1 and Kv3.2. Further, the utility of such compounds is demonstrated in animal models of seizure, hyperactivity, sleep disorders, psychosis, hearing disorders and bipolar disorders.

Patent application WO2013/182851 discloses modulation of Kv3.3 channels by certain compounds.

Patent application WO2013/175211 discloses that modulation of Kv3.1, Kv3.2 and/or Kv3.3 channels has been found to be beneficial in preventing or limiting the establishment of a permanent hearing loss resulting from acute noise exposure. The benefits of such prevention may be observed even after administration of the Kv3.1, Kv3.2 and/or Kv3.3 modulator has ceased.

Patent application WO2017/098254 discloses that modulation of Kv3.1, Kv3.2 and/or Kv3.3 channels has been found to be beneficial in the prophylaxis or treatment of pain, in particular neuropathic or inflammatory pain.

Patent applications WO2019/222816, WO2020/000065, WO2020/089262, WO2020/216919, WO2020/216920 and WO2021/214090 describe compounds which are said to activate Kv3 potassium channels.

There remains a need for the identification of alternative modulators of Kv3.1, Kv3.2, Kv3.3 and/or or Kv3.4 channels, in particular modulators of Kv3.1 and/or Kv3.2. Such modulators may demonstrate high in vivo potency, channel selectivity, an improved safety profile, or desirable pharmacokinetic parameters, for example high brain availability and/or low clearance rate that reduces the dose required for therapeutic effect in vivo. Alternative modulators may provide a benefit through having distinct metabolites from known modulators.

Certain compounds may demonstrate properties more suitable for peripheral availability and associated indications.

Compounds which have balanced Kv3.1, Kv3.2, Kv3.3 and/or Kv3.4 modulatory properties may be desirable e.g. compounds with modulate Kv3.1 and Kv3.2 to the same, or a similar extent. For certain therapeutic indications, there is also a need to identify compounds with a different modulatory effect on Kv3.1, Kv3.2, Kv3.3 and/or Kv3.4 channels, for example, compounds that alter the kinetics of channel gating or channel inactivation, and which may behave in vivo as negative modulators of the channels.

### SUMMARY OF THE INVENTION

The present invention provides a compound of formula (I):

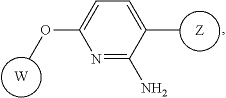

wherein
W is group (Wa), group (Wb) or group (Wc):
   wherein group (Wa) and group (Wb) are:

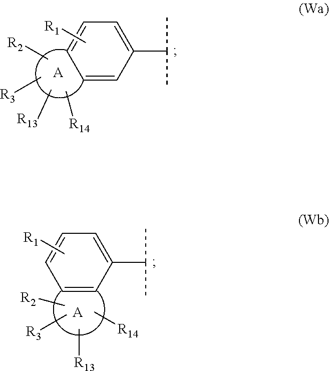

wherein:
   R₁ is H, C₁₋₄alkyl, halo, haloC₁₋₄alkyl, CN, C₁₋₄alkoxy or haloC₁₋₄alkoxy;
   R₂ is H, C₁₋₄alkyl, C₃₋₅ spiro carbocyclyl, haloC₁₋₄alkyl or halo;
   R₃ is H, C₁₋₄alkyl, haloC₁₋₄alkyl, halo; or R₃ is absent;
   R₁₃ is H, C₁₋₄alkyl, haloC₁₋₄alkyl, halo; or R₁₃ is absent;
   R₁₄ is H, C₁₋₄alkyl, haloC₁₋₄alkyl, halo; or R₁₄ is absent;
   A is a 5 or 6 membered saturated or unsaturated heterocycle, with at least one O atom; which heterocycle is optionally fused with a cyclopropyl group, or a cyclobutyl group, or a cyclopentyl group to form a tricycle when considered together with the phenyl;
   wherein R₂ and R₃ may be attached to the same or a different ring atom; R₂ may be attached to a fused ring atom; and wherein R₁₃ and R₁₄ may be attached to the same or a different ring atom;
wherein group (Wc) is:

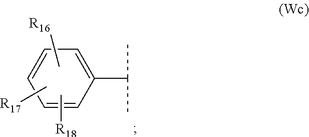

wherein:
   R₁₆ is halo, C₁₋₄alkyl, C₁₋₄alkoxy, haloC₁₋₄alkyl, haloC₁₋₄alkoxy or CN;
   R₁₇ is H, halo, CN, C₁₋₄alkyl, C₁₋₄alkoxy or haloC₁₋₄alkoxy;
   R₁₈ is H, halo, CN, C₁₋₄alkyl or C₁₋₄alkoxy;
Z is group (Za):

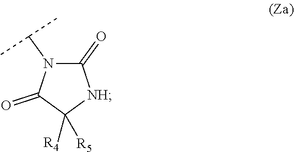

wherein:
   R₄ is H or C₁₋₄ alkyl;
   R₅ is H or C₁₋₄ alkyl; or
   R₄ and R₅ can be fused to form a C₃₋₅ spiro carbocyclyl;
      or a salt and/or solvate and/or derivative thereof.

A compound of formula (I) may be provided in the form of a pharmaceutically acceptable salt and/or solvate thereof and/or derivative thereof. In one embodiment, the compound of formula (I) is provided in the form of a pharmaceutically acceptable salt. In one embodiment, the compound of formula (I) is provided in the form of a solvate. In one embodiment, the compound of formula (I) is provided in the form of a pharmaceutically acceptable solvate. In one embodiment, the compound of formula (I) is provided in the form of a pharmaceutically acceptable derivative. In one embodiment, the compound of formula (I) is provided in the form of the free base. In one embodiment, the compound of formula (I) is provided in the form of a salt. In one embodiment, the compound of formula (I) is provided in the form of a derivative.

The compounds of formula (I) may be used as medicaments, in particular for use in the prophylaxis or treatment of progressive myoclonic epilepsy, including PME associated with mutations in the KCNC1 gene, hearing disorders, including hearing loss and tinnitus, as well as Fragile X syndrome, schizophrenia, substance abuse disorders, or pain.

Further, there is provided a method for the prophylaxis or treatment of progressive myoclonic epilepsy, including PME associated with mutations in the KCNC1 gene, hearing disorders, including hearing loss and tinnitus, as well as Fragile X syndrome, schizophrenia, substance abuse disorders, or pain in a subject, the method comprising administering a compound of formula (I).

Compounds of formula (I) may be used in the manufacture of a medicament for the prophylaxis or treatment of progressive myoclonic epilepsy, including PME associated with mutations in the KCNC1 gene, hearing disorders, including hearing loss and tinnitus, as well as Fragile X syndrome, schizophrenia, substance abuse disorders, or pain.

Also provided are pharmaceutical compositions containing a compound of formula (I) and a pharmaceutically acceptable carrier or excipient.

Also provided are pharmaceutical compositions containing a compound of formula (I) or a pharmaceutically acceptable salt, and/or solvate and/or derivative thereof and a pharmaceutically acceptable carrier or excipient.

Also provided are processes for preparing compounds of formula (I) and novel intermediates of use in the preparation of compounds of formula (I).

Also provided are processes for preparing compounds of formula (I) or a salt and/or solvate and/or derivative thereof, preferably a pharmaceutically acceptable salt and/or solvate and/or derivative thereof, and novel intermediates of use in the preparation of compounds of formula (I) or a salt and/or solvate and/or derivative thereof, preferably a pharmaceutically acceptable salt and/or solvate and/or derivative thereof.

Additionally provided are prodrug derivatives of the compounds of formula (I).

### DETAILED DESCRIPTION OF THE INVENTION

The present invention provides a compound of formula (I):

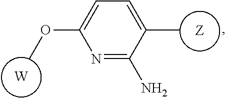

wherein
W is group (Wa), group (Wb) or group (Wc):
   wherein group (Wa) and group (Wb) are:

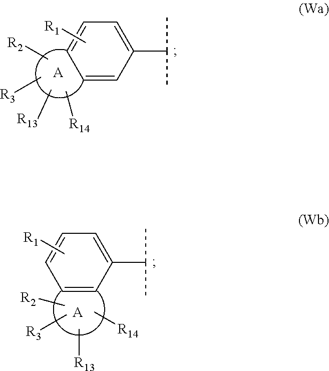

wherein:
   R₁ is H, C₁₋₄alkyl, halo, haloC₁₋₄alkyl, CN, C₁₋₄alkoxy or haloC₁₋₄alkoxy;
   R₂ is H, C₁₋₄alkyl, C₃₋₅ spiro carbocyclyl, haloC₁₋₄alkyl or halo;
   R₃ is H, C₁₋₄alkyl, haloC₁₋₄alkyl, halo; or R₃ is absent;
   R₁₃ is H, C₁₋₄alkyl, haloC₁₋₄alkyl, halo; or R₁₃ is absent;
   R₁₄ is H, C₁₋₄alkyl, haloC₁₋₄alkyl, halo; or R₁₄ is absent;
   A is a 5 or 6 membered saturated or unsaturated heterocycle, with at least one O atom; which heterocycle is optionally fused with a cyclopropyl group, or a cyclobutyl group, or a cyclopentyl group to form a tricycle when considered together with the phenyl;
   wherein R₂ and R₃ may be attached to the same or a different ring atom; R₂ may be attached to a fused ring atom; and wherein R₁₃ and R₁₄ may be attached to the same or a different ring atom;
wherein group (Wc) is:

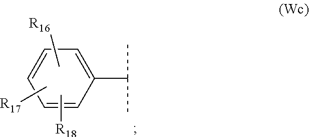

wherein:
   R₁₆ is halo, C₁₋₄alkyl, C₁₋₄alkoxy, haloC₁₋₄alkyl, haloC₁₋₄alkoxy or CN;
   R₁₇ is H, halo, CN, C₁₋₄alkyl, C₁₋₄alkoxy or haloC₁₋₄alkoxy;
   R₁, is H, halo, CN, C₁₋₄alkyl or C₁₋₄alkoxy;
Z is group (Za):

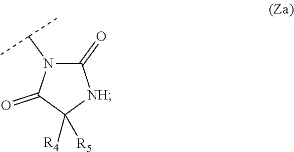

wherein:
   R₄ is H or C₁₋₄ alkyl;
   R₅ is H or C₁₋₄ alkyl; or
   R₄ and R₅ can be fused to form a C₃₋₅ spiro carbocyclyl.

The invention further provides a pharmaceutically acceptable salt of a compound of formula (I). The invention further provides a solvate of a compound of formula (I). The invention further provides a pharmaceutically acceptable salt and/or solvate of a compound of formula (I).

The invention further provides a salt of a compound of formula (I). The invention further provides a derivative of a compound of formula (I). The invention further provides a pharmaceutically acceptable derivative of a compound of formula (I). The invention further provides a compound of formula (I) as the free base. The invention further provides a pharmaceutically acceptable solvate of a compound of formula (I).

The compounds of formula (I), and salts and/or solvates and/or derivatives thereof, may be referred to herein as ‘compounds of the invention’.

Embodiments set out below relating to relative stereochemistry and the nature of groups, including A, W, (Wa), (Wb), (Wc), Z, (Za), R₁, R₂, R₃, R₄, R₅, R₁₃, R₁₄, R₁₆, R₁₇, R₁₈, R₂₀, R₂₁, R₂₂, R₂₃ and R₂₄ are envisaged as being independently, fully combinable with one another to form further embodiments of the invention. Such embodiments apply equally to intermediates which may be of use in the synthesis of a compound of formula (I) e.g. compounds of formulae (II), (Ill), (IV), (V), (VI) and (VII)

It will be appreciated that the selection of combinations of functional groups for compounds of formula (I) should result in chemically sensible structures. For example, the positioning of heteroatoms in inappropriate proximity may cause instability.

In one embodiment, R₄ is C₁₋₄ alkyl, such as methyl or ethyl. In one embodiment, R₅ is C₁₋₄ alkyl, such as methyl or ethyl.

In one embodiment, R₄ is methyl, ethyl, isopropyl or tert-butyl, in particular methyl. In one embodiment, R₅ is C₁₋₄ alkyl such as methyl, ethyl, isopropyl or tert-butyl. In one embodiment R₄ is H. In one embodiment R₅ is H. In one embodiment, R₄ and R₅ are fused to form a C₃₋₄ spiro carbocyclyl.

Suitably R₄ is H, methyl or ethyl, and R₅ is independently H, methyl or ethyl.

In one embodiment, R₄ is C₁₋₄ alkyl such as methyl, ethyl, isopropyl or tert-butyl, and R₅ is H, in particular R₄ is ethyl and R₅ is H. In one embodiment, R₄ is C₁₋₄ alkyl such as methyl, ethyl, isopropyl or tert-butyl, and R₅ is C₁₋₄ alkyl such as methyl, ethyl, isopropyl or tert-butyl. In this embodiment, R₄ and R₅ may be the same or different. In one embodiment, R₄ and R₅ are both methyl. In one embodiment, R₄ and R₅ are both ethyl. In one embodiment, one of R₄ and R₅ is methyl and the remaining R₄ or R₅ is ethyl. In one embodiment R₄ is H and R₅ is H.

In embodiments wherein R₄ and R₅ are different, they may have the following stereochemical arrangement:

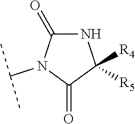

In this embodiment, for example, R₄ is C₁₋₄ alkyl such as methyl, ethyl, isopropyl or tert-butyl, especially methyl or ethyl, in particular ethyl, and R₅ is H. Alternatively, R₄ may be C₂₋₄ alkyl such as ethyl, isopropyl or tert-butyl, and R₅ is C₁₋₃ alkyl such as methyl, ethyl or isopropyl, but R₄ contains a greater number of carbon atoms than R₅.

In embodiments wherein R₄ and R₅ are different, they may alternatively have the following stereochemical arrangement:

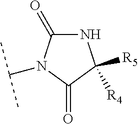

In this embodiment, for example, R₄ is C₁₋₄ alkyl such as methyl, ethyl, isopropyl or tert-butyl, especially methyl or ethyl, in particular ethyl, and R₅ is H. Alternatively, R₄ may be C₂₋₄ alkyl such as ethyl, isopropyl or tert-butyl, and R₅ is C₁₋₃ alkyl such as methyl, ethyl or isopropyl, but R₄ contains a greater number of carbon atoms than R₅.

Group W is group (Wa), group (Wb) or group (Wc). In one embodiment, group W is group (Wa). In one embodiment, group W is group (Wb). In one embodiment, group W is group (Wc). Groups (Wa) and (Wb) both contain ring A and bear substituents R₁, R₂, R₃, R₁₃ and R₁₄.

In one embodiment, ring A is a 5 membered saturated heterocycle, with at least one O atom; which heterocycle is optionally fused with a cyclopropyl group, or a cyclobutyl group, or a cyclopentyl group to form a tricycle when considered together with the phenyl. In one embodiment, ring A contains one heteroatom which is oxygen. In one embodiment, ring A contains two heteroatoms, e.g. two oxygen atoms or one oxygen atom and one nitrogen atom. In one embodiment ring A is dihydrofuran, isoxazole, dihydropyran, 1,3-dioxolane, 1,3-oxazine or dihydropyran. Suitably, ring A is dihydrofuran or dihydropyran, in particular dihydrofuran.

In one embodiment, ring A is selected from the group consisting of:

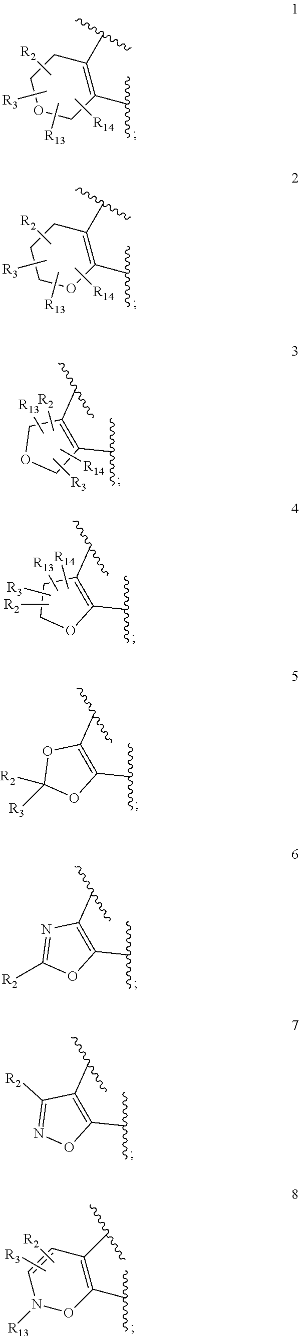

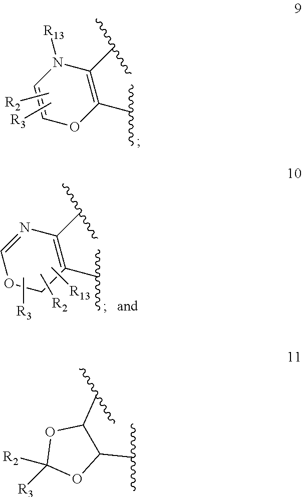

wherein

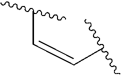 [SMILES: */C=C\*]

denotes a point at which ring A is fused to the phenyl ring.

In one embodiment, ring A is selected from the group consisting of:

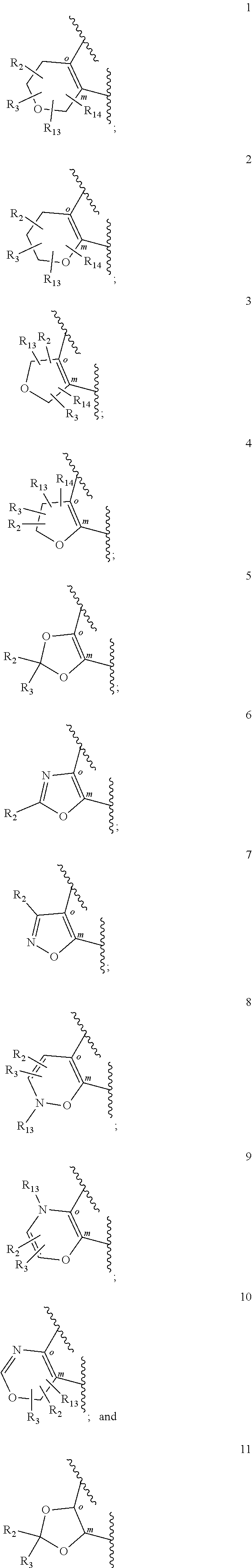

wherein

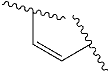 [SMILES: */C=C\*]

denotes a point at which ring A is fused to the phenyl ring, and “o” and “in” indicate the ortho- and meta-positions of the phenyl ring to which group A is fused.

In one embodiment, ring A is selected from the group consisting of:

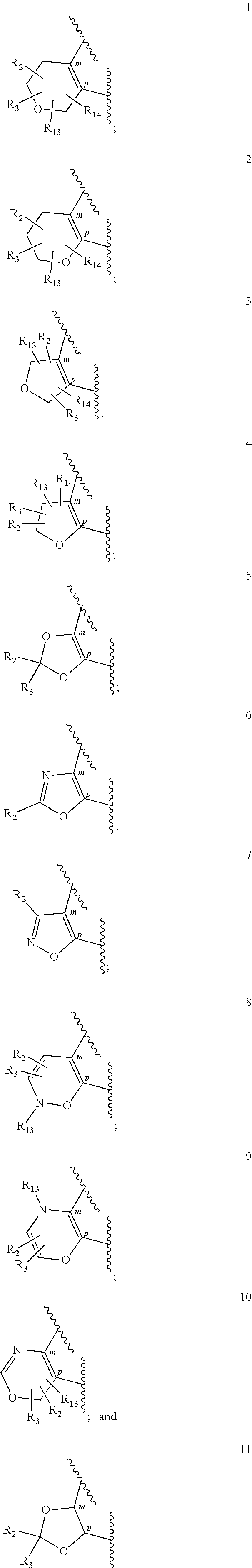

wherein

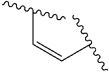 [SMILES: */C=C\*]

denotes a point at which ring A is fused to the phenyl ring, wherein “m” and “p” indicate the meta- and para-positions of the phenyl ring to which group A is fused.

Suitably, ring A is:

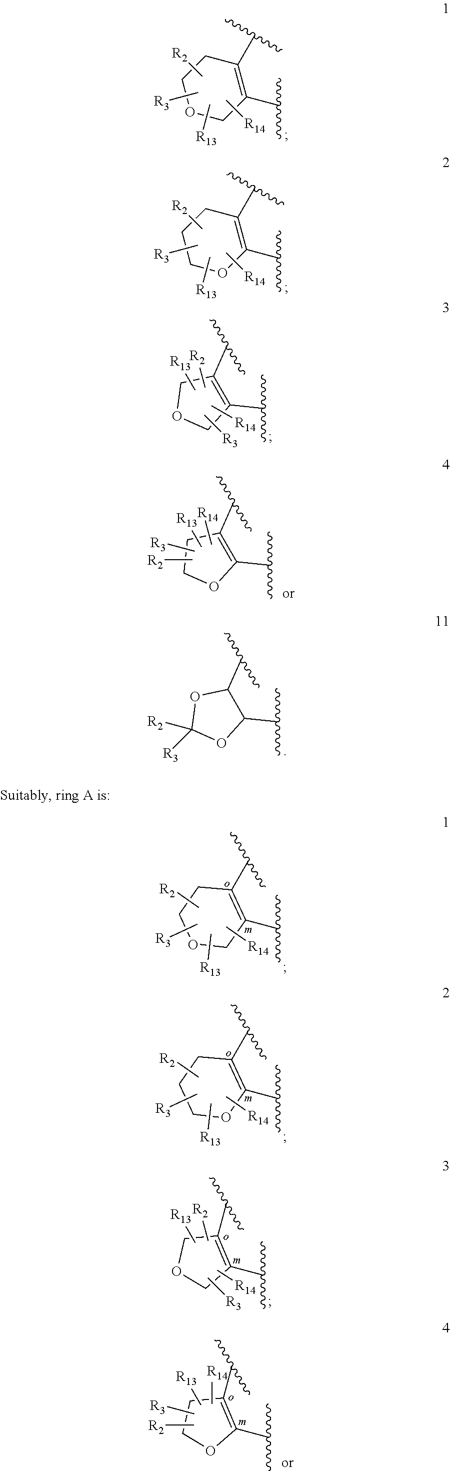

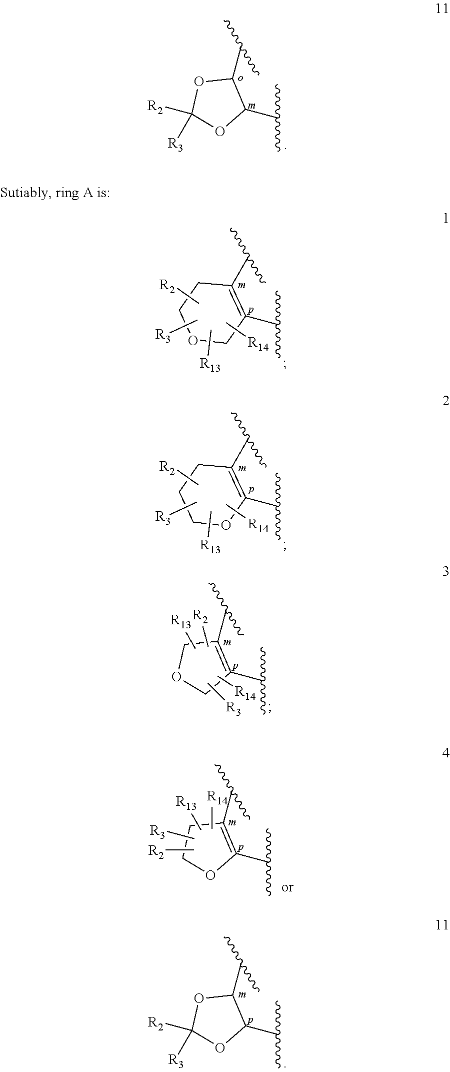

In particular, ring A is

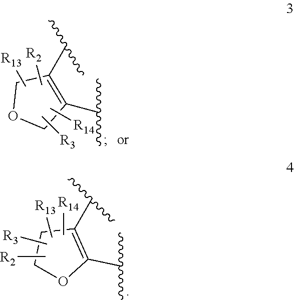

In particular, ring A is

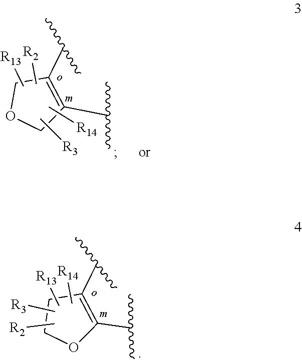

In particular, ring A is

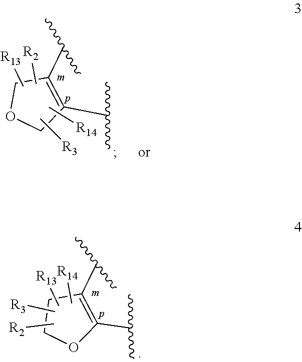

In one embodiment, when ring A is a 5 membered heterocycle containing one heteroatom which is oxygen, wherein suitably the oxygen atom is located at the phenolic position relative to the phenyl ring.

In one embodiment, R₁ is H, C₁₋₄alkyl, halo, haloC₁₋₄alkyl or CN, in particular C₁₋₄alkyl such as methyl. In one embodiment, R₁ is H, methyl or CN. In one embodiment R₁ is methyl. In one embodiment, R₁ is CN. In one embodiment R₁ is C₁₋₄alkoxy, in particular methoxy or ethoxy such as methoxy. In one embodiment R₁ is H.

In one embodiment, when W is group (Wb) R₁ is at the para position and is not H:

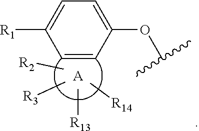

In one embodiment, when W is group (Wb) R₁ is at the meta position and is not H:

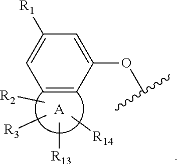

In one embodiment, when W is group (Wb) R₁ is at the ortho position and is not H:

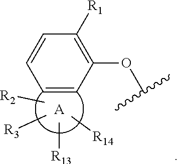

In one embodiment, R₂ is H, C₁₋₄alkyl, C₃₋₅spiro carbocyclyl or halo. In one embodiment, R₂ is C₁₋₄ alkyl such as methyl or ethyl or C₃₋₅spiro carbocyclyl such as C₃ spiro carbocycle. In one embodiment, R₂ is methyl. In one embodiment, R₂ is halo e.g. fluoro.

In one embodiment, R₃ is H, C₁₋₄alkyl, haloC₁₋₄alkyl or halo. In one embodiment, R₃ is C₁₋₄alkyl such as methyl. In another embodiment R₃ is halo e.g. fluoro. In another embodiment R₃ is absent.

In one embodiment, R₂ and R₃ are located on the same ring A atom.

In one embodiment, R₁₃ is H or is absent. Suitably, R₁₃ is absent.

In one embodiment, R₁₄ is H or is absent. Suitably, R₁₄ is absent.

In one embodiment, W is selected from the group consisting of:

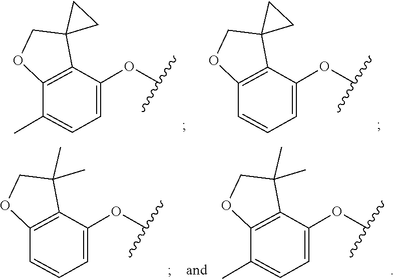

In one embodiment, W is

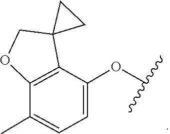 [SMILES: *Oc1ccc(C)c2c1C1(CC1)CO2]

In one embodiment, W is

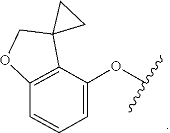 [SMILES: *Oc1cccc2c1C1(CC1)CO2]

In one embodiment, W is

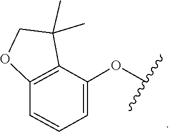 [SMILES: *Oc1cccc2c1C(C)(C)CO2]

In one embodiment, W is

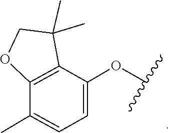 [SMILES: *Oc1ccc(C)c2c1C(C)(C)CO2]

Group (Wc) bears substituents R₁₆, R₁₇, and R₁₈.

In one embodiment, R₁₆ is not in the para position. In one embodiment, one of R₁₇ and R₁₃ is not H.

In one embodiment, R₁₆ is C₁₋₄alkyl, C₁₋₄alkoxy, haloC₁₋₄alkyl, haloC₁₋₄alkoxy or CN. In one embodiment, R₁₆ is C₁₋₄alkyl, C₁₋₄alkoxy, haloC₁₋₄alkyl or haloC₁₋₄alkoxy. In one embodiment, R₁₆ is C₁₋₄alkyl, C₁₋₄alkoxy or haloC₁₋₄alkoxy. In one embodiment, R₁₆ is halo, C₁₋₄alkyl or C₁₋₄alkoxy.

In one embodiment, R₁₆ is methyl, ethyl, propyl, butyl, cyclopropyl, chloro, fluoro, methoxy, ethoxy, propoxy, trifluoromethyl, trifluoromethoxy or CN. In one embodiment, R₁₆ is methoxy, trifluoromethoxy or iso-propyl. In one embodiment, R₁₆ is trifluoromethoxy. In one embodiment, R₁₆ is methoxy. In one embodiment, R₁₆ is iso-propyl.

In one embodiment, R₁₇ is H, halo, CN, C₁₋₄alkyl or C₁₋₄alkoxy. In one embodiment, R₁₇ is H, CN, C₁₋₄alkyl, C₁₋₄alkoxy or haloC₁₋₄alkoxy. In one embodiment, R₁₇ is C₁₋₄alkyl or C₁₋₄alkoxy. In one embodiment, R₁₇ is H, CN or C₁₋₄alkyl. In one embodiment, R₁₇ is H, CN or methyl. In one embodiment, R₁₇ is methyl, ethyl, propyl, butyl, cyclopropyl, chloro, fluoro, methoxy, ethoxy, propoxy, trifluoromethoxy or CN. In one embodiment, R₁₇ is H, methyl or CN. In one embodiment, R₁₇ is H. In one embodiment, R₁₇ is methyl. In one embodiment, R₁₇ is CN.

In one embodiment, R₁₈ is H.

In one embodiment, R₁₇ and R₁₈ are H. In this embodiment, R₁₆ is suitably at the ortho- or meta-position. In this embodiment, when R₁₆ is at the ortho position, it is suitably C₁₋₄ alkyl, for example methyl, ethyl, n-propyl, isopropyl, cyclopropyl, n-butyl or tert-butyl. In one embodiment, when R₁₆ is at the meta position, it is suitably C₁₋₄alkyl for example methyl, ethyl, isopropyl or cyclopropyl; C₁₋₄alkoxy for example methoxy or ethoxy; or haloC₁₋₄alkoxy for example trifluoromethoxy. In one embodiment, R₁₆ is methoxy at the meta-position and R₁₇ and R₁₃ are H. In one embodiment, R₁₆ is trifluoromethoxy at the meta-position and R₁₇ and R₁₈ are H.

In one embodiment, when the compound of formula (I) has syn configuration and R₁₆ is at the ortho position, R₁₆ is not tert-butyl.

In one embodiment, R₁₃ is H and R₁₇ is not H. When this is the case, in one embodiment one of R₁₆ or R₁₇ is at the ortho position. In this embodiment, the substituent at the ortho position is suitably C₁₋₄ alkyl, for example methyl, ethyl, n-propyl, isopropyl, cyclopropyl, n-butyl or tert-butyl. In another embodiment, one of R₁₆ and R₁₇ is at the ortho-position, and the other is at the meta-position. In this embodiment, the substituent at the ortho position is suitably C₁₋₄ alkyl, for example methyl, ethyl, n-propyl, isopropyl, cyclopropyl, n-butyl or tert-butyl and the substituent at the meta position is suitably C₁₋₄alkyl for example methyl, ethyl, isopropyl or cyclopropyl; C₁₋₄ alkoxy for example methoxy or ethoxy; or haloC₁₋₄alkoxy for example trifluoromethoxy. In one embodiment, one of R₁₆ and R₁₇ is at position 1-, and the other is at position 4-. In one embodiment, one of R₁₆ and R₁₇ is at the ortho-position, and the other is at the para-position. In this embodiment, the substituent in the para position is suitably CN, fluoro or methyl. In one embodiment, both of R₁, and R₁₇ are at the ortho-positions. In this embodiment, the substituents at the ortho position are suitably the same, and are suitably C₁₋₄ alkyl, for example methyl, ethyl, n-propyl, isopropyl, cyclopropyl, n-butyl or tert-butyl. In one embodiment, R₁₆ is iso-propyl at the meta-position and R₁₇ is CN at the para position. In one embodiment, R₁₆ is trifluoromethoxy at the meta-position and R₁₇ is methyl at the para-position.

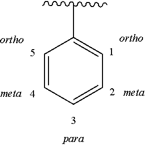 [SMILES: *c1ccccc1]

In one embodiment, (Wc is of formula (Wc-a):

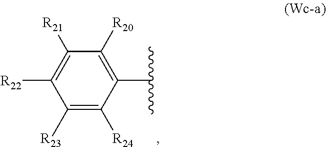

wherein
R₂₀ is H, C₁₋₄alkyl, haloC₁₋₄alkyl, or CN;
R₂₁ is H, halo, C₁₋₄alkyl, C₁₋₄alkoxy, haloC₁₋₄alkyl, haloC₁₋₄alkoxy or CN;
R₂₂ is H, halo, C₁₋₄alkyl, haloC₁₋₄alkyl, or CN;
R₂₃ is H, halo, C₁₋₄alkyl, C₁₋₄alkoxy, haloC₁₋₄alkyl or haloC₁₋₄alkoxy;
R₂₄ is H, C₁₋₄alkyl, haloC₁₋₄alkyl, or CN;
wherein at least 2, and suitably 3, of R₂₀, R₂₁, R₂₂, R₂₃ and R₂₄ are H,
and wherein at least one of R₂₀, R₂₁, R₂₂, R₂₃ and R₂₄ is not H.

It should be noted that compounds of formula (Wc-a) are intended to fall within the scope of formula (Wc), therefore the substituents R²⁰—R²⁴ are further limited in so far as the resulting phenyl group must fall within formula (Wc).

In one embodiment, W is selected from the group consisting of:

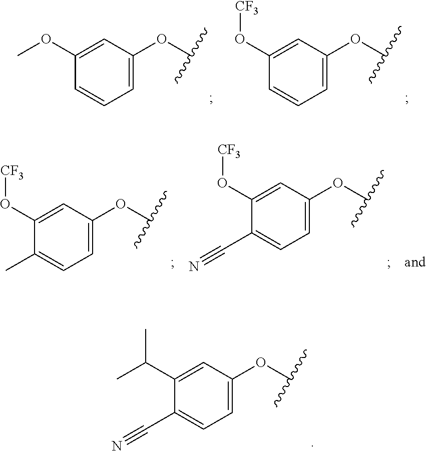

In one embodiment, W is

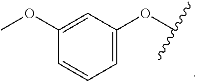 [SMILES: *Oc1cccc(OC)c1]

In one embodiment, W is

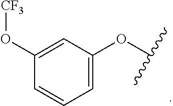 [SMILES: *Oc1cccc(OC(F)(F)F)c1]

In one embodiment, W is

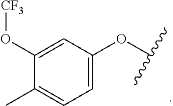 [SMILES: *Oc1ccc(C)c(OC(F)(F)F)c1]

In one embodiment, W is

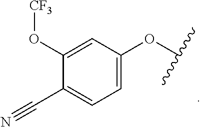 [SMILES: *Oc1ccc(C#N)c(OC(F)(F)F)c1]

In one embodiment, W is

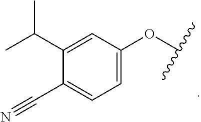 [SMILES: *Oc1ccc(C#N)c(C(C)C)c1]

In one embodiment, the compound of formula (I) is selected from the group consisting of:
3-[2-amino-6-(7-methylspiro[2H-benzofuran-3,1′-cyclopropane]-4-yl)oxy-3-pyridyl]-5,5-dimethyl-imidazolidine-2,4-dione;
(5R)-3-[2-amino-6-(7-methylspiro[2H-benzofuran-3,1′-cyclopropane]-4-yl)oxy-3-pyridyl]-5-ethyl-imidazolidine-2,4-dione;
3-(2-amino-6-spiro[2H-benzofuran-3,1′-cyclopropane]-4-yloxy-3-pyridyl)-5,5-dimethyl-imidazolidine-2,4-dione;
(5R)-3-(2-amino-6-spiro[2H-benzofuran-3,1′-cyclopropane]-4-yloxy-3-pyridyl)-5-ethyl-imidazolidine-2,4-dione;
3-[2-amino-6-[(3,3-dimethyl-2H-benzofuran-4-yl)oxy]-3-pyridyl]-5,5-dimethyl-imidazolidine-2,4-dione;
3-[2-amino-6-(3-methoxyphenoxy)-3-pyridyl]-5,5-dimethyl-imidazolidine-2,4-dione;
3-[2-amino-6-[4-methyl-3-(trifluoromethoxy)phenoxy]-3-pyridyl]-5,5-dimethyl-imidazolidine-2,4-dione;
(5R)-3-[2-amino-6-[4-methyl-3-(trifluoromethoxy)phenoxy]-3-pyridyl]-5-ethyl-imidazolidine-2,4-dione;
3-[2-amino-6-[3-(trifluoromethoxy)phenoxy]-3-pyridyl]-5,5-dimethyl-imidazolidine-2,4-dione;
4-[[6-amino-5-(4,4-dimethyl-2,5-dioxo-imidazolidin-1-yl)-2-pyridyl]oxy]-2-(trifluoromethoxy)benzonitrile;
4-[[6-amino-5-[(4R)-4-ethyl-2,5-dioxo-imidazolidin-1-yl]-2-pyridyl]oxy]-2-(trifluoromethoxy)benzonitrile;
4-[[6-amino-5-(4,4-dimethyl-2,5-dioxo-imidazolidin-1-yl)-2-pyridyl]oxy]-2-isopropyl-benzonitrile; 4-[[6-amino-5-[(4R)-4-ethyl-2,5-dioxo-imidazolidin-1-yl]-2-pyridyl]oxy]-2-isopropyl-benzonitrile; and
3-[2-amino-6-[(3,3,7-trimethyl-2H-benzofuran-4-yl)oxy]-3-pyridyl]-5,5-dimethyl-imidazolidine-2,4-dione.

In one embodiment, there is provided a compound of formula (I) which is 3-[2-amino-6-(7-methylspiro[2H-benzofuran-3,1′-cyclopropane]-4-yl)oxy-3-pyridyl]-5,5-dimethyl-imidazolidine-2,4-dione.

In one embodiment, there is provided a compound of formula (I) which is 3-[2-amino-6-[3-(trifluoromethoxy)phenoxy]-3-pyridyl]-5,5-dimethyl-imidazolidine-2,4-dione.

Such compounds may be provided in the form of a pharmaceutically acceptable salt and/or solvate thereof and/or derivative thereof, such as a pharmaceutically acceptable salt and/or solvate thereof, in particular a pharmaceutically acceptable salt. Optionally, such compounds are not provided in the form of a salt.

Such compounds may also be provided in the form of a salt and/or solvate thereof and/or derivative thereof. Alternatively, such compounds may be in the form of a salt and solvate, in particular a pharmaceutically acceptable salt and solvate.

The term ‘halo’ or ‘halogen’ as used herein, refers to a fluorine, chlorine, bromine or iodine atom. Particular examples of halo are fluorine and chlorine, especially fluorine.

When the compound contains a C₁₋₄alkyl group, whether alone or forming part of a larger group, e.g. C₁₋₄alkoxy, the alkyl group may be straight chain, branched, cyclic, or a combination thereof. Examples of C₁₋₄alkyl are methyl, ethyl, n-propyl, isopropyl, cyclopropyl, n-butyl, isobutyl, sec-butyl, tert-butyl, and cyclobutyl. Reference to “propyl” includes n-propyl, isopropyl and cyclopropyl, and reference to “butyl” includes n-butyl, isobutyl, sec-butyl, tert-butyl and cyclobutyl. A particular group of exemplary C₁₋₄alkyl groups are methyl, ethyl, n-propyl, isopropyl, cyclopropyl, n-butyl, isobutyl, sec-butyl and tert-butyl. Examples of C₁₋₄alkoxy include methoxy, ethoxy, propoxy (which includes n-propoxy, isopropoxy and cyclopropoxy) and butoxy.

The term “C₁₋₄alkoxy” also extends to embodiments in which the oxygen atom is located within the alkyl chain, for example —CH₂CH₂OCH₃ or —CH₂OCH₃. Thus in one embodiment the alkoxy is linked through carbon to the remainder of the molecule. In one embodiment the alkoxy is linked through oxygen to the remainder of the molecule, for example —OC₁₋₄alkyl.

The term ‘haloC₁₋₄alkyl’ as used herein, includes straight chain, branched chain or cyclic alkyl groups containing 1 to 4 carbon atoms substituted by one or more halo atoms, for example fluoromethyl, difluoromethyl and trifluoromethyl. A particular group of exemplary haloC₁₋₄ alkyl include methyl and ethyl groups substituted with one to three halo atoms, in particular one to three fluoro atoms, such as trifluoromethyl or 2,2,2-trifluoroethyl.

The term ‘haloC₁₋₄alkoxy’ as used herein, includes straight chain, branched chain or cyclic alkoxy groups containing 1 to 4 carbon atoms substituted by one or more halo atoms, for example fluoromethoxy, difluoromethoxy and trifluoromethoxy. A particular group of exemplary haloC₁₋₄ alkyl include methoxy and ethoxy groups substituted with one to three halo atoms, in particular one to three fluoro atoms.

The term ‘C₃₋₅ spiro carbocyclyl’ as used herein means a cyclic ring system containing 3 to 5 carbon atoms, for example, a cyclopropyl, cyclobutyl or cyclopentyl group, wherein the cyclic ring system is attached to a secondary carbon via a spirocentre such that the secondary carbon is one of the 3 to 5 carbon atoms in the cyclic ring as follows:

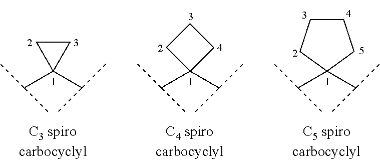

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge et al. (1977). Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Typically, the phrase “pharmaceutically acceptable” is used to refer to those compounds, materials, compositions, dosage forms and the like which are, within the scope of sound medical judgment, suitable for use in contact with the tissues of human beings and animals, for example human beings, without excessive toxicity, irritation, allergic response, or other problem or complication, commensurate with a reasonable benefit/risk ratio.

Certain of the compounds of formula (I) may form acid addition salts with one or more equivalents of the acid. The present invention includes within its scope all possible stoichiometric and non-stoichiometric forms.

In some suitable compounds, the compound of formula (I) is the free base form. Alternatively, there is provided a compound of formula (I) in the form of a free acid. When the compound contains a basic group as well as the free acid it may be Zwitterionic.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

It will be understood that the invention includes pharmaceutically acceptable derivatives of compounds of formula (I) and that these are included within the scope of the invention.

As used herein “pharmaceutically acceptable derivative” includes any pharmaceutically acceptable prodrug such as an ester or salt of such ester of a compound of formula (I) which, upon administration to the recipient is capable of providing (directly or indirectly) a compound of formula (I) or an active metabolite or residue thereof.

Suitably, a pharmaceutically acceptable prodrug is formed by functionalising the secondary nitrogen of the hydantoin, or the secondary nitrogen of the triazolone, for example with a group “L” as illustrated below:

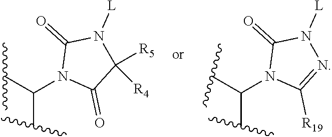

In one embodiment of the invention, a compound of formula (I) is functionalised via the secondary nitrogen of the hydantoin or via the secondary nitrogen of the triazolone with a group L as illustrated directly above, wherein L is selected from:
a) —PO(OH)O⁻·M⁺, wherein M⁺ is a pharmaceutically acceptable monovalent counterion,
b) —PO(O⁻)₂·2M⁺,
c) —PO(O⁻)₂·D²⁺, wherein D²⁺ is a pharmaceutically acceptable divalent counterion,
d) —CH(R^{X})—PO(OH)O⁻·M⁺, wherein R^{X} is hydrogen or C₁₋₃ alkyl,
e) —CH(R^{X})—PO(O⁻)₂·2M⁺,
f) —CH(R^{X})—PO(O⁻)₂·D²⁺,
g) —SO₃⁻·M⁺,
h) —CH(R^{X})—SO₃⁻·M⁺, and
i) —CO—CH₂CH₂—CO₂·M⁺.

It is to be understood that the present invention encompasses all isomers of formula (I) and their derivatives (e.g. pharmaceutically acceptable derivatives), including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

Where compounds exist as two or more optical isomeric forms, one enantiomer in a pair of enantiomers may exhibit advantages over the other enantiomer, for example, in terms of biological activity. Thus, in certain circumstances, it may be desirable to use only one of a pair of enantiomers, or only one of a plurality of diastereoisomers. Accordingly, the invention provides compositions containing a compound of the invention having one or more chiral centres, wherein at least 55% (e.g. at least 60%, 65%, 70%, 75%, 80%, 85%, 90% or 95%) of the compound of the invention is present as a single optical isomer (e.g. enantiomer or diastereoisomer). Suitably, 99% or more (e.g. substantially all) of the total amount of the compound of the invention may be present as a single optical isomer (e.g. enantiomer or diastereoisomer).

The subject invention also includes isotopically-labelled compounds which are identical to those recited in formula (I) but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”). Examples of isotopes that can be incorporated into compounds of the invention include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I (e.g ³H, ¹¹C, ¹⁴C, ¹⁸F, ¹²³I or ¹²⁵I), which may be naturally occurring or non-naturally occurring isotopes.

Compounds of the present invention and pharmaceutically acceptable salts of said compounds that contain the aforementioned isotopes and/or other isotopes of other atoms are within the scope of the present invention. Isotopically labelled compounds of the present invention, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

The invention also includes all isotopic forms of the compounds of the invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

In some suitable compounds of the invention, the compounds are provided in a natural isotopic form.

In some suitable compounds of the invention, the compounds are provided in an unnatural variant isotopic. Suitably, the atoms of the compounds of the invention are in an isotopic form which is not radioactive. Suitably the unnatural variant isotopic form is a pharmaceutically acceptable form.

Suitably, a compound of the invention is provided whereby a single atom of the compound exists in an unnatural variant isotopic form. Alternatively, a compound of the invention is provided whereby two or more atoms exist in an unnatural variant isotopic form.

Unnatural isotopic variant forms can generally be prepared by conventional techniques known to those skilled in the art or by processes analogous to those described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

Since the compounds of formula (I) and/or a pharmaceutically acceptable salt and/or solvate and/or derivative thereof are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

Suitably the compounds of the invention are provided in the form of the free base.

In general, the compounds of formula (I) may be made according to the organic synthesis techniques known to those skilled in this field, as well as by the representative methods set forth below, those in the Examples, and modifications thereof.

Patent applications WO2011/069951, WO2012/076877, WO2012/168710, WO2013/175215, WO2013/083994, WO2017/098254, WO2017/103604, WO2018/020263, WO2018/109484, WO2020/079422, WO2021/156584 and WO2023/017263 provide methods for the synthesis of intermediates which may be of use in the production of compounds of the present invention.

### General Synthesis Schemes

The following schemes detail synthetic routes to compounds of the invention and intermediates in the synthesis of such compounds. In the following schemes reactive groups can be protected with protecting groups and deprotected according to established techniques well known to the skilled person. In the following schemes X and Y are both H.

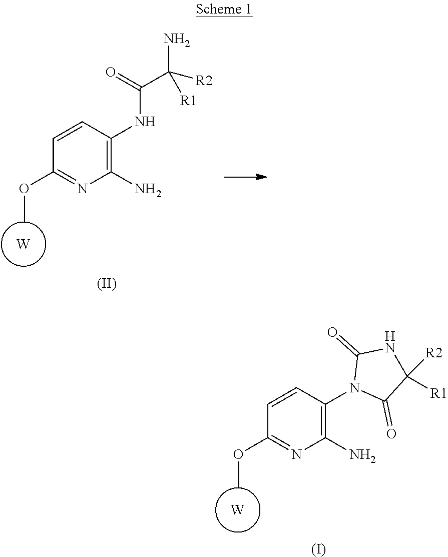

Compounds of formula (I) can be prepared by cyclization of compounds of formula (II) in a suitable solvent e.g. dichloromethane with a carbonylating agent e.g. triphosgene preferentially prediluted in the same solvent and added in a second time at 0° C. in presence of a suitable base e.g. triethylamine.

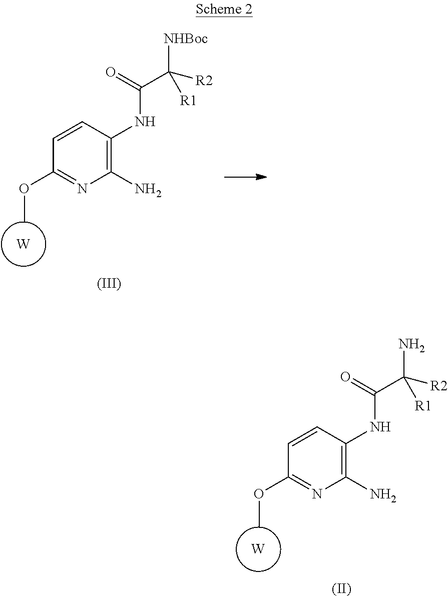

Compounds of formula (II) can be prepared by the cleavage of the tert-butyloxycarbonyl protecting group of compounds of formula (III). In this reaction the Boc-derivative of formula (III) is reacted at 0° C. in the presence of strong acids such as trifluoroacetic acid in a suitable solvent such as dichloromethane.

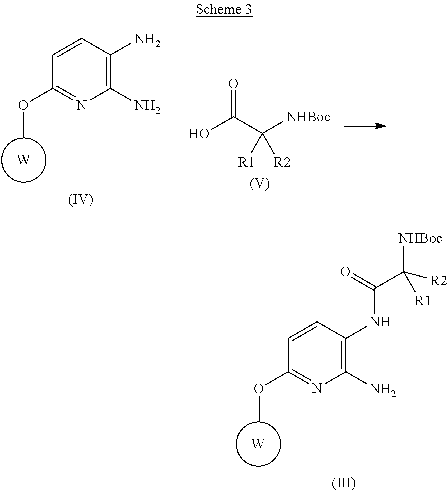

Compound of formula (III) can be prepared by an amide coupling reaction. In the reaction the diamine derivate of formula (IV) and an amino acid Boc protected of formula (V) are reacted in the presence of a coupling agent such as HATU in the presence of a suitable base such as DIPEA in a suitable solvent such as N,N-dimethylformamide, stirring at room temperature or with conventional heating.

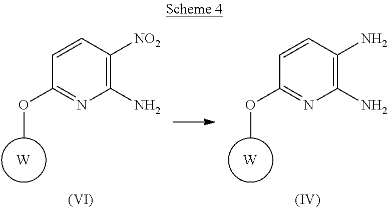

Dianiline derivatives of formula (IV) can be prepared by the reduction of the nitro-amino-pyridine derivatives of formula (VI) employing suitable reaction conditions such as iron powder and ammonium chloride in a suitable solvent such as a mixture of ethanol and water or a mixture of ethanol, water and tetrahydrofuran, with conventional heating.

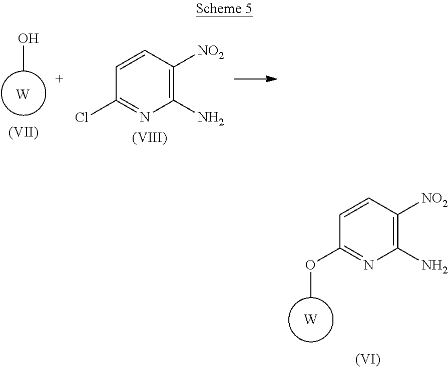

Compounds of formula (VI) can be prepared by nucleophilic aromatic substitution. In this reaction a phenol derivative of formula (VII) and 2-amino-6-chloro-3-nitropyridine of formula (VIII) are reacted in the presence of a suitable base such as potassium carbonate in a suitable solvent such as N,N′-dimethylacetamide, with conventional heating.

### Processes of the Invention

According to further aspects of the present invention are provided processes for the preparation of compounds of formula (I) and derivatives thereof, as well as processes for preparing intermediates in the synthesis of compounds of formula (I). The processes of the invention are described above an include any individual step of a multi-step scheme.

According to further aspects of the present invention are also provided processes for the preparation of compounds of formula (I) and/or salts and/or solvates and/or derivatives thereof, as well as processes for preparing intermediates in the synthesis of compounds of formula (I) and/or salts and/or solvates and/or derivatives thereof.

### Intermediates of the Invention

The present invention also relates to novel intermediates in the synthesis of compounds of formula (I), such as compounds of formulae (II), (Ill), (IV), (V), (VI) and (VII). Salts, such as pharmaceutically acceptable salts, of such intermediates are also provided by the present invention.

Intermediates of the invention therefore include:
a compound of formula (II):

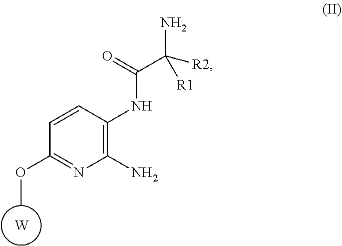

wherein W, R₁ and R₂ are as defined for the compound of formula (I); or
a compound of formula (III):

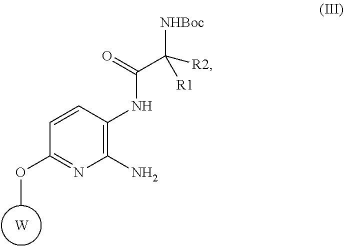

wherein W, R₁ and R₂ are as defined for the compound of formula (I); or
wherein R₁ and R₂ are as defined for the compound of formula (I); or
a compound of formula (IV):

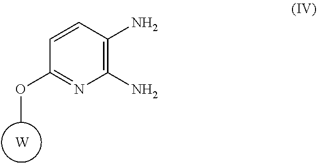

a compound of formula (V)

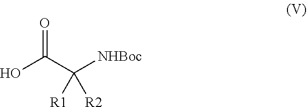

wherein W is as defined for the compound of formula (I); or
a compound of formula (VI):

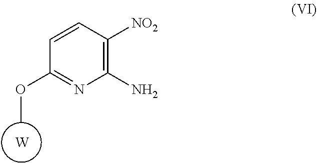

wherein W is as defined for the compound of formula (I); or
a compound of formula (VII):

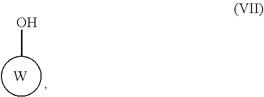

wherein W is as defined for the compound of formula (I).
Kv3.1, Kv3.2, Kv3.3 and/or Kv3.4 Modulation

Compounds of formula (I) of the present invention are modulators of Kv3.1. Compounds of formula (I) may also be modulators of Kv3.2, Kv3.3 and/or Kv3.4. Compounds of the invention may be tested in the assay of Biological Example 1 to determine their modulatory properties for Kv3.1 and/or Kv3.2 and/or Kv3.3 and/or Kv3.4 channels.

A ‘modulator’ as used herein refers to a compound which is capable of producing at least 10% potentiation, and suitably at least 20% potentiation of whole-cell currents mediated by human Kv3.1 and/or human Kv3.2 and/or human Kv3.3 and/or human Kv3.4 channels recombinantly expressed in mammalian cells.

The term ‘Kv3.1, Kv3.2, Kv3.3 and/or Kv3.4’ shall be taken to mean the same as ‘Kv3.1 and/or Kv3.2 and/or Kv3.3 and/or Kv3.4’ and may also be referred to as ‘Kv3.1/Kv3.2/Kv3.3/Kv3.4’.

In one embodiment the modulator is capable of producing at least 10% potentiation and suitably at least 20% potentiation of whole-cell currents mediated by human Kv3.1 channels recombinantly expressed in mammalian cells. Suitably the pEC₅₀ of the modulator is in the range of 4-8 (such as 5-7.5).

In one embodiment the modulator is capable of producing at least 10% potentiation and suitably at least 20% potentiation of whole-cell currents mediated by human Kv3.2 channels recombinantly expressed in mammalian cells. Suitably the pEC₅₀ of the modulator is in the range of 4-8 (such as 5-7.5).

In one embodiment the modulator is capable of producing at least 10% potentiation and suitably at least 20% potentiation of whole-cell currents mediated by human Kv3.3 channels recombinantly expressed in mammalian cells. Suitably the pEC₅₀ of the modulator is in the range of 4-8 (such as 5-7.5).

In one embodiment the modulator is capable of producing at least 10% potentiation and suitably at least 20% potentiation of whole-cell currents mediated by human Kv3.4 channels recombinantly expressed in mammalian cells. Suitably the pEC₅₀ of the modulator is in the range of 4-8 (such as 5-7.5).

In another embodiment the modulator is capable of producing at least 10% potentiation and suitably at least 20% potentiation of whole-cell currents mediated by human Kv3.1 and Kv3.2 channels recombinantly expressed in mammalian cells.

In another embodiment the modulator is capable of producing at least 10% potentiation and suitably at least 20% potentiation of whole-cell currents mediated by human Kv3.1 and Kv3.3 channels recombinantly expressed in mammalian cells.

In another embodiment the modulator is capable of producing at least 10% potentiation and suitably at least 20% potentiation of whole-cell currents mediated by human Kv3.2 and Kv3.3 channels recombinantly expressed in mammalian cells.

In a further embodiment the modulator is capable of producing at least 10% potentiation and suitably at least 20% potentiation of whole-cell currents mediated by human Kv3.1, Kv3.2 and Kv3.3 channels recombinantly expressed in mammalian cells.

In one embodiment of the invention the compounds of formula (I) or their pharmaceutically acceptable salts and/or solvates and/or derivatives thereof are selective for modulation of Kv3.1 channels over modulation of Kv3.2 channels. By selective, is meant that compounds demonstrate, for example, at least a 2 fold, 5 fold or 10 fold activity for Kv3.1 channels than for Kv3.2 channels. The activity of a compound is suitably quantified by its potency as indicated by an EC50 value.

In another embodiment of the invention, the compounds of formula (I) or their pharmaceutically acceptable salts and/or solvates and/or derivatives thereof are selective for modulation of Kv3.2 channels over modulation of Kv3.1 channels. Once again, by selective is meant that compounds demonstrate, for example at least a 2 fold, 5 fold or 10 fold activity for Kv3.2 channels than for Kv3.1 channels.

In a particular embodiment of the invention the compounds of formula (I) or their pharmaceutically acceptable salts and/or solvates and/or derivatives thereof demonstrate comparable activity between modulation of Kv3.1 and Kv3.2 channels, for example the activity for one channel is less than 2 fold that for the other channel, such as less than 1.5 fold or less than 1.2 fold.

In certain disorders it may be of benefit to utilise a modulator of Kv3.3 or Kv3.1, or Kv3.3 and Kv3.1 which demonstrates a particular selectivity profile between the two channels. For example a compound may be selective for modulation of Kv3.3 channels over modulation of Kv3.1 channels demonstrating, for example, at least a 2 fold, 5 fold or 10 fold activity for Kv3.3 channels than for Kv3.1 channels.

In another embodiment of the invention, the compounds of formula (I) or their pharmaceutically acceptable salts and/or solvates and/or derivatives thereof are selective for modulation of Kv3.1 channels over modulation of Kv3.3 channels. Once again, by selective is meant that compounds demonstrate, for example at least a 2 fold, 5 fold or 10 fold activity for Kv3.1 channels than for Kv3.3 channels.

In a particular embodiment of the invention, a compound may demonstrate comparable activity between modulation of Kv3.3 and Kv3.1 channels, for example the activity for each channel is less than 2 fold that for the other channel, such as less than 1.5 fold or less than 1.2 fold.

In certain disorders it may be of benefit to utilise a modulator of Kv3.3 or Kv3.2, or Kv3.3 and Kv3.2 which demonstrates a particular selectivity profile between the two channels. A compound may be selective for modulation of Kv3.3 channels over modulation of Kv3.2 channels demonstrating, for example, at least a 2 fold, 5 fold or 10 fold activity for Kv3.3 channels than for Kv3.2 channels.

In another embodiment of the invention, the compounds of formula (I) or their pharmaceutically acceptable salts and/or solvates and/or derivatives thereof are selective for modulation of Kv3.2 channels over modulation of Kv3.3 channels. Once again, by selective is meant that compounds demonstrate, for example at least a 2 fold, 5 fold or 10 fold activity for Kv3.2 channels than for Kv3.3 channels.

In another particular embodiment a compound may demonstrate comparable activity between modulation of Kv3.3 and Kv3.2 channels, for example the activity for each channel is less than 2 fold that for the other channel, such as less than 1.5 fold or less than 1.2 fold.

In a yet further particular embodiment of the invention a compound may demonstrate comparable activity between modulation of Kv3.3, Kv3.2 and Kv3.1 channels, for example the activity for each channel is less than 2 fold that for any other channel, such as less than 1.5 fold or less than 1.2 fold. The activity of a compound is suitably quantified by its potency as indicated by an EC50 value.

### Therapeutic Methods

The invention also provides a compound of formula (I), or a pharmaceutically acceptable salt and/or solvate (e.g. salt) and/or derivative thereof, for use in the treatment or prophylaxis of a disease or disorder where a modulator of Kv3.1, Kv3.2, Kv3.3 and/or Kv3.4 is required, for example those diseases and disorders mentioned herein below.

The invention provides a method of treating or preventing a disease or disorder where a modulator of Kv3.1, Kv3.2, Kv3.3 and/or Kv3.4 is required, for example those diseases and disorders mentioned herein below, which comprises administering to a subject a compound of formula (I) or a pharmaceutically acceptable salt and/or solvate (e.g. salt) and/or derivative thereof.

The invention also provides the use of a compound of formula (I), or a pharmaceutically acceptable salt and/or solvate thereof (e.g. salt) and/or derivative, in the manufacture of a medicament for the treatment or prophylaxis of a disease or disorder where a modulator of Kv3.1, Kv3.2, Kv3.3 and/or Kv3.4 is required, for example those diseases and disorders mentioned herein below.

In one embodiment is provided a compound of formula (I) or a pharmaceutically acceptable salt and/or solvate thereof and/or derivative thereof for use as a medicament.

In another embodiment is provided the use of a compound of formula (I) or a pharmaceutically acceptable salt and/or solvate and/or derivative thereof in the manufacture of a medicament.

The term “treatment” or “treating” as used herein includes the control, mitigation, reduction, or modulation of the disease state or its symptoms.

The term “prophylaxis” is used herein to mean preventing symptoms of a disease or disorder in a subject or preventing recurrence of symptoms of a disease or disorder in an afflicted subject and is not limited to complete prevention of an affliction.

A subject will typically be a subject in need of treatment or prophylaxis according to the invention. A subject is typically a mammal. Suitably the subject is a human.

Diseases or disorders that may be mediated by modulation of Kv3.1 and/or Kv3.2 channels may be selected from the list below. The numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10).

In one embodiment of the invention, the compounds of formula (I) or their pharmaceutically acceptable salts and/or solvates and/or derivatives may be of use for the treatment or prophylaxis of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Ménière's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease.

In one embodiment of the invention, the compounds of formula (I) or their pharmaceutically acceptable salts and/or solvates and/or derivatives may be of use for the treatment or prophylaxis of a disease or disorder selected from the group consisting of hearing disorders including hearing loss and tinnitus, schizophrenia, substance abuse disorders, pain such as neuropathic pain, inflammatory pain and miscellaneous pain, Lewy body dementia and Parkinson's disease.

In one embodiment of the invention, the compounds of formula (I) or their pharmaceutically acceptable salts and/or solvates and/or derivatives may be of use for the treatment or prophylaxis of a disease or disorder selected from the group consisting of Fragile-X, Rett's Disorder and Alzheimer's disease.

The invention provides a method for the prophylaxis or treatment of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Meniere's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease, which comprises administering to a subject a compound of formula (I) or a pharmaceutically acceptable salt and/or solvate (e.g. salt) and/or derivative thereof.

The invention also provides the use of a compound of formula (I), or a pharmaceutically acceptable salt and/or solvate thereof (e.g. salt) and/or derivative thereof, in the manufacture of a medicament for the treatment or prophylaxis of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Ménière's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease.

In a particular embodiment of the invention, there is provided a compound of formula (I) or their pharmaceutically acceptable salts and/or solvates (e.g. salts) and/or derivatives thereof for use in the treatment of prophylaxis of hearing disorders. Hearing disorders include auditory neuropathy, auditory processing disorder, hearing loss, which includes sudden hearing loss, noise induced hearing loss, substance-induced hearing loss, and hearing loss in adults over 60, over 65, over 70 or over 75 years of age (presbycusis), and tinnitus.

The compounds of formula (I) or their pharmaceutically acceptable salts and/or solvates (e.g. salts) and/or derivatives thereof may be of use for the treatment or prophylaxis of Ménière's disease, disorders of balance, and disorders of the inner ear.

In a particular embodiment of the invention, there is provided a compound of formula (I) or their pharmaceutically acceptable salts and/or solvates (e.g. salts) and/or derivatives thereof for use in the treatment or prophylaxis of schizophrenia. Schizophrenia includes the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) or their pharmaceutically acceptable salts and/or solvates (e.g. salts) and/or derivatives thereof may be of use for the treatment or prophylaxis of depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90); Seasonal affective disorder.

The compounds of formula (I) or their pharmaceutically acceptable salts and/or solvates (e.g. salts) and/or derivatives thereof may be of use for the treatment or prophylaxis of Epilepsy, (including, but not limited to, localization-related epilepsies, generalized epilepsies, epilepsies with both generalized and local seizures, and the like), seizures associated with Lennox-Gastaut syndrome, seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, fever, infection, and the like), essential tremor, restless limb syndrome, partial and generalised seizures (including tonic, clonic, tonic-clonic, atonic, myoclonic, absence seizures), secondarily generalized seizures, temporal lobe epilepsy, absence epilepsies (including childhood, juvenile, myoclonic, photo- and pattern-induced), severe epileptic encephalopathies (including hypoxia-related and Rasmussen's syndrome), febrile convulsions, epilepsy partialis continua, progressive myoclonus epilepsies (including Unverricht-Lundborg disease and Lafora's disease), post-traumatic seizures/epilepsy including those related to head injury, simple reflex epilepsies (including photosensive, somatosensory and proprioceptive, audiogenic and vestibular), metabolic disorders commonly associated with epilepsy such as pyridoxine-dependent epilepsy, Menkes' kinky hair disease, Krabbe's disease, epilepsy due to alcohol and drug abuse (e.g. cocaine), cortical malformations associated with epilepsy (e.g. double cortex syndrome or subcortical band heterotopia), chromosomal anomalies associated with seizures or epilepsy such as Partial monosomy (15Q)/Angelman syndrome).

The compounds of formula (I) or their pharmaceutically acceptable salts and/or solvates (e.g. salts) and/or derivatives thereof may be of use for the treatment or prophylaxis of substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) or their pharmaceutically acceptable salts and/or solvates (e.g. salts) and/or derivatives thereof may be of use for the treatment or prophylaxis of anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

The compounds of formula (I) or their pharmaceutically acceptable salts and/or solvates (e.g. salts) and/or derivatives thereof may be of use for the treatment or prophylaxis of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome.

The compounds of formula (I) or their pharmaceutically acceptable salts and/or solvates (e.g. salts) and/or derivatives thereof may be of use for the treatment or prophylaxis of hyperacusis and disturbances of loudness perception, including Fragile-X syndrome and autism.

The compounds of formula (I) or their pharmaceutically acceptable salts and/or solvates (e.g. salts) and/or derivatives thereof may be of use for the treatment or prophylaxis of Impulse control disorder including: Intermittent Explosive Disorder (312.34), Kleptomania (312.32), Pathological Gambling (312.31), Pyromania (312.33), Trichotillomania (312.39), Impulse-Control Disorders Not Otherwise Specified (312.3), Binge Eating, Compulsive Buying, Compulsive Sexual Behaviour and Compulsive Hoarding.

The compounds of formula (I) or their pharmaceutically acceptable salts and/or solvates (e.g. salts) and/or derivatives thereof may be of use for the treatment or prophylaxis of Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of formula (I) or their pharmaceutically acceptable salts and/or solvates (e.g. salts) and/or derivatives thereof may be of use for the treatment or prophylaxis of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of formula (I) or their pharmaceutically acceptable salts and/or solvates (e.g. salts) and/or derivatives thereof may be of use for the treatment or prophylaxis of Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) or their pharmaceutically acceptable salts and/or solvates (e.g. salts) and/or derivatives thereof may be of use for the treatment or prophylaxis of Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).

The compounds of formula (I) or their pharmaceutically acceptable salts and/or solvates (e.g. salts) and/or derivatives thereof may be of use for the treatment or prophylaxis of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50).

The compounds of formula (I) or their pharmaceutically acceptable salts and/or solvates (e.g. salts) and/or derivatives thereof may be of use for the enhancement of cognition including the treatment of cognition impairment in other diseases such as schizophrenia, bipolar disorder, depression, other psychiatric disorders and psychotic conditions associated with cognitive impairment, e.g. Alzheimer's disease. Alternatively, the compounds of formula (I) or their pharmaceutically acceptable salts and/or solvates thereof may be of use for the prophylaxis of cognition impairment, such as may be associated with in diseases such as schizophrenia, bipolar disorder, depression, other psychiatric disorders and psychotic conditions associated with cognitive impairment, e.g. Alzheimer's disease.

The compounds of formula (I) or their pharmaceutically acceptable salts and/or solvates (e.g. salts) and/or derivatives thereof may be of use for the treatment or prophylaxis of ataxia including ataxia, in particular spinocerebellar ataxia, especially ataxia associated with R420H, R423H or F448L mutations.

The compounds of formula (I) or their pharmaceutically acceptable salts and/or solvates (e.g. salts) and/or derivatives thereof may be of use for the treatment or prophylaxis of pain including nociceptive, neuropathic, inflammatory or miscellaneous pain.

Nociceptive pain represents the normal response to noxious insult or injury of tissues such as skin, muscles, visceral organs, joints, tendons, or bones. Examples of nociceptive pain which form part of the invention include somatic pain: musculoskeletal (joint pain, myofascial pain) or cutaneous, which is often well localized; or visceral pain: hollow organs or smooth muscle.

Neuropathic pain is pain initiated or caused by a primary lesion or disease in the somatosensory nervous system. Sensory abnormalities range from deficits perceived as paraesthesia (numbness) to hypersensitivity (hyperalgesia or allodynia), and dysaesthesia (tingling and other sensations). Examples of neuropathic pain which form part of the invention include, but are not limited to, diabetic neuropathy, post-herpetic neuralgia, spinal cord injury pain, phantom limb (post-amputation) pain, and post-stroke central pain. Other causes of neuropathic pain include trauma, chemotherapy and heavy metal exposure.

Inflammatory pain occurs as a result of activation and sensitization of the nociceptive pain pathway by a variety of mediators released at a site of tissue inflammation. Mediators that have been implicated as key players in inflammatory pain are pro-inflammatory cytokines such IL-1-alpha, IL-1-beta, IL-6 and TNF-alpha, chemokines, reactive oxygen species, vasoactive amines, lipids, ATP, acid, and other factors released by infiltrating leukocytes, vascular endothelial cells, or tissue resident mast cells. Examples causes of inflammatory pain which form part of the invention include appendicitis, rheumatoid arthritis, inflammatory bowel disease, and herpes zoster.

Miscellaneous pain refers to pain conditions or disorders which are not easily classifiable. The current understanding of their underlying mechanisms is still rudimentary though specific therapies for those disorders are well known; they include cancer pain, migraine and other primary headaches and wide-spread pain of the fibromyalgia type.

Suitably, specific pain indications that may be mediated by a modulator of Kv3.1 and/or Kv3.2 and/or Kv3.3 and/or Kv3.4 channels are neuropathic pain and/or inflammatory pain.

Pain is a subjective condition and in a clinical setting tends to be measured by a patient's self-assessment. Therefore it can be difficult to measure and quantify pain threshold. For chronic pain, typically a subjective 11-point rating scale is used where 0 is no pain and 10 is the worst pain imaginable. Subjects generally record their worst pain over a given period, usually a day. A minimum mean baseline score is also recorded and response to the medication is measured relative to the baseline, for example, a reduction of at least 10%, 20%, 30%, 40% or 50% in pain from the baseline score may be observed.

Since individual responses to medicaments may vary, not all individuals may experience a reduction in pain from the baseline score. Consequently, suitably a reduction is observed in at least at least 10%, 20%, 30%, 40%, 50%, 60%, 70%, 80%, 90% or all individuals tested.

Therefore, in one embodiment of the invention, a reduction of at least 10%, 20%, 30%, 40% or 50% in pain from the baseline score is observed upon administration of a compound of formula (I) or a pharmaceutically acceptable salt, solvate and/or derivative thereof to a subject.

Administration can occur before an anticipated onset of pain or after the onset of pain. In cases where it is anticipated that development of a disease or disorder may lead to an increase in pain experienced by the subject, a compound of formula (I) or a pharmaceutically acceptable salt, solvate and/or derivative thereof can be administered. In cases where a subject is already experiencing pain a compound of formula (I) or a pharmaceutically acceptable salt, solvate and/or derivative thereof may be administered to a subject.

Treatment of the subject may continue for as long as treatment is required, for example, 1 day, 1 week, 2 weeks, 3 weeks, 1 month, 6 months, 1 year, more than 1 year more than 2 years, more than 5 years or more than 10 years. Therefore in one embodiment of the invention a compound of formula (I) or a pharmaceutically acceptable salt, solvate and/or derivative thereof, is administered to a subject for 1 day to 1 month, 1 week to 3 months, 1 month to 6 months, 3 months to 1 year or more than 1 year.

Reduction in pain in a subject can be measured by assessing the response to an external stimuli such as mechanical or thermal (e.g. cold) stimuli (such as described in the Experimental section). The reduction can either be considered as a percentage reversal (calculated by measuring the pre- and post-dose thresholds of the affected pain site with a non-affected pain site, such as described in more detail under Data Analysis in the Experimental Section) or by measuring withdrawal thresholds of the affected pain site. Preferably, the percentage reversal calculation is used.

Therefore, in one embodiment of the invention, the sensitivity to pain (such as neuropathic pain or inflammatory pain) is reversed by more than 20%, more than 30%, more than 40%, more than 50%, more than 60%, more than 70%, more than 80% or more than 90%, upon administration of a compound of formula (I) or a pharmaceutically acceptable salt, solvate and/or derivative thereof. Suitably, the sensitivity to pain is reversed by more than 80% or more than 90%.

Subjects may experience secondary benefits, such as one or more of improved function, mood, sleep, quality of life, reduced time off work.

In a particular embodiment, the compounds of formula (I) or their pharmaceutically acceptable salts and/or solvates (e.g. salts) and/or derivatives thereof may be of use for the treatment or prophylaxis of neuropathic pain.

In a particular embodiment, the compounds of formula (I) or their pharmaceutically acceptable salts and/or solvates (e.g. salts) and/or derivatives thereof may be of use for the treatment or prophylaxis of inflammatory pain.

In a particular embodiment, the compounds of formula (I) or their pharmaceutically acceptable salts and/or solvates (e.g. salts) and/or derivatives thereof may be of use for the treatment or prophylaxis of miscellaneous pain.

In some embodiments the pain is chronic pain.

In one embodiment is provided a compound of formula (I) for use in the prophylaxis of acute noise-induced hearing loss.

In one embodiment is provided a method for the prophylaxis of acute noise-induced hearing loss, comprising administering to a subject a compound of formula (I).

In one embodiment is provided the use of a compound of formula (I) in the manufacture of a medicament for the prophylaxis of acute noise-induced hearing loss.

Acute noise-induced hearing loss may be caused by events such as exposure to loud noise or a blast. In these cases, where it is anticipated that a future event may result in acute noise-induced hearing loss, the compound of formula (I) or a pharmaceutically acceptable salt, solvate and/or derivative thereof may be administered before the event in order to prevent or reduce acute noise-induced hearing loss. The administration of compound (1) or a pharmaceutically acceptable salt, solvate and/or derivative thereof may prevent any acute noise-induced hearing loss, or may reduce the severity of the acute noise-induced hearing loss or may mitigate other symptoms arising from acute noise-induced hearing loss, such as tinnitus.

“Acute hearing loss” is defined as hearing loss which occurs rapidly over a period of hours or days. For example, hearing loss may occur over a period of minutes, hours or days (for example over a period of up to 1 day, such as up to 2 days, 3 days, 4 days, 5 days, 6 days or 7 days). Acute hearing loss will typically be caused by exposure to loud sound or blast. Hearing loss caused by exposure to loud sound or blast is referred to herein as “noise-induced induced hearing loss”. “Acute noise induced hearing loss” is therefore hearing loss which occurs rapidly over a period of hours or days caused by exposure to loud sound or blast.

Important symptoms of acute hearing loss include:
1. a shift in the auditory threshold, i.e. an increase in the minimum sound level of a pure tone that can be heard with no other sound present;
2. tinnitus; and
3. degradation in central auditory processing, for example impaired auditory temporal processing and/or speech understanding.

A “loud” noise or blast may be at least 90 dB, for example, at least 100 dB, at least 110 dB, at least 120 dB or at least 130 dB.

In one embodiment, administration of the compound of formula (I) or a pharmaceutically acceptable salt, solvate and/or derivative thereof is initiated before an event which is anticipated to cause noise-induced acute hearing loss. For example, administration of the compound of formula (I) or a pharmaceutically acceptable salt, solvate and/or derivative thereof may be initiated up to 2 weeks in advance, such as up to 1 week, 6 days, 5 days, 4 days, 3 days, 2 days, 24 h, 12 h, 6 h, 5 h, 4 h, 3 h, 2 h, 1 h, 30 minutes or up to 15 minutes in advance of an event which is anticipated to cause noise-induced acute hearing loss. The compound of formula (I) or a pharmaceutically acceptable salt, solvate and/or derivative thereof may be administered on multiple occasions before event which is anticipated to cause noise-induced acute hearing loss.

In one embodiment, a compound of formula (I) or a pharmaceutically acceptable salt, solvate and/or derivative thereof is administered in advance of potential exposure to a noise or blast which is anticipated to cause acute noise-induced hearing loss, for preventing or reducing the development of permanent tinnitus; for preventing or reducing the development of a permanent shift in auditory thresholds; or for preventing or reducing the development of permanently degraded central auditory processing, including for example auditory temporal processing and/or speech understanding.

It will be appreciated that administration in advance may be in circumstances where the subject is considered to be at risk of exposure to a noise or blast which is anticipated to cause acute noise-induced hearing loss and is not limited to those circumstances where such exposure ultimately occurs.

In one embodiment, administration of the compound of formula (I) or a pharmaceutically acceptable salt, solvate and/or derivative thereof is initiated during an event which is anticipated to cause noise-induced acute hearing loss. The compound of formula (I) or a pharmaceutically acceptable salt, solvate and/or derivative thereof may be administered on multiple occasions during an event which is anticipated to cause noise-induced acute hearing loss.

In one embodiment, a compound of formula (I) or a pharmaceutically acceptable salt, solvate and/or derivative thereof is initially administered during a noise or blast which is anticipated to cause acute noise-induced hearing loss, for preventing or reducing the development of permanent tinnitus; for preventing or reducing the development of a permanent shift in the auditory threshold; or for preventing or reducing the development of permanently degraded central auditory processing, including for example auditory temporal processing and/or speech understanding.

In one embodiment, administration of the compound of formula (I) or a pharmaceutically acceptable salt, solvate and/or derivative thereof is initiated after an event which is anticipated to cause acute noise-induced hearing loss.

Thus, in one embodiment, a compound of formula (I) or a pharmaceutically acceptable salt, solvate and/or derivative thereof is initially administered after a noise or blast which is anticipated to cause acute noise-induced hearing loss, for preventing or reducing the development of permanent tinnitus; for preventing or reducing the development of a permanent shift in the auditory threshold; or for preventing or reducing the development of permanently degraded central auditory processing, including for example auditory temporal processing and/or speech understanding.

When the compound of formula (I) is administered after an event which is anticipated to cause acute noise-induced hearing loss, such administration is normally undertaken during the “acute phase” i.e. before the hearing loss has become established.

In one embodiment, administration of the compound of formula (I) or a pharmaceutically acceptable salt, solvate and/or derivative thereof may be initiated up to 2 months after an event which is anticipated to cause noise-induced acute hearing loss, such as up to 1 month, 2 weeks, 1 week, 6 days, 5 days, 4 days, 3 days, 2 days, 24 h, 12 h, 6 h, 5 h, 4 h, 3 h, 2 h, 1 h, 30 minutes or up to 15 minutes after an event which is anticipated to cause acute noise-induced hearing loss. The compound of formula (I) or a pharmaceutically acceptable salt, solvate and/or derivative thereof may be administered on multiple occasions after an event which is anticipated to cause noise-induced acute hearing loss.

The compound of formula (I) or a pharmaceutically acceptable salt, solvate and/or derivative thereof may be administered over a period of up to 7 days (for example, up to 1 day, up to 2 days, up to 3 days, up to 4 days, up to 5 days, up to 6 days or up to 7 days), for 1-2 weeks (for example, 7-8 days, 7-9 days, 7-10 days, 7-11 days, 7-12 days, 7-13 days or 7-14 days), for 2-4 weeks (for example, 2-3 weeks or 2-4 weeks) or for 1-2 months (for example, 4-6 weeks or 4-8 weeks).

The compound of formula (I) or a pharmaceutically acceptable salt, solvate and/or derivative thereof may initially be administered up to 1 day in advance, such as up to 2 days in advance, up to 3 days in advance, up to 5 days in advance, up to 1 week in advance, up to 2 weeks in advance or up to 1 month in advance of a noise or blast which is anticipated to cause acute noise-induced hearing loss, administration which is initiated at any point in advance exposure to a noise or blast which is anticipated to cause acute noise-induced hearing loss will typically continue for up to 2 months after exposure to the noise or blast which is anticipated to cause acute noise-induced hearing loss, such as for up to 1 month after, up to 3 weeks after, up to two weeks after, up to 1 week after, up to 5 days after, up to 3 days after, up to 2 days after, or up to 1 day after.

In one embodiment is provided a compound of formula (I) or a pharmaceutically acceptable salt, solvate and/or derivative thereof for use in preventing or reducing the development of a permanent shift in the auditory threshold, wherein the permanent shift in auditory threshold is reduced by at least 10 dB, such as at least 15 dB, at least 20 dB, at least 30 dB, at least 40 dB, or completely.

### Pharmaceutical Compositions

For use in therapy the compounds of the invention are usually administered as a pharmaceutical composition. The invention also provides a pharmaceutical composition comprising a compound of formula (I), or a pharmaceutically acceptable salt and/or solvate (e.g. salt) and/or derivative thereof, and a pharmaceutically acceptable carrier or excipient.

In one aspect, the present invention further provides methods of making a pharmaceutical composition comprising (e.g admixing) at least one compound of formula (I) or a pharmaceutically acceptable salt and/or solvate, together with one or more pharmaceutically acceptable excipients and optionally other therapeutic or prophylactic agents as described herein.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below. Each excipient must be “acceptable” in the sense of being compatible with the other ingredients of the formulation.

Pharmaceutical compositions containing compounds of the formula (I) or a pharmaceutically acceptable salt and/or solvate can be formulated in accordance with known techniques, see for example, Remington's Pharmaceutical Sciences, Mack Publishing Company, Easton, PA, USA.

In one embodiment, there is provided a pharmaceutical composition comprising a compound of formula (I), or a pharmaceutically acceptable salt and/or solvate (e.g. salt) and/or derivative thereof, for use in the treatment or prevention of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Meniere's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease.

In a further embodiment, there is provided a method for the prophylaxis or treatment of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Meniere's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease, which comprises administering to a subject a pharmaceutical composition comprising a compound of formula (I) or a pharmaceutically acceptable salt and/or solvate (e.g. salt) and/or derivative thereof.

The invention also provides the use of a pharmaceutical composition comprising a compound of formula (I), or a pharmaceutically acceptable salt and/or solvate thereof (e.g. salt) and/or derivative thereof, in the manufacture of a medicament for the treatment or prophylaxis of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Ménière's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease.

Compounds with peripheral availability may be of use in the prophylaxis or treatment of a disease or disorder where peripheral modulation of Kv3.1, Kv3.2, Kv3.3 and/or Kv3.4 is required, such as cardiovascular disease or muscular disorders such as Spinal-Bulbar Muscular Atrophy (SBMA).

The compounds of formula (I) or their pharmaceutically acceptable salts and/or solvates and/or derivatives thereof may be administered by any convenient method, e.g. by oral, parenteral, buccal, sublingual, nasal, rectal or transdermal administration, and the pharmaceutical compositions adapted accordingly. Other possible routes of administration include intratympanic and intracochlear.

The compounds of formula (I) or their pharmaceutically acceptable salts and/or solvates and/or derivatives thereof which are active when given orally can be formulated as liquids or solids, e.g. as syrups, suspensions, emulsions, tablets, capsules or lozenges.

A liquid formulation will generally consist of a suspension or solution of the active ingredient (such as a compound of formula (I) or a pharmaceutically acceptable salt and/or solvate (e.g. salt) and/or derivative thereof) in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient (such as a compound of formula (I) or a pharmaceutically acceptable salt and/or solvate (e.g. salt) and/or derivative thereof) can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient (such as a compound of formula (I) or a pharmaceutically acceptable salt and/or solvate (e.g. salt) and/or derivative thereof) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. In one embodiment the composition is in unit dose form such as a tablet, capsule or ampoule.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) polymers (depending on dose). The film coats of the tablet or capsule typically contain 0-10% (w/w) release-controlling (e.g. delaying) polymers, 0-3% (w/w) pigments, and/or 0-2% (w/w) plasticizers.

The compounds of the formula (I) or a pharmaceutically acceptable salt and/or solvate will generally be presented in unit dosage form and, as such, will typically contain sufficient compound to provide a desired level of biological activity.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 mg to 1000 mg, more suitably 1.0 mg to 500 mg, and such unit doses may be administered more than once a day, for example two or three a day. Such therapy may extend for a number of weeks or months.

The dose provided to a subject will typically be a safe and effective dose, i.e. an amount providing an acceptable balance of desired benefits and undesired side effects. A “safe and effective amount” is intended to include an amount of a compound that is effective to achieve a desirable effect in treatment and/or prophylaxis of a disease-state. A desirable effect is typically clinically significant and/or measurable, for instance in the context of (a) preventing the disease-state from occurring in a mammal, in particular, when such mammal is predisposed to the disease-state but has not yet been diagnosed as having it; (b) inhibiting the disease-state, i.e., slowing or arresting its development; and/or (c) relieving the disease-state, i.e., causing regression of the disease state or a reduction in associated symptoms. The safe and effective amount may be one that is sufficient to achieve the desirable effect either when the compound is administered alone, or alternatively when it is administered in combination with one or more further APIs, which either are further compounds for use of the invention or are different from the compounds for use of the invention.

For avoidance of doubt, a “safe and effective amount” as recited herein can be achieved by any suitable dosage regimen, including but not limited to exemplary dosage regimens described elsewhere herein. Hence, for example, references herein to administering a safe and effective amount of a compound, such as by a particular administration route, include achieving the safe and effective amount via a single dose or by plural doses, such as administered by the specified administration route. For instance, orally administering a safe and effective amount includes both orally administering a single dose and orally administering any plural number of doses, provided that a safe and effective amount is thereby achieved by oral administration.

The compounds of formula (I) or a pharmaceutically acceptable salt and/or solvate and/or derivative thereof can be administered as the sole therapeutic agent or they can be administered in combination therapy with one of more other compounds (or therapies) also suitable for the treatment or prophylaxis of the diseases and disorders listed herein.

The invention provides, in a further aspect, a combination comprising a compound of formula (I) or a pharmaceutically acceptable, salt, solvate and/or derivative thereof (e.g. a combination comprising a compound of formula (I) or a pharmaceutically acceptable derivative thereof) together with a further pharmaceutically acceptable active ingredient or ingredients.

The invention provides a compound of formula (I), for use in combination with a further pharmaceutically acceptable active ingredient or ingredients.

When the compounds are used in combination with other therapeutic agents, the compounds may be administered either sequentially or simultaneously by any convenient route. Alternatively, the compounds may be administered separately.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations. The individual components of combinations may also be administered separately, through the same or different routes.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

Suitably, a compound of formula (I) or a pharmaceutically acceptable, salt, solvate and/or derivative thereof is administered orally.

Suitably, a compound of formula (I) or a pharmaceutically acceptable, salt, solvate and/or derivative thereof is administered at 2 to 400 mg per day, such as 2 to 300 mg per day, especially 5 to 250 mg per day.

Suitably, a compound of formula (I) or a pharmaceutically acceptable, salt, solvate and/or derivative thereof is administered once or twice per day.

Suitably, a compound of formula (I) or a pharmaceutically acceptable, salt, solvate and/or derivative thereof is administered for a period of at least three months.

Desirably, a compound of formula (I) or a pharmaceutically acceptable, salt, solvate and/or derivative thereof is administered orally, once or twice per day, at 2 to 400 mg per day, such as 2 to 300 mg per day, especially 5 to 250 mg per day.

A human subject may be an adult, such as aged 18 to 65. Alternatively, a human subject may be 66 years old or older. A compound of formula (I) or a pharmaceutically acceptable, salt, solvate and/or derivative thereof may be administered to a human subject of less than 18 years of age, such as 4 to 17 years old. Administration to a human subject of less than 18 years of age may be of particular relevance in the context of progressive myoclonic epilepsy and Fragile X syndrome.

For convenience and to assist with patient compliance, delivery technologies such as patches or implants may be used to deliver a compound of formula (I) or a pharmaceutically acceptable, salt, solvate and/or derivative thereof over a sustained period of time e.g. at least one week or at least 4 weeks.

### Experimental

The invention is illustrated by the compounds described below. The following examples describe the laboratory synthesis of specific compounds of the invention and are not meant to limit the scope of the invention in any way with respect to compounds or processes. It is understood that, although specific reagents, solvents, temperatures and time periods are used, there are many possible equivalent alternatives that can be used to produce similar results. This invention is meant to include such equivalents.

### Analytical Equipment

Starting materials, reagents and solvents were obtained from commercial suppliers and used without further purification unless otherwise stated. Unless otherwise stated, all compounds with chiral centres are racemic. Where reactions are described as having been carried out in a similar manner to earlier, more completely described reactions, the general reaction conditions used were essentially the same. Work up conditions used were of the types standard in the art, but may have been adapted from one reaction to another. The starting material may not necessarily have been prepared from the batch referred to. Compounds synthesised may have various purities, ranging from for example 85% to 99%. Calculations of number of moles and yield are in some cases adjusted for this.

HPLC-Mass spectra (HPLC-MS) were taken on an Agilent 1100 Series LC/MSD Mass Spectrometer coupled with HPLC instrument Agilent 1100 Series, operating in positive electrospray ionization mode and in acidic gradient conditions.

Quality Control (8 minutes method): LC/MS-ES+ under acidic conditions was performed on a Phenomenex Luna C18 column (3 μm 2×50 mm). Mobile phase: A: (H₂O+0.05% TFA by vol.)/B: (CH₃CN+0.05% TFA by vol). Gradient: t=0 min 0% (B). From 0 to 95% (B) in 8 min. 95% (B) for 0.5 min. From 95 to 100% (B) in 0.5 min. 100% (B) for 0.5 min. From 100% to 0% (B) in 0.1 min. Stop time 11 min. Column T=40° C. Flow rate: 1.0 ml/min. Mass range ES+: (100-1000 amu, F=60). UV detection wavelengths: DAD 1A=220.8, DAD 1B=254.8. The use of this methodology is indicated by “LC/MS: QC_8_MIN” in the analytic characterization of the described compounds.

Quality Control (3 minutes method): LC/MS-ES+ under acidic conditions was performed on a Zorbax SB C18 column (1.8 μm 3×50 mm). Mobile phase: A: (H₂O+0.05% TFA by vol.)/B: (CH₃CN+0.05% TFA by vol). Gradient: t=0 min 0% (B), from 0 to 95% (B) in 2.5 min, 95% (B) for 0.2 min, from 95 to 100% (B) in 0.2 min, 100% (B) for 0.4 min, from 100% to 0% (B) in 0.1 min. Stop time 4 min. Column T=60° C. Flow rate: 1.5 ml/min. Mass range ES+: (100-1000 amu, F=60). UV detection wavelengths: DAD 1A=220.8, DAD 1B=254.8. The use of this methodology is indicated by “LC/MS: QC_3_MIN” in the analytic characterization of the described compounds.

Proton Magnetic Resonance (NMR) spectra were recorded either on Varian instruments at 300, 400, 500 or 600 MHz, or on Bruker instruments at 400 MHz. Chemical shifts are reported in ppm (δ) using the residual solvent line as internal standard. Splitting patterns are designed as s (singlet), br.s (broad singlet), d (doublet), t (triplet), q (quartet), dd (doublet of doublets), dt (doublet of triplets) and m (multiplet). The NMR spectra were recorded at temperatures ranging from 25 to 60° C.

2D NMR NOESY experiments were acquired with a mixing time of 500 ms using a spectral width of 3355 Hz in both f1 and f2. A total of 256 increments were collected, processed to 1 K with linear prediction, 8 scans each. Data were processed with sine bell shift in both dimensions and with lb=0.3 Hz in f1.

Direct infusion Mass spectra (MS) were run on an Agilent 1100 Series LC/MSD Mass Spectrometer, operating in ES (+) and ES (−) ionization mode [ES (+): Mass range: 100-1000 amu. Infusion solvent: water+0.1% HCO₂H/CH₃CN 50/50. ES (−): Mass range: 100-1000 amu. Infusion solvent: water+0.05% NH₄OH/CH₃CN 50/50]. The use of this methodology is indicated by “MS_1 (ESI)” in the analytic characterization of the described compounds. HPLC-Mass spectra (HPLC-MS) were taken on an Agilent 1100 Series LC/MSD Mass Spectrometer coupled with HPLC instrument Agilent 1100 Series, operating in positive electrospray ionization mode and in acidic gradient conditions.

In a number of preparations, purification was performed using manual flash chromatography, semi-automatic flash chromatography (Biotage Flash Master Personal) or automatic flash chromatography (Biotage SP1, SP4 or Isolera).

Flash chromatographies on silica gel were carried out on pre-packed Biotage silica cartridges (e.g. Biotage SNAP or SFAR cartridge KP-Sil), or pre-packed Modus silica cartridges. Reverse phase C18 Flash Chromatographies were carried out using VARIAN MEGA BE-C18 cartridges, or pre-packed Biotage C18 cartridges (e.g. Biotage SNAP cartridge KP-C18-HS).

### Abbreviations

BOC tert-butyloxycarbonyl
CaCl₂ calcium chloride
CO₂ carbon dioxide
cHex cyclohexane
DCM dichloromethane
DIPEA N,N-diisopropylethylamine
DMA N,N-dimethylacetamide
DMF N,N-dimethylformamide
DMSO dimethylsulfoxide
DMSO-d₆ deutrated dimethylsulfoxide
EtOAc ethyl acetate
EtOH ethanol
h hours
HATU N, N,N′,N′-tetramethyl-1-(3-oxidotriazolo[4,5-b]pyridin-3-ium-1-yl)methanediamine hexafluorophosphate, 1-[Bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide
HCl hydrochloric acid
KCl potassium chloride
KH₂PO₄ potassium phosphate monobasic
MeOH methanol
MgCl·6H2O magnesium chloride hexahydrate
NaCl sodium chloride
NaHCO₃ sodium hydrogen carbonate
Na₃PO₄ sodium phosphate tribasic
Na₂SO₄ sodium sulfate
NMR nuclear magnetic resonance
rt room temperature
TEA triethylamine
TFA trifluoroacetic acid
THE tetrahydrofuran

### Experimental Procedures

### Intermediate 1

### 6-(7-methylspiro[2H-benzofuran-3,1′-cyclopropane]-4-yl)oxy-3-nitro-pyridin-2-amine

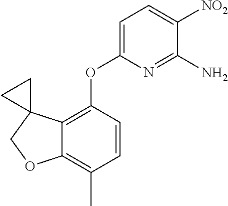 [SMILES: Cc1ccc(Oc2ccc([N+](=O)[O-])c(N)n2)c2c1OCC21CC1]

A mixture of 7-methylspiro[2H-benzofuran-3,1′-cyclopropane]-4-ol (Intermediate 156 WO2012/076877, 500 mg, 2.84 mmol), 6-chloro-3-nitro-pyridin-2-amine (492 mg, 2.84 mmol) and potassium carbonate (588 mg, 4.26 mmol) in N,N-dimethylacetamide (5 mL) was stirred at 60° C. overnight. After cooling, the mixture was diluted with ethyl acetate (50 mL) and washed with brine (50 mL). Phases were separated and the organic layer was dried over Na₂SO₄, filtered and evaporated affording 6-(7-methylspiro[2H-benzofuran-3,1′-cyclopropane]-4-yl)oxy-3-nitro-pyridin-2-amine (889 mg) that was used in the next step without further purification.

LC/MS: QC_3_MIN: Rt=2.51 min; m/z 314 [M+H]⁺.

The following compounds were prepared using the foregoing methodology, replacing 7-methyl spiro[2H-benzofuran-3,1′-cyclopropane]-4-ol with the appropriate phenol:

TABLE 1

| Int. | Structure | Name | Phenol derivative | LC-MS |
|---|---|---|---|---|
| 2 | 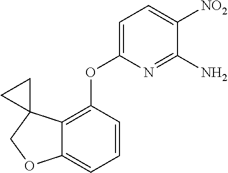 [SMILES: Nc1nc(Oc2cccc3c2C2(CC2)CO3)ccc1[N+](=O)[O-]] | 3-nitro-6-spiro[2H- benzofuran-3,1′- cyclopropane]-4-yloxy- pyridin-2-amine | spiro[2H-benzofuran- 3,1′-cyclopropane]-4-ol (Intermediate 85, WO2012/076877) | LC/MS: QC_3_MIN: Rt = 2.40 min; m/z 300 [M + H]⁺. |
| 3 | 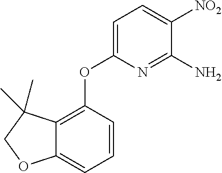 [SMILES: CC1(C)COc2cccc(Oc3ccc([N+](=O)[O-])c(N)n3)c21] | 6-[(3,3-dimethyl-2H- benzofuran-4-yl)oxy]-3- nitro-pyridin-2-amine | 3,3-dimethyl-2H- benzofuran-4-ol (Intermediate 50, WO2012/076877) | LC/MS: QC_3_MIN: Rt = 2.45 min; m/z 302 [M + H]⁺. |
| 4 | 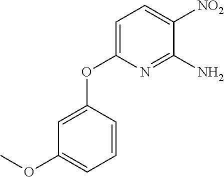 [SMILES: COc1cccc(Oc2ccc([N+](=O)[O-])c(N)n2)c1] | 6-(3-methoxyphenoxy)-3- nitro-pyridin-2-amine | 3-methoxyphenol | LC/MS: QC_3_MIN: Rt = 2.29 min; m/z 262 [M + H]⁺. |
| 5 | 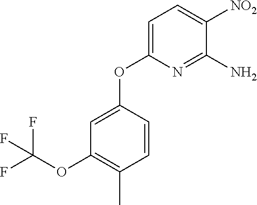 [SMILES: Cc1ccc(Oc2ccc([N+](=O)[O-])c(N)n2)cc1OC(F)(F)F] | 6-[4-methyl-3- (trifluoromethoxy) phenoxy]-3-nitro- pyridin-2-amine | 4-methyl-3- (trifluoromethoxy)phenol (Intermediate 116, WO2011/069951) | LC/MS: QC_3_MIN: Rt = 2.62 min; m/z 330 [M + H]⁺. |
| 6 | 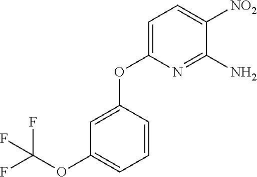 [SMILES: Nc1nc(Oc2cccc(OC(F)(F)F)c2)ccc1[N+](=O)[O-]] | 3-nitro-6-[3- (trifluoromethoxy) phenoxy]pyridin- 2-amine | 3- (trifluoromethoxy)phenol | LC/MS: QC_3_MIN: Rt = 2.51 min; m/z 316 [M + H]⁺. |
| 7 | 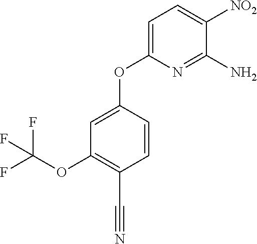 [SMILES: N#Cc1ccc(Oc2ccc([N+](=O)[O-])c(N)n2)cc1OC(F)(F)F] | 4-[(6-amino-5-nitro-2- pyridyl)oxy]-2- (trifluoromethoxy) benzonitrile | 4-hydroxy-2- (trifluoromethoxy) benzonitrile (Intermediate 119, WO2011/069951) | LC/MS: QC_3_MIN: Rt= 2.41 min; m/z 341 [M + H]⁺. |
| 8* | 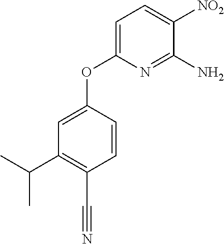 [SMILES: CC(C)c1cc(Oc2ccc([N+](=O)[O-])c(N)n2)ccc1C#N] | 4-[(6-amino-5-nitro-2- pyridyl)oxy]-2-isopropyl- benzonitrile | 4-hydroxy-2- isopropylbenzonitrile | LC/MS: QC_3_MIN: Rt = 2.45 min; m/z 299 [M + H]⁺. |
| 9* | 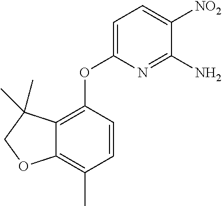 [SMILES: Cc1ccc(Oc2ccc([N+](=O)[O-])c(N)n2)c2c1OCC2(C)C] | 3-nitro-6-[(3,3,7-trimethyl- 2H-benzofuran-4- yl)oxy]pyridin-2-amine | 3,3,7-trimethyl-2H- benzofuran-4-ol (Intermediate 184 WO2012/076877) | LC/MS: QC_3_MIN: Rt = 2.75 min; m/z 316 [M + H]⁺. |

*Reaction time 5 hours

### Intermediate 10

### 6-(7-methylspiro[2H-benzofuran-3,1′-cyclopropane]-4-yl)oxypyridine-2,3-diamine

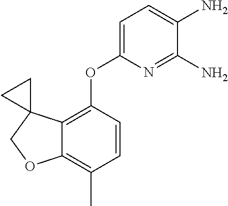 [SMILES: Cc1ccc(Oc2ccc(N)c(N)n2)c2c1OCC21CC1]

### Procedure A

6-(7-methylspiro[2H-benzofuran-3,1′-cyclopropane]-4-yl)oxy-3-nitro-pyridin-2-amine (Intermediate 1, 615 mg, 1.96 mmol), iron (548 mg, 9.81 mmol) and ammonium chloride (525 mg, 9.81 mmol) were suspended in a mixture of ethanol (8 mL)/water (2 mL). The mixture was stirred at 80° C. for 5 hours. After cooling, the solids were filtered off and the filtrate was diluted with EtOAc (30 mL) and washed with water (30 mL) and then with brine (30 mL). Phases were separated and the organic layer was dried over Na₂SO₄, filtered and evaporated. The residue was purified by flash chromatography (Biotage System) on silica gel using a SFAR 5 g as column and Cyclohexane/Ethyl acetate from 95:5 to 0:100 as eluent, affording 6-(7-methylspiro[2H-benzofuran-3,1′-cyclopropane]-4-yl)oxypyridine-2,3-diamine (110 mg) as yellow oil.

LC/MS: QC_3_MIN: Rt=1.92 min; m/z 284 [M+H]⁺.

### Procedure B

6-(7-methylspiro[2H-benzofuran-3,1′-cyclopropane]-4-yl)oxy-3-nitro-pyridin-2-amine (Intermediate 1, 450 mg, 1.44 mmol), iron (401 mg, 7.18 mmol) and ammonium chloride (384 mg, 7.18 mmol) were suspended in a mixture of ethanol (4 mL)/water (3 mL)/tetrahydrofuran (8 mL). The mixture was stirred at 70° C. for 1.5 hours and then at room temperature overnight. The solids were filtered off and the filtrate was diluted with DCM (50 mL) and washed with water (50 mL) and then with brine (50 mL). Phases were separated and the organic layer was dried over Na₂SO₄, filtered and evaporated. The residue was purified by flash chromatography (Biotage System) on silica gel using a SFAR 10 g as column and DCM:methanol from 99:1 to 95:5 as eluent, affording 6-(7-methylspiro[2H-benzofuran-3,1′-cyclopropane]-4-yl)oxypyridine-2,3-diamine (240 mg) as pink solid.

LC/MS: QC_3_MIN: Rt=1.95 min; m/z 284 [M+H]⁺.

The following compounds were prepared using either the foregoing methodology A or B, replacing 6-(7-methylspiro[2H-benzofuran-3,1′-cyclopropane]-4-yl)oxy-3-nitro-pyridin-2-amine (Intermediate 1) with the appropriate nitro-amino-pyridine derivative and were heated between 0.5 and 5 hours. Final products were purified by flash-chromatography (Silica cartridge using Cyclohexane/EtOAc or DCM/Methanol as eluent or C-18 cartridge using water/acetonitrile as eluent).

TABLE 2

| | | | Nitro-amino-pyridine | | |
|---|---|---|---|---|---|
| Int. | Structure | Name | derivative | Procedure | LC-MS |
| 11 | 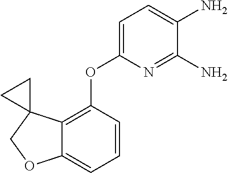 [SMILES: Nc1ccc(Oc2cccc3c2C2(CC2)CO3)nc1N] | 6-spiro[2H- benzofuran-3,1′- cyclopropane]- 4-yloxypyridine- 2,3-diamine | 3-nitro-6-spiro[2H- benzofuran-3,1′- cyclopropane]-4-yloxy- pyridin-2-amine (Intermediate 2) | B (1.5 h) | LC/MS: QC_3_MIN: Rt = 2.12 min; m/z 270 [M + H]⁺. |
| 12 | 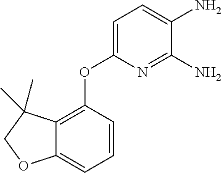 [SMILES: CC1(C)COc2cccc(Oc3ccc(N)c(N)n3)c21] | 6-[(3,3-dimethyl- 2H-benzofuran- 4- yl)oxy]pyridine- 2,3-diamine | 6-[(3,3-dimethyl-2H- benzofuran-4-yl)oxy]- 3-nitro-pyridin-2-amine (Intermediate 3) | A (1 h) | LC/MS: QC_3_MIN: Rt = 1.89 min; m/z 272 [M + H]⁺. |
| 13 | 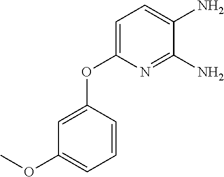 [SMILES: COc1cccc(Oc2ccc(N)c(N)n2)c1] | 6-(3- methoxyphenox y)pyridine-2,3- diamine | 6-(3- methoxyphenoxy)-3- nitro-pyridin-2-amine (Intermediate 4) | A (3 h) | LC/MS: QC_3_MIN: Rt = 1.71 min; m/z 232 [M + H]⁺. |
| 14 | 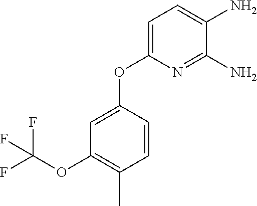 [SMILES: Cc1ccc(Oc2ccc(N)c(N)n2)cc1OC(F)(F)F] | 6-[4-methyl-3- (trifluoromethoxy) phenoxy] pyridine-2,3- diamine | 6-[4-methyl-3- (trifluoromethoxy) phenoxy]-3-nitro- pyridin-2-amine (Intermediate 5) | A (4 h) | LC/MS: QC_3_MIN: Rt = 2.07 min; m/z 300 [M + H]⁺. |
| 15 | 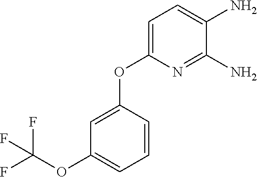 [SMILES: Nc1ccc(Oc2cccc(OC(F)(F)F)c2)nc1N] | 6-[3- (trifluoromethoxy) phenoxy] pyridine-2,3- diamine | 3-nitro-6-[3- (trifluoromethoxy) phenoxy]pyridin- 2-amine (Intermediate 6) | A (4 h) | LC/MS: QC_3_MIN: Rt = 1.99 min; m/z 286 [M + H]⁺. |
| 16 | 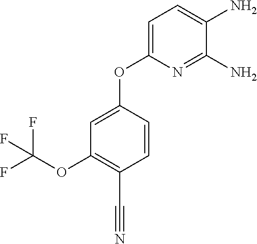 [SMILES: N#Cc1ccc(Oc2ccc(N)c(N)n2)cc1OC(F)(F)F] | 4-[(5,6-diamino- 2-pyridyl)oxy]-2- (trifluoromethoxy) benzonitrile | 4-[(6-amino-5-nitro-2- pyridyl)oxy]-2- (trifluoromethoxy) benzonitrile (Intermediate 7) | A (2 h) | LC/MS: QC_3_MIN: Rt = 1.99 min; m/z 311 [M + H]⁺. |
| 17 | 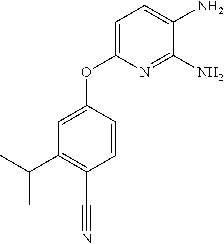 [SMILES: CC(C)c1cc(Oc2ccc(N)c(N)n2)ccc1C#N] | 4-[(5,6-diamino- 2-pyridyl)oxy]-2- isopropyl- benzonitrile | 4-[(6-amino-5-nitro-2- pyridyl)oxy]-2- isopropyl-benzonitrile (Intermediate 8) | A (1 h) | LC/MS: QC_3_MIN: Rt = 1.99 min; m/z 269 [M + H]⁺. |
| 18 | 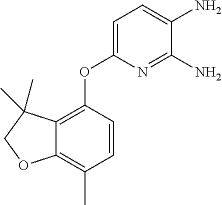 [SMILES: Cc1ccc(Oc2ccc(N)c(N)n2)c2c1OCC2(C)C] | 6-[(3,3,7- trimethyl-2H- benzofuran-4- yl)oxy]pyridine- 2,3-diamine | 3-nitro-6-[(3,3,7- trimethyl-2H- benzofuran-4- yl)oxy]pyridin-2-amine (Intermediate 9) | A (0.5 h) | LC/MS: QC_3_MIN: Rt = 2.11 min; m/z 286 [M + H]⁺. |

### Intermediate 19

### tert-butyl N-[2-[[2-amino-6-(7-methylspiro[2H-benzofuran-3,1′-cyclopropane]-4-yl)oxy-3-pyridyl]amino]-1,1-dimethyl-2-oxo-ethyl]carbamate

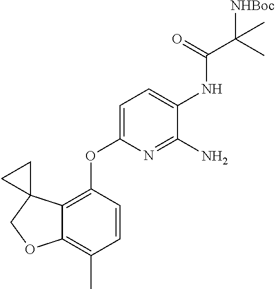 [SMILES: Cc1ccc(Oc2ccc(NC(=O)C(C)(C)NC(=O)OC(C)(C)C)c(N)n2)c2c1OCC21CC1]

To a solution of 2-(tert-butoxycarbonylamino)-2-methyl-propanoic acid (72 mg, 0.35 mmol) in DMF (5 mL), N,N-diisopropylethylamine (185 μL, 1.06 mmol) and N,N,N′,N′-tetramethyl-1-(3-oxidotriazolo[4,5-b]pyridin-3-ium-1-yl)methanediamine hexafluorophosphate (HATU) (148 mg, 0.39 mmol) were added and the mixture was stirred for 10 minutes. Then, a solution of 6-(7-methylspiro[2H-benzofuran-3,1′-cyclopropane]-4-yl)oxypyridine-2,3-diamine (Intermediate 10, 100 mg, 0.35 mmol) in DMF (1 mL) was added and the mixture was stirred at 40° C. overnight. The reaction was quenched with an aqueous saturated solution of NaHCO₃ (20 mL) and diluted with ethyl acetate (20 mL). Phases were separated and the organic layer was washed with brine (20 mL), dried over Na₂SO₄, filtered and then concentrated in vacuo. The residue was purified by flash chromatography on silica gel using a SFAR 5 g as column and cyclohexane: ethyl acetate from 90:10 to 0:100 as eluent; the desired fractions were collected and dried affording tert-butyl N-[2-[[2-amino-6-(7-methylspiro[2H-benzofuran-3,1′-cyclopropane]-4-yl)oxy-3-pyridyl]amino]-1,1-dimethyl-2-oxo-ethyl]carbamate (90 mg) as pale yellow solid.

LC/MS: QC_3_MIN: Rt=2.40 min; m/z 469 [M+H]⁺.

The following compounds were prepared using the foregoing methodology, replacing 6-(7-methylspiro[2H-benzofuran-3,1′-cyclopropane]-4-yl)oxypyridine-2,3-diamine (Intermediate 10) with the appropriate diamine derivative. Final products were purified by flash-chromatography (Silica cartridge; Cyclohexane/EtOAc or other appropriate solvent system).

TABLE 3

| Int. | Structure | Name | Diamine derivative | LC-MS |
|---|---|---|---|---|
| 20 | 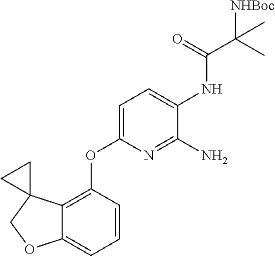 [SMILES: CC(C)(C)OC(=O)NC(C)(C)C(=O)Nc1ccc(Oc2cccc3c2C2(CC2)CO3)nc1N] | tert-butyl N-[2-[(2-amino- 6-spiro[2H-benzofuran- 3, 1′-cyclopropane]-4- yloxy-3-pyridyl)amino]- 1,1-dimethyl-2-oxo- ethyl]carbamate | 6-spiro[2H-benzofuran- 3,1′-cyclopropane]-4- yloxypyridine-2,3-diamine (Intermediate 11) | LC/MS: QC_3_MIN: Rt = 2.35 min; m/z 455 [M + H]⁺. |
| 21 | 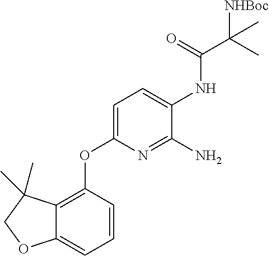 [SMILES: CC(C)(C)OC(=O)NC(C)(C)C(=O)Nc1ccc(Oc2cccc3c2C(C)(C)CO3)nc1N] | tert-butyl N-[2-[[2-amino- 6-[(3,3-dimethyl-2H- benzofuran-4-yl)oxy]-3- pyridyl]amino]-1,1- dimethyl-2-oxo- ethyl]carbamate | 6-[(3,3-dimethyl-2H- benzofuran-4- yl)oxy]pyridine-2,3- diamine (Intermediate 12) | LC/MS: QC_3_MIN: Rt = 2.41 min; m/z 457 [M + H]⁺. |
| 22 | 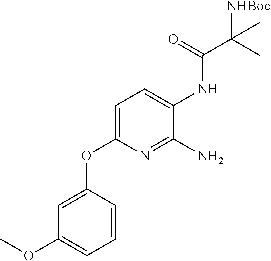 [SMILES: COc1cccc(Oc2ccc(NC(=O)C(C)(C)NC(=O)OC(C)(C)C)c(N)n2)c1] | tert-butyl N-[2-[2-amino- 6-(3-methoxyphenoxy)-3- pyridyl]amino]-1,1- dimethyl-2-oxo- ethyl]carbamate | 6-(3- methoxyphenoxy)pyridine- 2,3-diamine (Intermediate 13) | LC/MS: QC_3_MIN: Rt = 2.24 min; m/z 417 [M + H]⁺ |
| 23 | 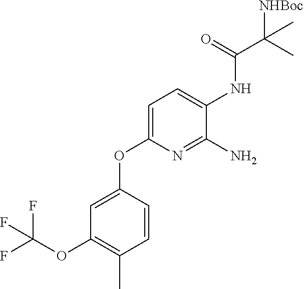 [SMILES: Cc1ccc(Oc2ccc(NC(=O)C(C)(C)NC(=O)OC(C)(C)C)c(N)n2)cc1OC(F)(F)F] | tert-butyl N-[2-[2-amino- 6-[4-methyl-3- (trifluoromethoxy) phenoxy]-3-pyridyl] amino]-1,1- dimethyl-2-oxo- ethyl]carbamate | 6-[4-methyl-3- (trifluoromethoxy) phenoxy]pyridine- 2,3-diamine (Intermediate 14) | LC/MS: QC_3_MIN: Rt = 2.52 min; m/z 485 [M + H]⁺. |
| 24 | 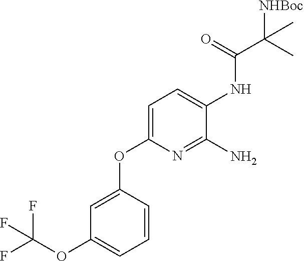 [SMILES: CC(C)(C)OC(=O)NC(C)(C)C(=O)Nc1ccc(Oc2cccc(OC(F)(F)F)c2)nc1N] | tert-butyl N-[2-[[2-amino- 6-[3- (trifluoromethoxy) phenoxy]-3-pyridyl] amino]-1,1- dimethyl-2-oxo- ethyl]carbamate | 6-[3- (trifluoromethoxy) phenoxy]pyridine- 2,3-diamine (Intermediate 15) | LC/MS: QC_3_MIN: Rt = 2.47 min; m/z 471 [M + H]⁺. |
| 25 | 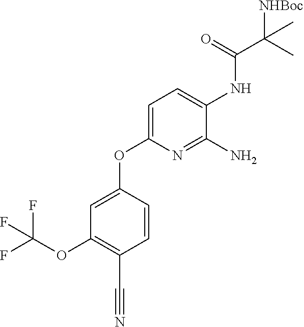 [SMILES: CC(C)(C)OC(=O)NC(C)(C)C(=O)Nc1ccc(Oc2ccc(C#N)c(OC(F)(F)F)c2)nc1N] | tert-butyl N-[2-[2-amino- 6-[4-cyano-3- (trifluoromethoxy) phenoxy]-3-pyridyl] amino]-1,1- dimethyl-2-oxo- ethyl]carbamate | 4-[(5,6-diamino-2- pyridyl)oxy]-2- (trifluoromethoxy) benzonitrile (Intermediate 16) | LC/MS: QC_3_MIN: Rt = 2.40 min; m/z 496 [M + H]⁺. |
| 26 | 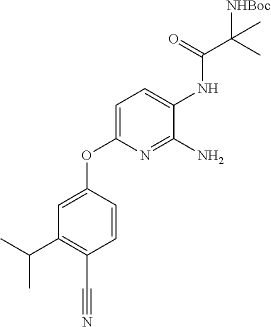 [SMILES: CC(C)c1cc(Oc2ccc(NC(=O)C(C)(C)NC(=O)OC(C)(C)C)c(N)n2)ccc1C#N] | tert-butyl N-[2-[[2-amino- 6-(4-cyano-3-isopropyl- phenoxy)-3- pyridyl]amino]-1,1- dimethyl-2-oxo- ethyl]carbamate | 4-[(5,6-diamino-2- pyridyl)oxy]-2-isopropyl- benzonitrile (Intermediate 17) | LC/MS: QC_3_MIN: Rt = 2.69 min; m/z 454 [M + H]⁺. |
| 27 | 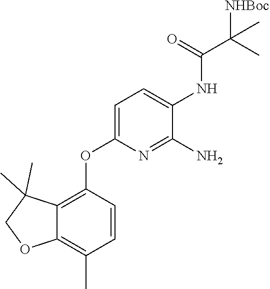 [SMILES: Cc1ccc(Oc2ccc(NC(=O)C(C)(C)NC(=O)OC(C)(C)C)c(N)n2)c2c1OCC2(C)C] | tert-butyl N-[2-[2-amino- 6-[(3,3,7-trimethyl-2H- benzofuran-4-yl)oxy]-3- pyridyl]amino]-1,1- dimethyl-2-oxo- ethyl]carbamate | 6-[(3,3,7-trimethyl-2H- benzofuran-4- yl)oxy]pyridine-2,3- diamine (Intermediate 18) | LC/MS: QC_3_MIN: Rt = 2.57 min; m/z 472 [M + H]⁺. |

### Intermediate 28

### Tert-butyl N-[(1R)-1-[[2-amino-6-(7-methylspiro[2H-benzofuran-3,1′-cyclopropane]-4-yl)oxy-3-pyridyl]carbamoyl]propyl]carbamate

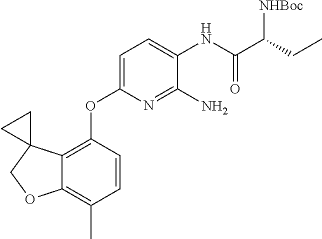 [SMILES: CC[C@@H](NC(=O)OC(C)(C)C)C(=O)Nc1ccc(Oc2ccc(C)c3c2C2(CC2)CO3)nc1N]

To a solution of (R)-2-((tert-butoxycarbonyl)amino)butanoic acid (36 mg, 0.18 mmol) in DMF (5 mL), N,N-diisopropylethylamine (90 NL, 0.53 mmol) and N,N,N′,N′-tetramethyl-1-(3-oxidotriazolo[4,5-b]pyridin-3-ium-1-yl)methanediamine hexafluorophosphate (HATU) (74 mg, 0.19 mmol) were added and the mixture was stirred for 10 minutes. Then, a solution of 6-(7-methyl spiro[2H-benzofuran-3,1′-cyclopropane]-4-yl)oxypyridine-2,3-diamine (Intermediate 10, 50 mg, 0.18 mmol) in DMF (1 mL) was added and the mixture was stirred at room temperature for 2 hours. The reaction was quenched with a saturated solution of NaHCO₃ (20 mL) and diluted with ethyl acetate (20 mL). Phases were separated and the organic layer was washed with brine (20 mL), dried over Na₂SO₄, filtered, then concentrated in vacuo. The residue was purified by flash chromatography (Biotage system) on silica gel using a SFAR 5 g as column and cyclohexane:ethyl acetate from 90:10 to 40:60 as eluent; the desired fractions were collected and dried affording tert-butyl N-[(1R)-1-[[2-amino-6-(7-methylspiro[2H-benzofuran-3,1′-cyclopropane]-4-yl)oxy-3-pyridyl]carbamoyl]propyl]carbamate (60 mg,) as pale pink solid.

LC/MS: QC_3_MIN: Rt=2.44 min; m/z 469 [M+H]⁺.

The following compounds were prepared using the foregoing methodology, replacing 6-(7-methylspiro[2H-benzofuran-3,1′-cyclopropane]-4-yl)oxypyridine-2,3-diamine (Intermediate 10) with the appropriate diamine derivative. The reactions were performed at different heating conditions with temperature ranging from room temperature to 40° C. The reaction conditions for each case are specified in the Table. Final products were purified by flash-chromatography (Silica cartridge; Cyclohexane/EtOAc or other appropriate solvent system).

TABLE 4

| | | | | Reaction | |
|---|---|---|---|---|---|
| Int. | Structure | Name | Diamine derivative | conditions | LC-MS |
| 29 | 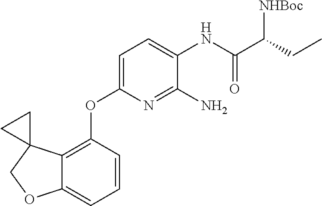 [SMILES: CC[C@@H](NC(=O)OC(C)(C)C)C(=O)Nc1ccc(Oc2cccc3c2C2(CC2)CO3)nc1N] | tert-butyl N-[(1R)-1- [(2-amino-6- spiro[2H- benzofuran-3,1′- cyclopropane]-4- yloxy-3- pyridyl)carbamoyl] propyl]carbamate | 6-spiro[2H- benzofuran-3,1′- cyclopropane]-4- yloxypyridine-2,3- diamine (Intermediate 11) | 40° C., overnight | LC/MS: QC_3_MIN: Rt = 2.32 min; m/z 455 [M + H]⁺. |
| 30 | 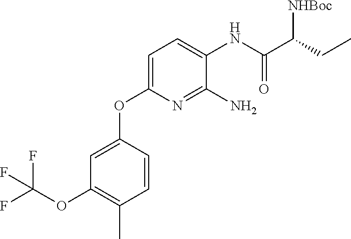 [SMILES: CC[C@@H](NC(=O)OC(C)(C)C)C(=O)Nc1ccc(Oc2ccc(C)c(OC(F)(F)F)c2)nc1N] | tert-butyl N-[(1R)-1- [[2-amino-6-[4- methyl-3- (trifluoromethoxy) phenoxy]-3- pyridyl]carbamoyl] propyl]carbamate | 6-[4-methyl-3- (trifluoromethoxy) phenoxy]pyridine- 2,3-diamine (Intermediate 14) | 40° C., overnight | LC/MS: QC_3_MIN: Rt = 2.56 min; m/z 485 [M + H]⁺. |
| 31 | 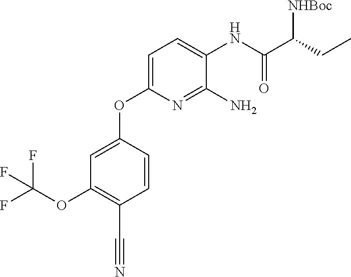 [SMILES: CC[C@@H](NC(=O)OC(C)(C)C)C(=O)Nc1ccc(Oc2ccc(C#N)c(OC(F)(F)F)c2)nc1N] | tert-butyl N-[(1R)-1- [[2-amino-6-[4- cyano-3- (trifluoromethoxy) phenoxy]-3- pyridyl]carbamoyl] propyl]carbamate | 4-[(5,6-diamino-2- pyridyl)oxy]-2- (trifluoromethoxy) benzonitrile (Intermediate 16) | rt, overnight | LC/MS: QC_3_MIN: Rt =2.43 min; m/z 496 [M + H]⁺. |
| 32 | 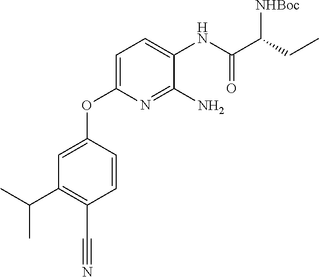 [SMILES: CC[C@@H](NC(=O)OC(C)(C)C)C(=O)Nc1ccc(Oc2ccc(C#N)c(C(C)C)c2)nc1N] | tert-butyl N-[(1R)-1- [[2-amino-6-(4- cyano-3-isopropyl- phenoxy)-3- pyridyl]carbamoyl] propyl]carbamate | 4-[(5,6-diamino-2- pyridyl)oxy]-2- isopropyl- benzonitrile (Intermediate 17) | rt, overnight | LC/MS: QC_3_MIN: Rt = 2.59 min; m/z 454 [M + H]⁺. |

### Intermediate 33

### 2-amino-N-[2-amino-6-(7-methylspiro[2H-benzofuran-3,1′-cyclopropane]-4-yl)oxy-3-pyridyl]-2-methyl-propanamide

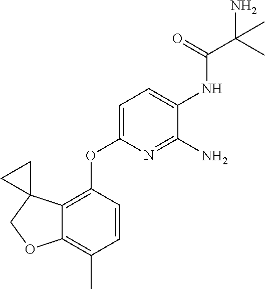 [SMILES: Cc1ccc(Oc2ccc(NC(=O)C(C)(C)N)c(N)n2)c2c1OCC21CC1]

To a solution of tert-butyl N-[2-[[2-amino-6-(7-methylspiro[2H-benzofuran-3,1′-cyclopropane]-4-yl)oxy-3-pyridyl]amino]-1,1-dimethyl-2-oxo-ethyl]carbamate (Intermediate 19, 90 mg, 0.19 mmol) in dichloromethane (5 mL) at 0° C., trifluoroacetic acid (1 mL) was added and the reaction mixture was stirred for 3 hours while the temperature was allowed to reach room temperature. The reaction was quenched with water (5 mL), diluted with dichloromethane (20 mL) and an aqueous saturated solution of NaHCO₃ was added while the pH was allowed to reach ~8; phases were separated and the organic layer was dried over Na₂SO₄, filtered and evaporated affording 2-amino-N-[2-amino-6-(7-methylspiro[2H-benzofuran-3,1′-cyclopropane]-4-yl)oxy-3-pyridyl]-2-methyl-propanamide (70 mg) as pale pink solid that was used in the next step without further purification.

LC/MS: QC_3_MIN: Rt=1.95 min; m/z 369 [M+H]⁺.

The following compounds were prepared using the foregoing methodology, replacing tert-butyl N-[2-[[2-amino-6-(7-methylspiro[2H-benzofuran-3,1′-cyclopropane]-4-yl)oxy-3-pyridyl]amino]-1,1 dimethyl-2-oxo-ethyl]carbamate (Intermediate 19) with the appropriate Boc derivative and for a reaction time between 2 and 4 hours.

TABLE 5

| Int. | Structure | Name | Boc derivative | LC-MS |
|---|---|---|---|---|
| 34 | 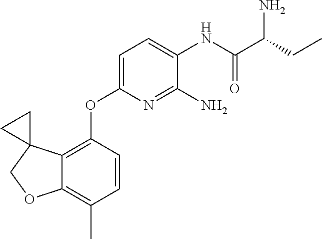 [SMILES: CC[C@@H](N)C(=O)Nc1ccc(Oc2ccc(C)c3c2C2(CC2)CO3)nc1N] | (2R)-2-amino-N-[2- amino-6-(7- methylspiro[2H- benzofuran-3,1′- cyclopropane]-4-yl)oxy- 3-pyridyl]butanamide | tert-butyl N-[(1R)-1-[2- amino-6-(7- methylspiro[2H- benzofuran-3,1′- cyclopropane]-4-yl)oxy-3- pyridyl]carbamoyl]propyl] carbamate (Intermediate 28) | LC/MS: QC_3_MIN: Rt = 1.94 min; m/z 369 [M + H]⁺. |
| 35 | 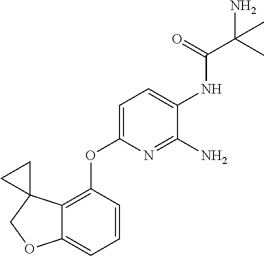 [SMILES: CC(C)(N)C(=O)Nc1ccc(Oc2cccc3c2C2(CC2)CO3)nc1N] | 2-amino-N-(2-amino-6- spiro[2H-benzofuran- 3,1′-cyclopropane]-4- yloxy-3-pyridyl)-2- methyl-propanamide | tert-butyl N-[2-[(2-amino- 6-spiro[2H-benzofuran- 3,1′-cyclopropane]-4- yloxy-3-pyridyl)amino]- 1,1-dimethyl-2-oxo- ethyl]carbamate (Intermediate 20) | LC/MS: QC_3_MIN: Rt = 1.93 min; m/z 355 [M + H]⁺. |
| 36 | 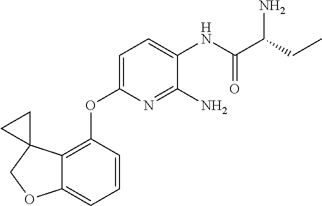 [SMILES: CC[C@@H](N)C(=O)Nc1ccc(Oc2cccc3c2C2(CC2)CO3)nc1N] | (2R)-2-amino-N-(2- amino-6-spiro[2H- benzofuran-3,1′- cyclopropane]-4-yloxy- 3-pyridyl)butanamide | tert-butyl N-[(1R)-1-[(2- amino-6-spiro[2H- benzofuran-3,1′- cyclopropane]-4-yloxy-3- pyridyl)carbamoyl]propyl] carbamate (Intermediate 29) | LC/MS: QC_3_MIN: Rt = 1.90 min; m/z 355 [M + H]⁺. |
| 37 | 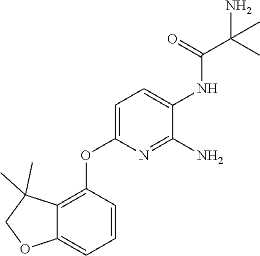 [SMILES: CC(C)(N)C(=O)Nc1ccc(Oc2cccc3c2C(C)(C)CO3)nc1N] | 2-amino-N-[2-amino-6- [(3,3-dimethyl-2H- benzofuran-4-yl)oxy]-3- pyridyl]-2-methyl- propanamide | tert-butyl N-[2-[[2-amino- 6-[(3,3-dimethyl-2H- benzofuran-4-yl)oxy]-3- pyridyl]amino]-1,1- dimethyl-2-oxo- ethyl]carbamate (Intermediate 21) | LC/MS: QC_3_MIN: Rt = 1.90 min; m/z 357 [M + H]⁺. |
| 38 | 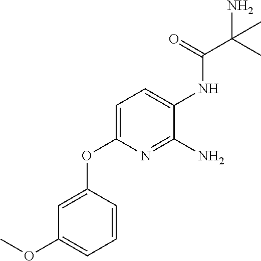 [SMILES: COc1cccc(Oc2ccc(NC(=O)C(C)(C)N)c(N)n2)c1] | 2-amino-N-[2-amino-6- (3-methoxyphenoxy)-3- pyridyl]-2-methyl- propanamide | tert-butyl N-[2-[[2-amino- 6-(3-methoxyphenoxy)-3- pyridyl]amino]-1,1- dimethyl-2-oxo- ethyl]carbamate (Intermediate 22) | LC/MS: QC_3_MIN: Rt = 1.79 min; m/z 317 [M + H]⁺. |
| 39 | 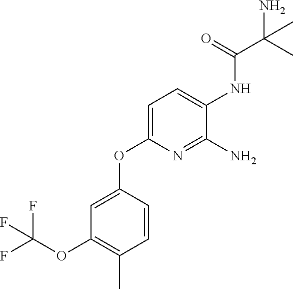 [SMILES: Cc1ccc(Oc2ccc(NC(=O)C(C)(C)N)c(N)n2)cc1OC(F)(F)F] | 2-amino-N-[2-amino-6- [4-methyl-3- (trifluoromethoxy) phenoxy]-3-pyridyl]-2- methyl-propanamide | tert-butyl N-[2-[[2-amino- 6-[4-methyl-3- (trifluoromethoxy) phenoxy]-3-pyridyl] amino]-1,1- dimethyl-2-oxo- ethyl]carbamate (Intermediate 23) | LC/MS: QC_3_MIN: Rt = 2.05 min; m/z 385 [M + H]⁺. |
| 40 | 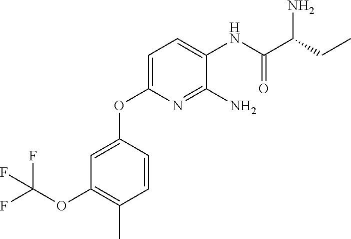 [SMILES: CC[C@@H](N)C(=O)Nc1ccc(Oc2ccc(C)c(OC(F)(F)F)c2)nc1N] | (2R)-2-amino-N-[2- amino-6-[4-methyl-3- (trifluoromethoxy) phenoxy]-3- pyridyl]butanamide | tert-butyl N-[(1R)-1-[2- amino-6-[4-methyl-3- (trifluoromethoxy) phenoxy]-3- pyridyl]carbamoyl]propyl] carbamate (Intermediate 30) | LC/MS: QC_3_MIN: Rt = 2.12 min; m/z 385 [M + H]⁺. |
| 41 | 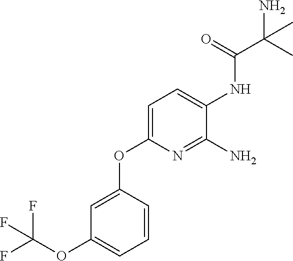 [SMILES: CC(C)(N)C(=O)Nc1ccc(Oc2cccc(OC(F)(F)F)c2)nc1N] | 2-amino-N-[2-amino-6- [3-(trifluoromethoxy) phenoxy]-3-pyridyl]-2- methyl-propanamide | tert-butyl N-[2-[[2-amino- 6-[3- (trifluoromethoxy) phenoxy]-3-pyridyl] amino]-1,1- dimethyl-2-oxo- ethyl]carbamate (Intermediate 24) | LC/MS: QC_3_MIN: Rt = 1.98 min; m/z 371 [M + H]⁺. |
| 42 | 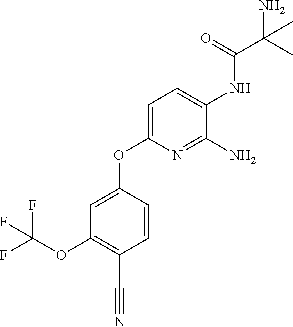 [SMILES: CC(C)(N)C(=O)Nc1ccc(Oc2ccc(C#N)c(OC(F)(F)F)c2)nc1N] | 2-amino-N-[2-amino-6- [4-cyano-3- (trifluoromethoxy) phenoxy]-3-pyridyl]-2- methyl-propanamide | tert-butyl N-[2-[[2-amino- 6-[4-cyano-3- (trifluoromethoxy) phenoxy]-3-pyridyl] amino]-1,1- dimethyl-2-oxo- ethyl]carbamate (Intermediate 25) | LC/MS: QC_3_MIN: Rt = 1.94 min; m/z 396 [M + H]⁺. |
| 43 | 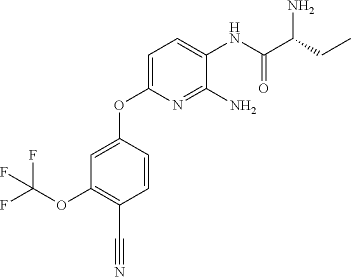 [SMILES: CC[C@@H](N)C(=O)Nc1ccc(Oc2ccc(C#N)c(OC(F)(F)F)c2)nc1N] | (2R)-2-amino-N-[2- amino-6-[4-cyano-3- (trifluoromethoxy) phenoxy]-3- pyridyl]butanamide | tert-butyl N-[(1R)-1-[[2- amino-6-[4-cyano-3- (trifluoromethoxy) phenoxy]-3- pyridyl]carbamoyl]propyl] carbamate (Intermediate 31) | LC/MS: QC_3_MIN: Rt =2.30 min; m/z 396 [M + H]⁺. |
| 44 | 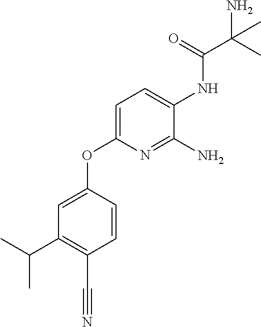 [SMILES: CC(C)c1cc(Oc2ccc(NC(=O)C(C)(C)N)c(N)n2)ccc1C#N] | 2-amino-N-[2-amino-6- (4-cyano-3-isopropyl- phenoxy)-3-pyridyl]-2- methyl-propanamide | tert-butyl N-[2-[[2-amino- 6-(4-cyano-3-isopropyl- phenoxy)-3- pyridyl]amino]-1,1- dimethyl-2-oxo- ethyl]carbamate (Intermediate 26) | LC/MS: QC_3_MIN: Rt = 2.28 min; m/z 354 [M + H]⁺. |
| 45 | 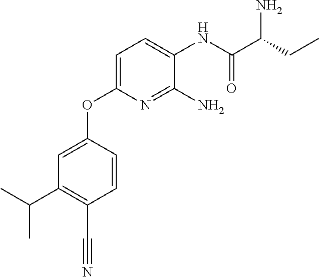 [SMILES: CC[C@@H](N)C(=O)Nc1ccc(Oc2ccc(C#N)c(C(C)C)c2)nc1N] | (2R)-2-amino-N-[2- amino-6-(4-cyano-3- isopropyl-phenoxy)-3- pyridyl]butanamide | tert-butyl N-[(1R)-1-[2- amino-6-(4-cyano-3- isopropyl-phenoxy)-3- pyridyl]carbamoyl]propyl] carbamate (Intermediate 32) | LC/MS: QC_3_MIN: Rt = 2.14 min; m/z 354 [M + H]⁺. |
| 46 | 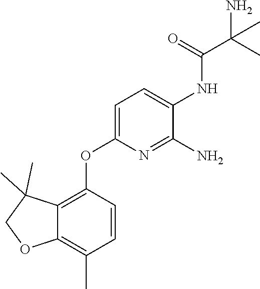 [SMILES: Cc1ccc(Oc2ccc(NC(=O)C(C)(C)N)c(N)n2)c2c1OCC2(C)C] | 2-amino-N-[2-amino-6- [(3,3,7-trimethyl-2H- benzofuran-4-yl)oxy]-3- pyridyl]-2-methyl- propanamide | tert-butyl N-[2-[[2-amino- 6-[(3,3,7-trimethyl-2H- benzofuran-4-yl)oxy]-3- pyridyl]amino]-1,1- dimethyl-2-oxo- ethyl]carbamate (Intermediate 27) | LC/MS: QC_3_MIN: Rt = 2.17 min; m/z 372 [M + H]⁺. |

### Example 1

### 3-[2-amino-6-(7-methylspiro[2H-benzofuran-3,1′-cyclopropane]-4-yl)oxy-3-pyridyl]-5,5-dimethyl-imidazolidine-2,4-dione

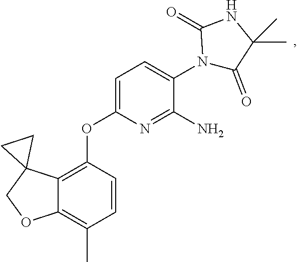 [SMILES: Cc1ccc(Oc2ccc(N3C(=O)NC(C)(C)C3=O)c(N)n2)c2c1OCC21CC1]

To a solution of 2-amino-N-[2-amino-6-(7-methylspiro[2H-benzofuran-3,1′-cyclopropane]-4-yl)oxy-3-pyridyl]-2-methyl-propanamide (Intermediate 33, 22 mg, 0.060 mmol) and triethylamine (20 μL, 0.15 mmol) in dichloromethane (4 mL) at 0° C., a solution of triphosgene (14 mg, 0.048 mmol) in dichloromethane (8 mL) was added and the reaction mixture was stirred for 1 hour while the temperature was allowed to reach room temperature. The reaction was diluted with DCM (30 mL) and washed with brine (20 mL); phases were separated and the organic layer was dried over sodium sulphate, filtered and evaporated to dryness. The residue was purified by reverse flash chromatography (Biotage System) on C-18 phase using a SFAR 12 g as column and Water:Acetonitrile from 95:5 to 5:95 as eluent affording 3-[2-amino-6-(7-methylspiro[2H-benzofuran-3,1′-cyclopropane]-4-yl)oxy-3-pyridyl]-5,5-dimethyl-imidazolidine-2,4-dione (10 mg) as white solid.

The compound was further purified by flash chromatography (Biotage System) on silica gel using a SFAR 5 g as column and Cyclohexane:Ethyl acetate from 75:25 to 0:100 as eluent affording 3-[2-amino-6-(7-methylspiro[2H-benzofuran-3,1-cyclopropane]-4-yl)oxy-3-pyridyl]-5,5-dimethyl-imidazolidine-2,4-dione (4.3 mg) as white solid.

LC/MS: QC_3_MIN: Rt=2.24 min; m/z 395 [M+H]⁺.

¹H-NMR (500 MHz; DMSO-d6): δ ppm 8.36 (bs, 1H), 7.29 (d, 1H), 6.89 (d, 1H), 6.40 (d, 1H), 6.21-6.11 (m, 2H), 5.95 (d, 1H), 4.43 (s, 2H), 2.12 (s, 3H), 1.56-1.27 (m, 6H), 1.24-1.16 (m, 2H), 0.92-0.84 (m, 2H).

The following compounds were prepared using the foregoing methodology, replacing 2-amino-N-[2-amino-6-(7-methylspiro[2H-benzofuran-3,1′-cyclopropane]-4-yl)oxy-3-pyridyl]-2-methyl-propanamide (Intermediate 33) with the appropriate aminoamide derivative and using between 0.4 and 0.8 equivalents of triphosgene.

Final products were purified by flash-chromatography (Silica cartridge using Cyclohexane/EtOAc or DCM/Methanol as eluent or C-18 cartridge using water/acetonitrile as eluent).

TABLE 6

| | | | Aminoamide | | |
|---|---|---|---|---|---|
| Ex. | Structure | Name | derivative | LC-MS | ¹H-NMR |
| 2 | 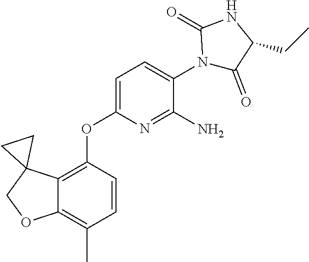 [SMILES: CC[C@H]1NC(=O)N(c2ccc(Oc3ccc(C)c4c3C3(CC3)CO4)nc2N)C1=O] | (5R)-3-[2-amino- 6-(7- methylspiro[2H- benzofuran-3,1′- cyclopropane]- 4-yl)oxy-3- pyridyl]-5-ethyl- imidazolidine- 2,4-dione | (2R)-2-amino-N- [2-amino-6-(7- methylspiro [2H- benzofuran-3,1′- cyclopropane]-4- yl)oxy-3-pyridyl] butanamide (Intermediate 34) | LC/MS: QC_3_ MIN: Rt = 2.25 min; m/z 395 [M + H]⁺. | ¹H-NMR (500 MHz; DMSO-d6): δ ppm 8.47-8.27 (m, 1H), 7.36-7.12 (m, 1H), 6.89 (dd, 1H), 6.39 (d, 1H), 6.35-5.87 (m, 3H), 4.43 (s, 2H), 4.12-4.01 (m, 1H), 2.12 (s, 3H), 1.89-1.55 (m, 2H), 1.25-1.16 (m, 2H), 1.02-0.81 (m, 5H). |
| 3 | 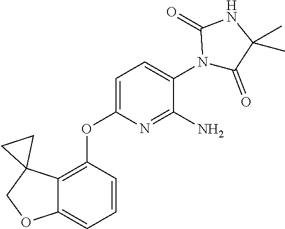 [SMILES: CC1(C)NC(=O)N(c2ccc(Oc3cccc4c3C3(CC3)CO4)nc2N)C1=O] | 3-(2-amino-6- spiro[2H- benzofuran-3,1′- cyclopropane]- 4-yloxy-3- pyridyl)-5,5- dimethyl- imidazolidine- 2,4-dione | 2-amino-N-(2- amino-6-spiro[2H- benzofuran-3,1′- cyclopropane]-4- yloxy-3-pyridyl)-2- methyl- propanamide (Intermediate 35) | LC/MS: QC_3_ MIN: Rt = 2.15 min; m/z 381 [M + H]⁺. | ¹H-NMR (500 MHz; DMSO-d6): δ ppm 8.38 (bs, 1H), 7.32 (d, 1H), 7.09-7.02 (m, 1H), 6.63 (d, 1H), 6.49 (d, 1H), 6.20 (bs, 2H), 6.01 (d, 1H), 4.45 (s, 2H), 1.51-1.30 (m, 6H), 1.30-1.21 (m, 2H), 0.99-0.86 (m, 2H). |
| 4 | 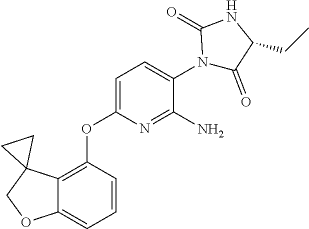 [SMILES: CC[C@H]1NC(=O)N(c2ccc(Oc3cccc4c3C3(CC3)CO4)nc2N)C1=O] | (5R)-3-(2- amino-6- spiro [2H- benzofuran-3,1′- cyclopropane]- 4-yloxy-3- pyridyl)-5-ethyl- imidazolidine- 2,4-dione | (2R)-2-amino-N- (2-amino-6- spiro [2H- benzofuran-3,1′- cyclopropane]-4- yloxy-3-pyridyl) butanamide (Intermediate 36) | LC/MS: QC_3_ MIN: Rt = 2.12 min; m/z 381 [M + H]⁺. | ¹H-NMR (500 MHz; DMSO-d6): δ ppm 8.45-8.24 (m, 1H), 7.31-7.11 (m, 1H), 7.02 (dd, 1H), 6.58 (d, 1H), 6.44 (d, 1H), 6.37-5.87 (m, 3H), 4.41 (s, 2H), 4.13-3.95 (m, 1H), 1.88-1.51 (m, 2H), 1.26-1.17 (m, 2H), 1.01-0.79 (m, 5H). |
| 5 | 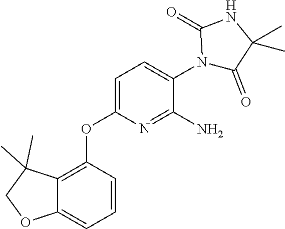 | 3-[2-amino-6- [(3,3-dimethyl- 2H-benzofuran- 4-yl)oxy]-3- pyridyl]-5,5- dimethyl- imidazolidine- 2,4-dione | 2-amino-N-[2- amino-6-[(3,3- dimethyl-2H- benzofuran- 4- yl)oxy]-3-pyridyl]- 2-methyl- propanamide (Intermediate 37) | LC/MS: QC_3_ MIN: Rt = 2.20 min; m/z 383 [M + H]⁺. | ¹H-NMR (500 MHz; DMSO-d6): δ ppm 8.34 (bs, 1H), 7.29 (d, 1H), 7.08 (dd, 1H), 6.57 (dd, 1H), 6.50 (dd, 1H), 6.15 (bs, 2H), 6.01 (d, 1H), 4.17 (s, 2H), 1.50-1.23 (m, 12H). |
| 6 | 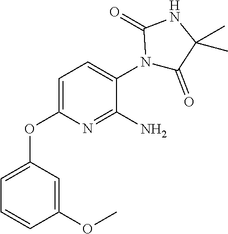 [SMILES: COc1cccc(Oc2ccc(N3C(=O)NC(C)(C)C3=O)c(N)n2)c1] | 3-[2-amino-6-(3- methoxyphenoxy)- 3-pyridyl]-5,5- dimethyl- imidazolidine- 2,4-dione | 2-amino-N-[2- amino-6-(3- methoxyphenoxy)- 3-pyridyl]-2- methyl- propanamide (Intermediate 38) | LC/MS: QC_3_ MIN: Rt = 2.07 min; m/z 343 [M + H]⁺. | ¹H-NMR (500 MHz; DMSO-d6): δ ppm 8.33 (bs, 1H), 7.33- 7.22 (m, 2H), 6.78- 6.62 (m, 3H), 6.11 (bs, 2H), 6.03 (d, 1H), 3.72 (s, 3H), 1.61-1.11 (m, 6H). |
| 7 | 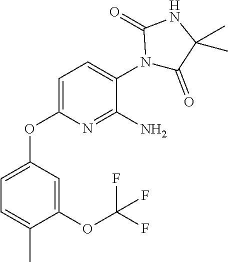 [SMILES: Cc1ccc(Oc2ccc(N3C(=O)NC(C)(C)C3=O)c(N)n2)cc1OC(F)(F)F] | 3-[2-amino-6-[4- methyl-3- (trifluoromethoxy) phenoxy]-3- pyridyl]-5,5- dimethyl- imidazolidine- 2,4-dione | 2-amino-N-[2- amino-6-[4- methyl-3- (trifluoromethoxy) phenoxy]-3- pyridyl]-2-methyl- propanamide (Intermediate 39) | LC/MS: QC_3_ MIN: Rt = 2.35 min; m/z 411 [M + H]⁺. | ¹H-NMR (500 MHz; DMSO-d6): δ ppm 8.34 (s, 1H), 7.38 (d, 1H), 7.32 (d, 1H), 7.08-7.05 (m, 2H), 6.13 (bs, 2H), 6.09 (d, 1H), 2.23 (s, 3H), 1.50-1.26 (m, 6H). |
| 8 | 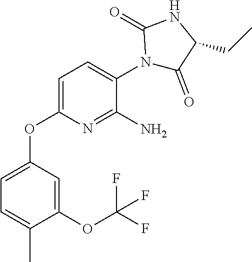 [SMILES: CC[C@H]1NC(=O)N(c2ccc(Oc3ccc(C)c(OC(F)(F)F)c3)nc2N)C1=O] | (5R)-3-[2-amino- 6-[4-methyl-3- (trifluoromethoxy) phenoxy]-3- pyridyl]-5-ethyl- imidazolidine- 2,4-dione | (2R)-2-amino-N- [2-amino-6-[4- methyl-3- (trifluoromethoxy) phenoxy]-3- pyridyl] butanamide (Intermediate 40) | LC/MS: QC_3_ MIN: Rt = 2.35 min; m/z 411 [M + H]⁺. | ¹H-NMR (500 MHz; DMSO-d6): δ ppm 8.47-8.20 (m, 1H), 7.60-6.87 (m, 4H), 6.49 (bs, 2H), 6.20- 5.85 (m, 1H), 4.33- 3.85 (m, 1H), 2.18 (s, 3H), 1.91-1.43 (m, 2H), 0.90 (m, 3H). |
| 9 | 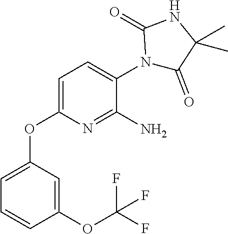 [SMILES: CC1(C)NC(=O)N(c2ccc(Oc3cccc(OC(F)(F)F)c3)nc2N)C1=O] | 3-[2-amino-6-[3- (trifluoromethoxy) phenoxy]-3- pyridyl]-5,5- dimethyl- imidazolidine- 2,4-dione | 2-amino-N-[2- amino-6-[3- (trifluoromethoxy) phenoxy]-3- pyridyl]-2-methyl- propanamide (Intermediate 41) | LC/MS: QC_3 MIN: Rt = 2.39 min; m/z 397 [M + H]⁺. | ¹H-NMR (500 MHz; DMSO-d6): δ ppm 8.37 (bs, 1H), 7.57- 7.49 (m, 1H), 7.38 (d, 1H), 7.21-7.13 (m, 3H), 6.20 (bs, 2H), 6.16 (d, 1H), 1.51-1.30 (m, 6H). |
| 10 | 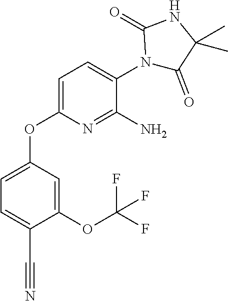 [SMILES: CC1(C)NC(=O)N(c2ccc(Oc3ccc(C#N)c(OC(F)(F)F)c3)nc2N)C1=O] | 4-[[6-amino-5- (4,4-dimethyl- 2,5-dioxo- imidazolidin-1- yl)-2- pyridyl]oxy]-2- (trifluoromethoxy) benzonitrile | 2-amino-N-[2- amino-6-[4-cyano- 3- (trifluoromethoxy) phenoxy]-3- pyridyl]-2-methyl- propanamide (Intermediate 42) | LC/MS: QC_3_ MIN: Rt = 2.19 min; m/z 422 [M + H]⁺. | ¹H-NMR (500 MHz; DMSO-d6): δ ppm 8.37 (s, 1H), 8.06 (d, 1H), 7.52-7.48 (m, 1H), 7.43 (d, 1H), 7.34 (dd, 1H), 6.31 (bs, 2H), 6.27 (d, 1H), 1.55-1.23 (m, 6H). |
| 11 | 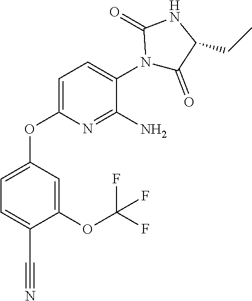 [SMILES: CC[C@H]1NC(=O)N(c2ccc(Oc3ccc(C#N)c(OC(F)(F)F)c3)nc2N)C1=O] | 4-[[6-amino-5- [(4R)-4-ethyl- 2,5-dioxo- imidazolidin-1- yl]-2- pyridyl]oxy]-2- (trifluoromethoxy) benzonitrile | (2R)-2-amino-N- [2-amino-6-[4- cyano-3- (trifluoromethoxy) phenoxy]-3- pyridyl] butanamide (Intermediate 43) | LC/MS: QC_3_ MIN: Rt = 2.53 min; m/z 422 [M + H]⁺. | ¹H-NMR (500 MHz; DMSO-d6): δ ppm 8.36 (bs, 1H), 8.05 (d, 1H), 7.50-7.48 (m, 1H), 7.38-7.30 (m, 2H), 6.50-6.19 (m, 3H), 4.11-3.95 (m, 1H), 1.85-1.53 (m, 2H), 1.01-0.81 (m, 3H). |
| 12 | 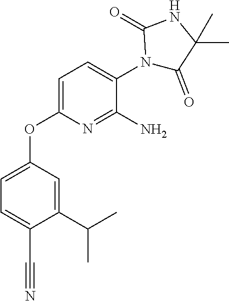 [SMILES: CC(C)c1cc(Oc2ccc(N3C(=O)NC(C)(C)C3=O)c(N)n2)ccc1C#N] | 4-[[6-amino-5- (4,4-dimethyl- 2,5-dioxo- imidazolidin-1- yl)-2- pyridyl]oxy]-2- isopropyl- benzonitrile | 2-amino-N-[2- amino-6-(4- cyano-3- isopropyl- phenoxy)-3- pyridyl]-2-methyl- propanamide (Intermediate 44) | LC/MS: QC_3_ MIN: Rt = 2.52 min; m/z 380 [M + H]⁺. | ¹H-NMR (500 MHz; DMSO-d6): δ ppm 8.39 (bs, 1H), 7.80 (d, 1H), 7.41 (d, 1H), 7.27 (d, 1H), 7.10 (dd, 1H), 6.30- 6.23 (m, 2H), 6.21 (d, 1H), 3.28-3.19 (m, 1H), 1.52-1.32 (m, 6H), 1.27 (d, 6H). |
| 13 | 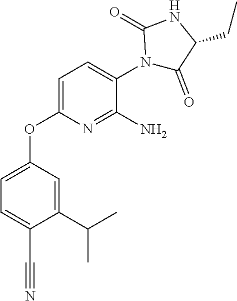 [SMILES: CC[C@H]1NC(=O)N(c2ccc(Oc3ccc(C#N)c(C(C)C)c3)nc2N)C1=O] | 4-[[6-amino-5- [(4R)-4-ethyl- 2,5-dioxo- imidazolidin-1- yl]-2- pyridyl]oxy]-2- isopropyl- benzonitrile | (2R)-2-amino-N- [2-amino-6-(4- cyano-3- isopropyl- phenoxy)-3- pyridyl] butanamide (Intermediate 45) | LC/MS: QC_3 MIN: Rt = 2.18 min; m/z 380 [M + H]⁺. | ¹H-NMR (500 MHz; DMSO-d6): δ ppm 8.38 (bs, 1H), 7.81 (d, 1H), 7.44-7.20 (m, 2H), 7.10 (dd, 1H), 6.54-6.01 (m, 3H), 4.22-3.97 (m, 1H), 3.30-3.20 (m, 1H), 1.89-1.60 (m, 2H), 1.28 (d, 6H), 1.02-0.88 (m, 3H). |
| 14 | 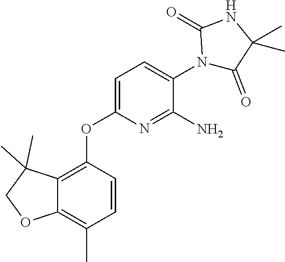 | 3-[2-amino-6- [(3,3,7-trimethyl- 2H-benzofuran- 4-yl)oxy]-3- pyridyl]-5,5- dimethyl- imidazolidine- 2,4-dione | 2-amino-N-[2- amino-6-[(3,3,7- trimethyl-2H- benzofuran-4- yl)oxy]-3-pyridyl]- 2-methyl- propanamide (Intermediate 46) | LC/MS: QC_3 MIN: Rt = 2.38 min; m/z 398 [M + H]⁺. | ¹H-NMR (500 MHz; DMSO-d6): δ ppm 8.34 (s, 1H), 7.26 (d, 1H), 6.92 (d, 1H), 6.41 (d, 1H), 6.11 (bs, 2H), 5.96 (d, 1H), 4.17 (s, 2H), 2.07 (s, 3H), 1.53-1.20 (m, 12H). |

### BIOLOGICAL EXAMPLES

### Biological Example 1: Measurement of Kv3.1, Kv3.2, Kv3.3 and Kv3.4 Channel Modulation

The ability of the compounds of the invention to modulate the voltage-gated potassium channel subtypes Kv3.3/Kv3.2/Kv3.1 may be determined using the following assay. Analogous methods may be used to investigate the ability of the compounds of the invention to modulate other channel subtypes.

### Cell Biology

To assess compound effects on human Kv3.3 channels (hKv3.3), a stable cell line expressing human Kv3.3 channels is created by transfecting Chinese Hamster Ovary (CHO)-K1 cells with a pBacMire_KCNC-3 vector. Cells are cultured in DMEM/F12 (Gibco) supplemented with 10% Foetal Bovine Serum (Gibco), 1× non-essential amino acids (Invitrogen) and geneticin (G418) 400 microg/mL. Cells are grown and maintained at 37° C. in a humidified environment containing 5% CO₂ in air.

To assess compound effects on human Kv3.2 channels (hKv3.2), a stable cell line expressing human Kv3.2 channels (hKv3.2) is created by transfecting CHO-K1 cells with a pCIH5-hKv3.2 vector. Cells are cultured in DMEM/F12 medium supplemented by 10% Foetal Bovine Serum, 1× non-essential amino acids (Invitrogen) and 500 ug/ml of Hygromycin-B (Invitrogen). Cells are grown and maintained at 37° C. in a humidified environment containing 5% CO₂ in air.

To assess compound effects on human Kv3.1 channels (hKv3.1): Human embryonic kidney (HEK)-hKv3.1 cell line is generated by transfecting HEK-293 cells with an expression vector with human Kv3.1 (NM_004976.4). Cells are cultured with MEM supplemented with 10% Heat-Inactivated FBS, 2 mM L-glutamine, 1% Penicillin-Streptomycin, and 0.6 mg/ml of Geneticin (G418). HEK-hKv3.1b cells were amplified in T175 cm2 flask at 37° C. with 5% C02, using MEM amplification medium, containing the G418 selection antibiotic (0.6 mg/ml). Cells were detached every 3-4 days, using DPBS to wash twice the flask, then TrypLE to dislodge the cells, and re-plated at a density of 2-4×106 cells/flask.

To assess compound effects on human Kv3.4 channels (hKv3.4): Human embryonic kidney (HEK)-hKv3.4 cell line is generated by transfecting HEK-293 cells with an expression vector with human Kv3.4 (NM_004978). Cells are cultured with MEM supplemented with 10% Heat-Inactivated FBS, 2 mM L-glutamine, 1% Penicillin-Streptomycin, and 0.6 mg/ml of Geneticin (G418). HEK-hKv3.4 cells were amplified in T175 cm2 flask at 37° C. with 5% C02, using MEM amplification medium, containing the G418 selection antibiotic (0.6 mg/ml). Cells were detached every 3-4 days, using DPBS to wash twice the flask, then TrypLE to dislodge the cells, and re-plated at a density of 4-8×106 cells/flask.

Cell preparation for IonWorks Quattro™ Experiments

The day of the experiment, cells are removed from the incubator and the culture medium removed. Cells are washed with 5 ml of Dulbecco's PBS (DPBS) calcium and magnesium free and detached by the addition of 3 ml Versene (Invitrogen, Italy) followed by a brief incubation at 37° C. for 5 minutes. The flask is tapped to dislodge cells and 10 ml of DPBS containing calcium and magnesium is added to prepare a cell suspension. The cell suspension is then placed into a 15 ml centrifuge tube and centrifuged for 2 min at 1200 rpm. After centrifugation, the supernatant is removed and the cell pellet re-suspended in 4 ml of DPBS containing calcium and magnesium using a 5 ml pipette to break up the pellet. Cell suspension volume is then corrected to give a cell concentration for the assay of approximately 3 million cells per ml.

All the solutions added to the cells are pre-warmed to 37° C.

### Electrophysiology

Experiments are conducted at r.t. using IonWorks Quattro™ planar array electrophysiology technology (Molecular Devices Corp.) with PatchPlate™ PPC. Stimulation protocols and data acquisition are carried out using a microcomputer (Dell Pentium 4). Planar electrode hole resistances (Rp) are determined by applying a 10 mV voltage step across each well. These measurements are performed before cell addition. After cell addition and seal formation, a seal test is performed by applying a voltage step from −80 mV to −70 mV for 160 ms. Following this, amphotericin-B solution is added to the intracellular face of the electrode to achieve intracellular access. Cells are held at −70 mV. Leak subtraction is conducted in all experiments by applying 50 ms hyperpolarizing (10 mV) prepulses to evoke leak currents followed by a 20 ms period at the holding potential before test pulses.

For hKv3.2 and hKv3.1, assays from the holding potential of −70 mV, a first test pulse at −15 mV was applied for 100 ms and after 100 ms at −70 mV a second pulse at +40 mV was applied for 50 ms. Cells were then maintained for 100 ms at −100 mV and another pulse from −70 mV to +40 mV (duration 50 ms) was applied to clamp later the voltage at −40 mV during 200 ms

For hKv3.3 assays, from the holding potential of −70 mV, a first test pulse to 0 mV is applied for 500 ms and following a further 100 ms at −70 mV, a second pulse to 40 mV is applied for 200 ms. These longer test pulses are used to study inactivation of hKv3.3 channels. Test pulses protocol may be performed in the absence (pre-read) and presence (post-read) of the test compound. Pre- and post-reads may be separated by the compound addition followed by a 3 minute incubation.

For hKv3.4, assays from the holding potential of −70 mV, a first test pulse at −15 mV was applied for 100 ms and after 200 ms at −70 mV a second pulse was applied at 0 mV for 100 ms then after 200 ms at −70 mV a third pulse was applied at +40 mV during 200 ms.

### Solutions and Drugs

The intracellular solution contains the following (in mM): K-gluconate 100, KCl 54, MgCl₂ 3.2, HEPES 5, adjusted to pH 7.3 with KOH. Amphotericin-B solution is prepared as 50 mg/ml stock solution in DMSO and diluted to a final working concentration of 0.1 mg/ml in intracellular solution. The external solution is Dulbecco's Phosphate Buffered Saline (DPBS) and contained the following (in mM): CaCl₂ 0.90, KCl 2.67, KH₂PO₄ 1.47, MgCl·6H₂O 0.493, NaCl 136.9, Na₃PO₄ 8.06, with a pH of 7.4.

Compounds of use in the invention (or reference compounds such as N-cyclohexyl-N-[(7,8-dimethyl-2-oxo-1,2-dihydro-3-quinolinyl)methyl]-N′-phenylurea) are dissolved in dimethylsulfoxide (DMSO) at a stock concentration of 10 mM. These solutions are further diluted with DMSO using a Biomek FX (Beckman Coulter) in a 384 compound plate. Each dilution (1 μL) is transferred to another compound plate and external solution containing 0.05% pluronic acid (66 μL) is added. 3.5 μL from each plate containing a compound of the invention is added and incubated with the cells during the IonWorks Quattro™ experiment. The final assay dilution is 200 and the final compound concentrations are in the range 50 μM to 50 nM.

### Data Analysis

The recordings are analysed and filtered using both seal resistance (>20 MΩ) and peak current amplitude (>500 pA at the voltage step of 40 mV) in the absence of compound to eliminate unsuitable cells from further analysis. For hKv3.2 and hKv3.1 assays, paired comparisons of evoked currents between pre- and post-drug additions measured for the −15 mV voltage step are used to determine the positive modulation effect of each compound. Kv3 channel-mediated outward currents are measured determined from the mean amplitude of the current over the final 10 ms of the −15 mV voltage pulse minus the mean baseline current at −70 mV over a 10 ms period just prior to the −15 mV step. These Kv3 channel currents following addition of the test compound are then compared with the currents recorded prior to compound addition. Data are normalised to the maximum effect of the reference compound (50 microM of N-cyclohexyl-N-[(7,8-dimethyl-2-oxo-1,2-dihydro-3-quinolinyl)methyl]-N′-phenylurea) and to the effect of a vehicle control (0.5% DMSO). The normalised data are analysed using ActivityBase or Excel software. The concentration of compound required to increase currents by 50% of the maximum increase produced by the reference compound (EC₅₀) is determined by fitting of the concentration-response data using a four parameter logistic function in ActivityBase. For hKv3.3 assays, paired comparisons of evoked currents between pre- and post-drug additions are measured for the 0 mV step, considering the peak current and the decay (inactivation) of the current over the duration of the 0 mv test pulse (500 ms).

N-cyclohexyl-N-[(7,8-dimethyl-2-oxo-1,2-dihydro-3-quinolinyl)methyl]-N′-phenylurea is obtained from ASINEX (Registry Number: 552311-06-5).

### Results

The results of certain Example compounds and their corresponding Reference Examples relating to the below generic formula are provided in Table 7. The Reference Examples have a central pyridyl ring which is unsubstituted or has a methyl or methoxy substitutent. Reference Examples 1 to 9 may be prepared using similar procedures to those disclosed herein, or by methods known to the skilled person.

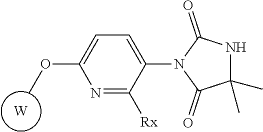

TABLE 7
Kv3.1 assay results

| Example | W | Rx | Kv3.1 pEC50 |
|---|---|---|---|
| Example 1 Reference Example 1 Reference Example 2 Reference Example 3 | 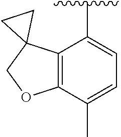 [SMILES: *c1ccc(C)c2c1C1(CC1)CO2] | NH₂ H OMe Me | 6.6 5.6 6.0 5.8 |
| Example 3 Reference Example 4 Reference Example 5 | 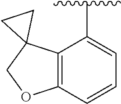 [SMILES: *c1cccc2c1C1(CC1)CO2] | NH₂ H OMe | 6.3 4.5 5.4 |
| Example 7 Reference Example 6 Reference Example 7 Reference Example 8 | 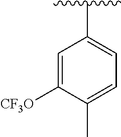 | NH₂ H OMe Me | 5.6 5.0 4.9 5.2 |
| Example 10 Reference Example 9 | 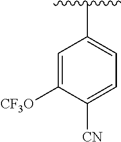 | NH₂ OMe | 5.6 4.9 |

The data in Table 7 demonstrate that the presence of an NH₂ group on the central pyridyl ring provides compounds with high pEC50 in a Kv3.1 assay. Removal of NH₂ substitution on the pyridyl ring leads to a decrease in potency. Replacement of NH₂ substitution on the pyridyl ring with OMe or Me, as shown in Table 7, leads to a decrease in potency.

The results of a number of Example compounds and their corresponding Reference Examples relating to the below generic formula are provided in Table 8. The Reference Examples have a central pyridyl ring which is unsubstituted or has a methyl or methoxy substitutent. Reference Examples 10 to 19 can be prepared using similar procedures to those disclosed herein, or by methods known to the skilled person.

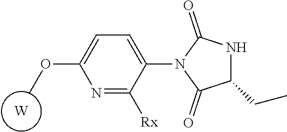

TABLE 8
Kv3.1 assay results

| Example | W | Rx | Kv3.1 pEC50 |
|---|---|---|---|
| Example 2 Reference Example 10 Reference Example 11 Reference Example 12 | 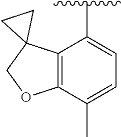 [SMILES: *c1ccc(C)c2c1C1(CC1)CO2] | NH₂ H OMe Me | 6.3 6.1 5.9 6.2 |
| Example 4 Reference Example 13 Reference Example 14 | 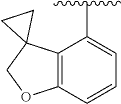 [SMILES: *c1cccc2c1C1(CC1)CO2] | NH₂ H OMe | 6.0 6.1 5.7 |
| Example 8 Reference Example 15 Reference Example 16 Reference Example 17 | 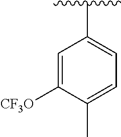 | NH₂ H OMe Me | 5.5 5.6 5.1 5.3 |
| Example 11 Reference Example 18 | 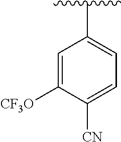 | NH₂ OMe | 5.5 5.0 |
| Example 13 Reference Example 19 | 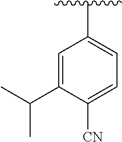 [SMILES: *c1ccc(C#N)c(C(C)C)c1] | NH₂ H | 5.5 5.9 |

The data in Table 8 demonstrate that the presence of an NH₂ group on the central pyridyl ring provides compounds with high pEC50 in a Kv3.1 assay. Replacement of NH₂ substitution on the pyridyl ring with OMe or Me, as shown in Table 8, leads to a decrease in potency.

The results of certain Example compounds and their corresponding Reference Examples relating to the below generic formula are provided in Table 9. The Reference Examples have a central pyridyl ring which bears an amine substituent at a different position to the corresponding Example compounds. Reference Examples 20 to 22 can be prepared using similar procedures to those disclosed herein, or by methods known to the skilled person.

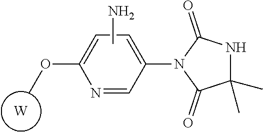

TABLE 9
Kv3.1 assay results

| Example | W | Amine | Kv3.1 pEC50 |
|---|---|---|---|
| Example 1 | 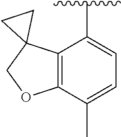 [SMILES: *c1ccc(C)c2c1C1(CC1)CO2] | 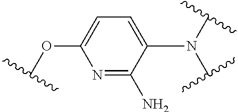 [SMILES: *Oc1ccc(N(*)*)c(N)n1] | 6.6 |
| Reference Example 20 | | 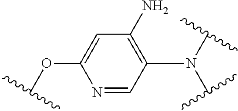 [SMILES: *Oc1cc(N)c(N(*)*)cn1] | 5.0 |
| Example 9 | 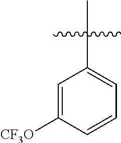 | 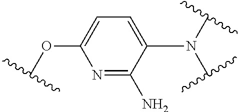 [SMILES: *Oc1ccc(N(*)*)c(N)n1] | 5.6 |
| Reference Example 21 | | 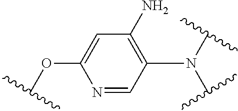 [SMILES: *Oc1cc(N)c(N(*)*)cn1] | 4.6 |
| Reference Example 22 | | 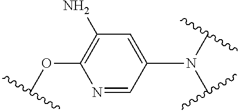 [SMILES: *Oc1ncc(N(*)*)cc1N] | 4.6 |

The data in Table 9 demonstrate that modification of the position of the NH₂ group on the central pyridyl ring, as shown in Table 9, can lead to a notable decrease in potency.

Data for all Example compounds when tested in the assay described above are presented in Table 10.

TABLE 10
Kv3.1 assay results

| Example Compound | Kv3.1 pEC₅₀ | Kv3.1 max R % |
|---|---|---|
| Example 1 | 6.6 | 183 |
| Example 2 | 6.3 | 161 |
| Example 3 | 6.3 | 159 |
| Example 4 | 6.0 | 159 |
| Example 5 | 6.2 | 152 |
| Example 6 | 4.9 | 89 |
| Example 7 | 5.6 | 135 |
| Example 8 | 5.5 | 137 |
| Example 9 | 5.6 | 118 |
| Example 10 | 5.6 | 113 |
| Example 11 | 5.5 | 161 |
| Example 12 | 5.7 | 175 |
| Example 13 | 5.5 | 161 |
| Example 14 | 6.3 | 165 |

The data in Table 10 show that compounds comprising a central pyridyl core substituted with a NH₂ group at a specific location display good pEC₅₀ properties in the Kv3.1 assay.

A secondary analysis of the data from the hKv3.1, hKv3.2 and hKv3.3 assays described in Biological Example 1 may be used to investigate the effect of the compounds on rate of rise of the current from the start of the depolarising voltage pulses. The magnitude of the effect of a compound can be determined from the time constant (Tau_{act}) obtained from a non-linear fit, using the equation given below, of the rise in Kv3.1, Kv3.2 and Kv3.3 currents following the start of the −15 mV depolarising voltage pulse.

$$Y = {( Y 0 - Y max )}^{*} exp ( - {K}^{*} X ) + Y max$$

where:
Y0 is the current value at the start of the depolarising voltage pulse;
Ymax is the plateau current;
K is the rate constant, and Tau_{act} is the activation time constant, which is the reciprocal of K.

Similarly, the effect of the compounds on the time taken for Kv3.1, Kv3.2 or Kv3.3 currents to decay on closing of the channels at the end of the −15 mV depolarising voltage pulses can also be investigated. In this latter case, the magnitude of the effect of a compound on channel closing can be determined from the time constant (Tau_{deact}) of a non-linear fit of the decay of the current (“tail current”) immediately following the end of the depolarising voltage pulse.

Kv3.1, Kv3.2 and Kv3.3 channels must activate and deactivate very rapidly in order to allow neurons to fire actions potentials at high frequency (Rudy et al., 2001). Slowing of activation is likely to delay the onset of action potential repolarisation; slowing of deactivation could lead to hyperpolarising currents that reduce the excitability of the neuron and delay the time before the neuron can fire a further action potential. Together these two slowing effects on channel activation and deactivation are likely to lead to a reduction rather than a facilitation of the neurons ability to fire at high frequencies. Thus compounds that have this slowing effect on the Kv3.1 and/or Kv3.2, and/or Kv3.3 channels will effectively behave as negative modulators of the channels, leading to a slowing of neuronal firing. This latter effect has been shown for certain of the compounds disclosed in WO2011/069951, where marked increases in Tau_{act} can be observed from recordings made from “fast-firing” interneurons in the cortex of rat brain, using electrophysiological techniques, in vitro. The addition of the relevant compounds reduces the ability of the neurons to fire in response to trains of depolarising pulses at 300 Hz.

Therefore, although certain compounds may be identified act as positive modulators in the recombinant cell assay of Biological Example 1, those compounds which markedly increase the value of Tauₐct can reduce the ability of neurons in native tissues to fire at high frequency.

### Biological Example 2: In Vitro Metabolic Stability Study in Human Hepatocytes

### Methodology

The objective of this study was to determine metabolic stability of test items in preclinical species and human liver microsomes. Verapamil and dextromethorphan were used as positive controls for microsomal incubation.

Incubation medium was prepared by combining 11.2 mL of potassium phosphate monobasic and 38.8 mL of potassium phosphate dibasic, diluted to 1 L with water. Cryopreserved microsomes were thawed and kept on ice until use. Microsomes were then diluted in incubation to a protein concentration of 0.56 mg/mL. Cells were centrifuged, re-suspended in medium and counted by means of a haemocytometer. Cell viability was measured using the Trypan Blue exclusion test.

NADPH regenerating system solution was prepared by dissolving 1.7 mg of NADP, 7.8 mg of G6P and 6 units of G6P-Dehydrogenase in 1 mL of 2% sodium bicarbonate solution (prepared by dissolving 20 g of NaHCO₃ in 1 L of water).

Test compounds were separately dissolved in DMF to obtain 10 mM stock solutions that were further diluted in water/acetonitrile 50/50 (v/v) to obtain the corresponding 50 uM working solutions. Verapamil and dextromethorphan were dissolved in DMF in order to obtain a 10 mM verapamil solution and 10 mM dextromethorphan solution. These solutions were then diluted in the incubation medium in order to obtain a 50 uM verapamil working solution and a 50 uM dextromethorphan working solution.

800 μL-aliquots of NADPH regenerating system were pre-warmed at 37° C. for 5 minutes. 5 μL of 50 μM test compounds, verapamil or dextromethorphan were added to 445 μL of the 0.56 mg/mL microsomes solution and the incubation mixture was pre-warmed in a 96 deep well 2 mL plate (incubation plate) at 37° C. for 5 minutes. The incubation reactions were initiated by adding 50 μL of pre-warmed NADPH regenerating system to the incubation mixtures. 50 μL aliquots were taken from incubation mixtures at: 0, 3, 6, 9, 15, and 30 minutes and the reactions were stopped by adding 100 μL of aceontrile with the corresponding internal standard. Samples were then diluted with 120 μL of water (final organic solvent percentage at 37%) and centrifuged at 3000 rpm for 10 minutes, prior the LC-MS/MS analysis.

Metabolic stability was calculated from the peak area ratio of the remaining test compound with internal standard versus time. The intrinsic clearance (CLint) was determined from the first order elimination constant k (min⁻¹) (obtained from GraphPad by plotting the natural logarithm of the peak area ratio of the remaining test item with internal standard versus time), using the actual volume of the incubation V (mL), the amount of hepatocytes in the incubation M (million cells) and the hepatocellularity number per g liver Hn (120 for human).

$$CLint = k * \frac{V}{M} * \frac{Hn \times {10}^{6} cells}{g liver}$$

Values for CLint were expressed as mL/min/g liver as shown in Table 5:

TABLE 11
CLint assay results

| | | CLi rat | CLi human |
|---|---|---|---|
| Example | Rx | (mL/min/g liver) | (mL/min/g liver) |
| Example 1 | NH₂ | 5.4 | 1.1 |
| Reference Example 2 | OMe | 8.7 | 2.0 |
| Example 9 | NH₂ | <0.5 | <0.5 |

The data in Table 11 show that compounds comprising a central pyridyl core substituted with a NH₂ group at a specific location (Example 1) can display lower clearance than those comprising an OMe substituent (corresponding Reference Example 2).

### Biological Example 3: CYP450 Inhibition Study

The method uses recombinant human CYP450 to metabolize pro-fluorescent probe substrates to fluorescent products. IC50 values for the effect of test compounds on the metabolism of these probe substrates provides a measure of the potency of inhibition of individual CYP450 isoforms.

The described assays measure in vitro inhibitory effects (pIC50) of compounds at the human P450 isoforms (1A2, 2C8, 2C9, 2C19, 2D6, 3A4) expressed in recombinant microsomes. Pro-fluorescent probe substrates are metabolized to fluorescent products by the enzymes.

Fluorescence is measured in kinetic mode (1 read/min for 10 min) and the fluorescence rate is calculated. Data are normalized to controls: DMSO represents 0% effect (i.e. no inhibition), while 10 μM Miconazole is set to 100% effect (i.e. complete inhibition).

Inhibition of the fluorescent signal determined by P450 activation indicates antagonist activity of the compound and allows for the calculation of the compound plC50.

A quality check (QC) is present in each compound plate using the following criteria:
The signal window is monitored by performing Z′ calculations (Z′≥0.2)
The antagonist potency of internal standards is within the pIC50±2SD range.

CRCs of compounds are run on two different test occasions (n=2) by preparing serial dilutions from the same compound stock solution. Details of the solutions are outlined in Table 6:

TABLE 12
Solution details

| Solutions | Components |
|---|---|
| Assay buffer | 100 mM Trizma HCl + 0.5 mM EDTA |
| Cofactor buffer | 2% (w/v) sodium bicarbonate |
| Cofactor | 7.8 mg glucose-6-phosphate, 1.7 mg NADP, 6 |
| | Units glucose-6-phosphate dehydrogenase/mL |
| | of 2% sodium bicarbonate |
| 1A2 Substrate | 50 μM ER (12 μg/ml in acetonitrile) |
| 2C9 Substrate | 12.5 mM FCA (3.76 mg/ml in acetonitrile) |
| 2C19 Substrate | 2.5 mM BMC (0.615 mg/ml in DMSO) |
| 2D6 Substrate | 2.5 mM MMMC (0.547 mg/ml in methanol) |
| 3A4 Substrate | 2.5 mM 7-BQ (0.588 mg/ml in acetonitrile) |

### Compound Plate Preparation

Test compound serial dilutions 1 to 3 were performed from a 10 mM stock solution in DMSO by Biomek FX to generate 10 point CRC. 100% DMSO was used for assay low controls. 10 mM (2 mM for 3A4/DBOMF, 2C8/DBOMF assay only) miconazole solution in DMSO was used as assay high controls. The final concentrations of the 10 point CRCs of test compounds in the assay plate were 5.00E-05; 1.67E-05; 5.56E-06; 1.85E-06; 6.17E-07; 2.06E-07; 6.86E-08; 2.29E-08; 7.62E-09 and 2.54E-09, respectively.

### Assay Plate Preparation

For each isoform assay buffer (14 ml), microsomes (330 μl at 0.1 mg/ml protein) and substrate (224 μl for 1A2/ER and 3A4HR/7BQ and 90 μl for 2C9/FCA, 2C19/BMC and 2D6/MMMC) were mixed together.

Microsomes were added to the buffer prior to the substrate addition. The solution was carefully mixed without vortexing. 30 μl/well of the mixture were then transferred to the assay plate (384-well black plate) using a16-channel pipette.

The CYP3A4 and CYP2C8 were assayed using the Vivid CYP3A4 Baculosomes, CYP2C8 Baculosomes and Vivid DBOMF Substrate.

Assay plate for CYP3A4 and CYP2C8 were prepared as follows:
Pre-mix was prepared by diluting P450 Baculosomes Plus Reagent and Vivid Regeneration System in 1× Vivid CYP450 Reaction Buffer (Potassium Phosphate Buffer, 100 mM, pH 8.0). A 8 μl/ml of P450 Baculosomes Plus Reagent and 16 μl/ml of Vivid Regeneration System were added to achieve the appropriate final concentrations (5 nM P450) and mixed by inversion. 30 μl of pre-mix was dispendsed into each well of a black, 384-well plate.

10 μl/well from compound plate was added and incubated for 10 min at 37° C. on a plate shaker. A mixture of Vivid Substrate (5 μl/ml for 3A4/DBOMF and 2.5 μl/ml for 2C8/DBOMF) Vivid NADP+ (50 μl/ml) in Vivid CYP450 Reaction Buffer was prepared. The reaction was started by adding 10 μl/well of the Vivid Substrate and NADP+ mixture. The plate was transferred into the fluorescent plate reader (EnVision, Wallac) immediately within 2 min and monitor fluorescence every minute for 10 (Excitation 485, Emission 535 and Mirror 505 FITC). A separate plate for each P450 isoform was prepared.

### Fluorescence Measurement

10 μl/well of the test compounds were transferred from compound plate to assay plate using Biomek FX with the appropriate protocol. The assay plate was then incubated at 37° C. for 10 min on a shaker to allow for the interaction between compounds and enzymes.

After that 10 μl/well of the cofactor was added to the assay plate using the Multidrop before placing the assay plate in the plate reader EnVision to measure fluorescence.

The plate was read every minute for 10 min according to the P450 isoform specific protocol. The overall final DMSO concentration for the assay was 0.5%.

The inhibition effect was expressed as percentage. Normalization was based on the inhibition rate in the presence of 10 μM Miconazole (100% effect=C2) and inhibition rate in presence of 0.5% DMSO (0% effect=C1), according to the following formula:

Y=100×[(data−C1)/(C2−C1)]

Curve fitting and pIC50 estimations are carried out using a four-parameter logistic model in Morphit.

The results of the assay are shown in Table 13:

TABLE 13
CYP450 assay results

| Example | Rx | CYP1A2 | CYP2C19 | CYP2C8 | CYP2C9 | CYP2D6 | CYP3A47BQ | CYP3A4DBOMF |
|---|---|---|---|---|---|---|---|---|
| Example 1 | NH₂ | >50 | >50 | 8.9 | 8.6 | >50 | >50 | >50 |
| Reference | OMe | >50 | 1.1 | 2.3 | 0.1 | >50 | >50 | 10.2 |
| Example 2 | | | | | | | | |
| Example 9 | NH₂ | >50 | 21 | >50 | >50 | >50 | >50 | >50 |

The data presented in Table 13 show that compounds comprising a central pyridyl core substituted with a NH₂ group at a specific location (Example 1) are less potent inhibitors of CYP450 isoforms than those comprising an OMe substituent (corresponding Reference Example 2).

### Additional Animal Models

Patent applications WO2011/069951, WO2012/076877, WO2012/168710, WO2013/175215 WO2013/182851, WO2013/083994, WO2013/182850, WO2017/103604, WO2018/020263, WO2018/109484 and WO2020/079422 (all incorporated by reference for the purpose of demonstrate the activity of compounds which are modulators of Kv3.1 and Kv3.2) demonstrate the activity of compounds which are modulators of Kv3.1 and Kv3.2 in animal models of seizure, hyperactivity, sleep disorders, psychosis, hearing disorders, bipolar disorders and pain.

Patent application WO2013/175211 (incorporated by reference for the purpose of demonstrate the activity of compounds which are modulators of Kv3.1 and Kv3.2) demonstrates the efficacy of a compound which is a modulator of Kv3.1 and Kv3.2 in a model of acute noise-induced hearing loss in the chinchilla, and also evaluates the efficacy of the compound in a model of central auditory processing deficit and in a model of tinnitus.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims which follow.

The invention is further illustrated by way of the following clauses:

### Clauses of the Invention

Clause 1. A compound of formula (I):

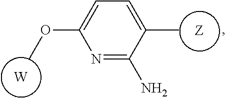

wherein
W is group (Wa), group (Wb) or group (Wc):
   wherein group (Wa) and group (Wb) are:

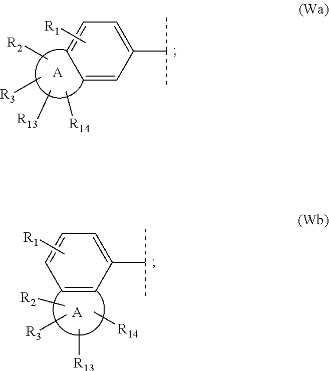

wherein:
   R₁ is H, C₁₋₄alkyl, halo, haloC₁₋₄alkyl, CN, C₁₋₄alkoxy or haloC₁₋₄alkoxy;
   R₂ is H, C₁₋₄alkyl, C₃₋₅ spiro carbocyclyl, haloC₁₋₄alkyl or halo;
   R₃ is H, C₁₋₄alkyl, haloC₁₋₄alkyl, halo; or R₃ is absent;
   R₁₃ is H, C₁₋₄alkyl, haloC₁₋₄alkyl, halo; or R₁₃ is absent;
   R₁₄ is H, C₁₋₄alkyl, haloC₁₋₄alkyl, halo; or R₁₄ is absent;
   A is a 5 or 6 membered saturated or unsaturated heterocycle, with at least one O atom; which heterocycle is optionally fused with a cyclopropyl group, or a cyclobutyl group, or a cyclopentyl group to form a tricycle when considered together with the phenyl;
   wherein R₂ and R₃ may be attached to the same or a different ring atom; R₂ may be attached to a fused ring atom; and wherein R₁₃ and R₁₄ may be attached to the same or a different ring atom;
wherein group (Wc) is:

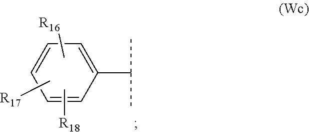

wherein:
   R₁₆ is halo, C₁₋₄alkyl, C₁₋₄alkoxy, haloC₁₋₄alkyl, haloC₁₋₄alkoxy or CN;
   R₁₇ is H, halo, CN, C₁₋₄alkyl, C₁₋₄alkoxy or haloC₁₋₄alkoxy;
   R₁₈ is H, halo, CN, C₁₋₄alkyl or C₁₋₄alkoxy;
Z is group (Za):

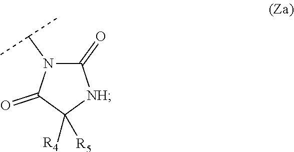

wherein:
R₄ is H or C₁₋₄ alkyl;
R₅ is H or C₁₋₄ alkyl; or
R₄ and R₅ can be fused to form a C₃₋₅ spiro carbocyclyl;
or a salt and/or solvate and/or derivative thereof.

Clause 2. The salt and solvate according to clause 1.

Clause 3. The salt according to clause 1.

Clause 4. The solvate according to clause 1.

Clause 5. The derivative according to clause 1.

Clause 6. The compound, pharmaceutically acceptable salt and/or solvate and/or derivative thereof according to clause 1.

Clause 7. The compound, pharmaceutically acceptable salt and/or solvate thereof according to clause 6.

Clause 8 The pharmaceutically acceptable salt and solvate according to clause 6.

Clause 9. The pharmaceutically acceptable solvate according to clause 6.

Clause 10. The pharmaceutically acceptable salt according to clause 6.

Clause 11. The compound according to clause 6.

Clause 12 The compound, salt thereof and/or solvate thereof and/or derivative thereof according to any one of clauses 1 to 11, wherein R₄ is methyl, ethyl, isopropyl or tert-butyl.

Clause 13. The compound, salt thereof and/or solvate thereof and/or derivative thereof according to clause 12, wherein R₄ is methyl.

Clause 14. The compound, salt thereof and/or solvate thereof and/or derivative thereof according to any one of clauses 1 to 13, wherein R₅ is H.

Clause 15. The compound, salt thereof and/or solvate thereof and/or derivative thereof according to any one of clauses 1 to 13, wherein one of R₄ and R₅ is methyl and the remaining R₄ or R₅ is ethyl.

Clause 16. The compound, salt thereof and/or solvate thereof and/or derivative thereof according to any one of clauses 1 to 15, wherein R₄ and R₅ are different and have the stereochemical arrangement:

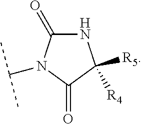

Clause 17. The compound, salt thereof and/or solvate thereof and/or derivative thereof according to any one of clauses 1 to 13, wherein R₄ and R₅ are both methyl or both ethyl.

Clause 18. The compound, salt thereof and/or solvate thereof and/or derivative thereof according to clause 16, wherein R₄ and R₅ are both methyl.

Clause 19. The compound, salt thereof, solvate thereof and/or derivative thereof according to any one of clauses 1 to 12, wherein R₄ is H and R₅ is H.

Clause 20. The compound, salt thereof and/or solvate thereof and/or derivative thereof according to any one of clauses 1 to 12, wherein R₄ and R₅ are fused to form a C₃₋₄ spiro carbocyclyl.

Clause 21. The compound, salt thereof and/or solvate thereof and/or derivative thereof according to any one of clauses 1 to 12 or 16, wherein R₄ is C₁₋₄ alkyl such as methyl, ethyl, isopropyl or tert-butyl, and R₅ is H.

Clause 22. The compound, salt thereof and/or solvate thereof and/or derivative thereof according to clause 21 or 16, wherein R₄ is ethyl and R₅ is H.

Clause 23. The compound, salt thereof and/or solvate thereof and/or derivative thereof according to any one of clauses 1 to 22, wherein W is group (Wa).

Clause 24. The compound, salt thereof and/or solvate thereof and/or derivative thereof according to any one of clauses 1 to 23, wherein W is group (Wb).

Clause 25. The compound, salt thereof and/or solvate thereof and/or derivative thereof according to clause 24 wherein (Wb) is:

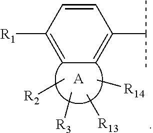

Clause 26. The compound, salt thereof and/or solvate thereof and/or derivative thereof according to any one of clauses 1 to 25, wherein R₁ is H, C₁₋₄alkyl, halo, haloC₁₋₄alkyl or CN.

Clause 27. The compound, salt thereof and/or solvate thereof and/or derivative thereof according to clause 26, wherein R₁ is H, methyl or CN.

Clause 28. The compound, salt thereof and/or solvate thereof and/or derivative thereof according to any one of clauses 1 to 25, wherein R₁ is C₁₋₄alkoxy such as methoxy.

Clause 29. The compound, salt thereof and/or solvate thereof and/or derivative thereof according to any one of clauses 1 to 28, wherein ring A is selected from the group consisting of:

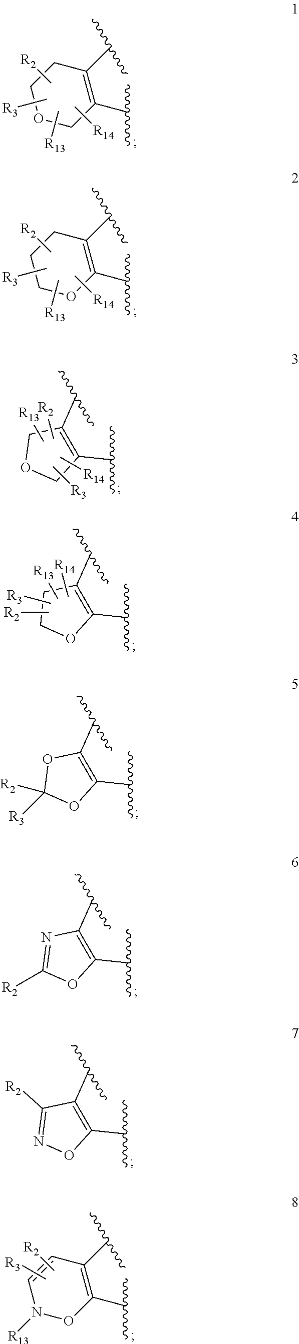

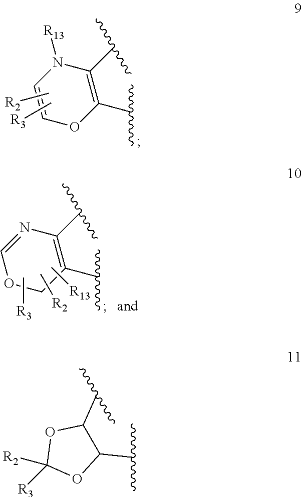

wherein

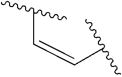 [SMILES: */C=C\*]

denotes a point at which ring A is fused to the phenyl ring.

Clause 30. The compound, salt thereof and/or solvate thereof and/or derivative thereof according to clause 29, wherein ring A is selected from the group consisting of:

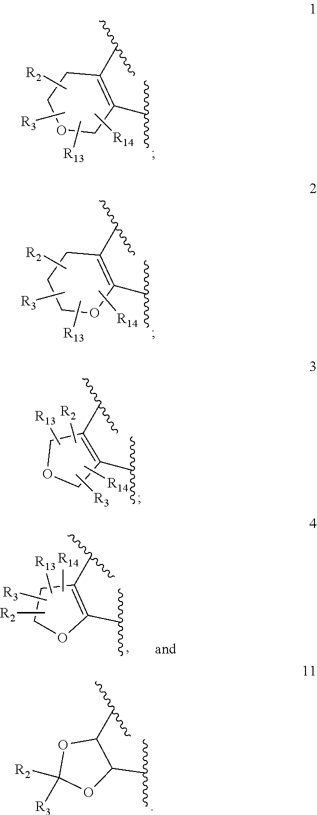

Clause 31. The compound, salt thereof and/or solvate thereof and/or derivative thereof according to clause 30, wherein ring A is:

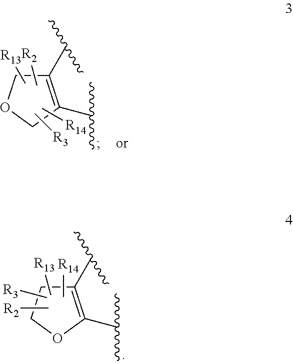

Clause 32. The compound, salt thereof and/or solvate thereof and/or derivative thereof according to any one of clauses 1 to 31, wherein R₂ is H, C₁₋₄alkyl, C₃₋₅spiro carbocyclyl or halo; in particular C₁₋₄ alkyl or C₃₋₅spiro carbocyclyl.

Clause 33. The compound, salt thereof and/or solvate thereof and/or derivative thereof according to clause 32, wherein R₂ is C₃ spiro carbocyclyl.

Clause 34. The compound, salt thereof and/or solvate thereof and/or derivative thereof according to clause 32, wherein R₂ is methyl or halo e.g. fluoro.

Clause 35. The compound, salt thereof and/or solvate thereof and/or derivative thereof according to clause 34, wherein R₂ is methyl.

Clause 36 The compound, salt thereof and/or solvate thereof and/or derivative thereof according to any one of clauses 1 to 35, wherein R₃ is H, C₁₋₄alkyl, haloC₁₋₄alkyl or halo.

Clause 37. The compound, salt thereof and/or solvate thereof and/or derivative thereof according to clause 36, wherein R₃ is methyl or halo e.g. fluoro.

Clause 38. The compound, salt thereof and/or solvate thereof and/or derivative thereof according to clause 37, wherein R₃ is methyl.

Clause 39. The compound, salt thereof and/or solvate thereof and/or derivative thereof according to any one of clauses 1 to 38, wherein R₁₃ is H or is absent and is suitably absent.

Clause 40. The compound, salt thereof and/or solvate thereof and/or derivative thereof according to any one of clauses 1 to 39, wherein R₁₄ is H or is absent and is suitably absent.

Clause 41. The compound, salt thereof and/or solvate thereof and/or derivative thereof according to any one of clauses 1 to 22, wherein W is:

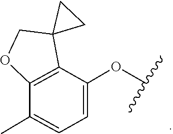 [SMILES: *Oc1ccc(C)c2c1C1(CC1)CO2]

Clause 42. The compound, salt thereof and/or solvate thereof and/or derivative thereof according to any one of clauses 1 to 22, wherein W is

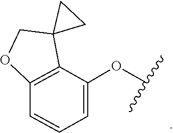 [SMILES: *Oc1cccc2c1C1(CC1)CO2]

Clause 43. The compound, salt thereof and/or solvate thereof and/or derivative thereof according to any one of clauses 1 to 22, wherein W is:

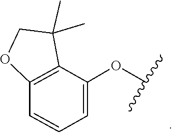 [SMILES: *Oc1cccc2c1C(C)(C)CO2]

Clause 44. The compound, salt thereof and/or solvate thereof and/or derivative thereof according to any one of clauses 1 to 22, wherein W is:

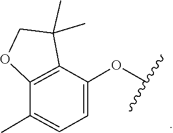 [SMILES: *Oc1ccc(C)c2c1C(C)(C)CO2]

Clause 45. The compound, salt thereof and/or solvate thereof and/or derivative thereof according to any one of clauses 1 to 22, wherein W is group (Wc).

Clause 46. The compound, salt thereof and/or solvate thereof and/or derivative thereof according to any one of clauses 1 to 22 or 45, wherein R₁₆ is C₁₋₄alkyl, C₁₋₄alkoxy, haloC₁₋₄alkyl, haloC₁₋₄alkoxy or CN.

Clause 47. The compound, salt thereof and/or solvate thereof and/or derivative thereof according to clause 46, wherein R₁₆ is methyl, ethyl, propyl, butyl, cyclopropyl, chloro, fluoro, methoxy, ethoxy, propoxy, trifluoromethyl, trifluoromethoxy or CN.

Clause 48. The compound, salt thereof and/or solvate thereof and/or derivative thereof according to clause 47, wherein R₁₆ is methoxy, trifluoromethoxy or tert-butyl.

Clause 49. The compound, salt thereof and/or solvate thereof and/or derivative thereof according to clause 47 or 48, wherein R₁₆ is at the ortho- or meta-position.

Clause 50. The compound, salt thereof and/or solvate thereof and/or derivative thereof according to any one of clauses 1 to 22 or 45 to 49, wherein R₁₇ is H, halo, CN, C₁₋₄alkyl or C₁₋₄alkoxy.

Clause 51. The compound, salt thereof and/or solvate thereof and/or derivative thereof according to clause 50, wherein R₁₇ is H, methyl, ethyl, propyl, butyl, cyclopropyl, chloro, fluoro, methoxy, ethoxy, propoxy, trifluoromethoxy or CN.

Clause 52. The compound, salt thereof and/or solvate thereof and/or derivative thereof according to clause 51, wherein R₁₇ is H, methyl or CN.

Clause 53. The compound, salt thereof and/or solvate thereof and/or derivative thereof according to any one of clauses 1 to 22 or 45 to 49, wherein R₁₇ is at the para-position.

Clause 54. The compound, salt thereof and/or solvate thereof and/or derivative thereof according to any one of clauses 1 to 22 or 45 to 53, wherein R₁₈ is H.

Clause 55. The compound, salt thereof and/or solvate thereof and/or derivative thereof according to any one of clauses 1 to 22, wherein W is:

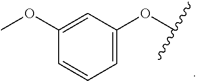 [SMILES: *Oc1cccc(OC)c1]

Clause 56. The compound, salt thereof and/or solvate thereof and/or derivative thereof according to any one of clauses 1 to 22, wherein W is:

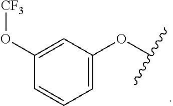 [SMILES: *Oc1cccc(OC(F)(F)F)c1]

Clause 57. The compound, salt thereof and/or solvate thereof and/or derivative thereof according to any one of clauses 1 to 22, wherein W is:

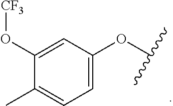 [SMILES: *Oc1ccc(C)c(OC(F)(F)F)c1]

Clause 58. The compound, salt thereof and/or solvate thereof and/or derivative thereof according to any one of clauses 1 to 22, wherein W is:

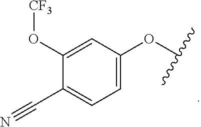 [SMILES: *Oc1ccc(C#N)c(OC(F)(F)F)c1]

Clause 59. The compound, salt thereof and/or solvate thereof and/or derivative thereof according to any one of clauses 1 to 22, wherein W is:

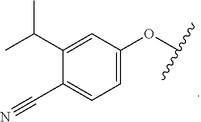 [SMILES: *Oc1ccc(C#N)c(C(C)C)c1]

Clause 60. The compound, pharmaceutically acceptable salt thereof and/or solvate thereof and/or derivative thereof according to clause 1, wherein the compound of formula (I) is selected from the group consisting of:
3-[2-amino-6-(7-methylspiro[2H-benzofuran-3,1-cyclopropane]-4-yl)oxy-3-pyridyl]-5,5-dimethyl-imidazolidine-2,4-dione;
(5R)-3-[2-amino-6-(7-methylspiro[2H-benzofuran-3,1′-cyclopropane]-4-yl)oxy-3-pyridyl]-5-ethyl-imidazolidine-2,4-dione;
3-(2-amino-6-spiro[2H-benzofuran-3,1′-cyclopropane]-4-yloxy-3-pyridyl)-5,5-dimethyl-imidazolidine-2,4-dione;
(5R)-3-(2-amino-6-spiro[2H-benzofuran-3,1′-cyclopropane]-4-yloxy-3-pyridyl)-5-ethyl-imidazolidine-2,4-dione;
3-[2-amino-6-[(3,3-dimethyl-2H-benzofuran-4-yl)oxy]-3-pyridyl]-5,5-dimethyl-imidazolidine-2,4-dione;
3-[2-amino-6-(3-methoxyphenoxy)-3-pyridyl]-5,5-dimethyl-imidazolidine-2,4-dione;
3-[2-amino-6-[4-methyl-3-(trifluoromethoxy)phenoxy]-3-pyridyl]-5,5-dimethyl-imidazolidine-2,4-dione;
(5R)-3-[2-amino-6-[4-methyl-3-(trifluoromethoxy)phenoxy]-3-pyridyl]-5-ethyl-imidazolidine-2,4-dione;
3-[2-amino-6-[3-(trifluoromethoxy)phenoxy]-3-pyridyl]-5,5-dimethyl-imidazolidine-2,4-dione;
4-[[6-amino-5-(4,4-dimethyl-2,5-dioxo-imidazolidin-1-yl)-2-pyridyl]oxy]-2-(trifluoromethoxy)benzonitrile;
4-[[6-amino-5-[(4R)-4-ethyl-2,5-dioxo-imidazolidin-1-yl]-2-pyridyl]oxy]-2-(trifluoromethoxy)benzonitrile;
4-[[6-amino-5-(4,4-dimethyl-2,5-dioxo-imidazolidin-1-yl)-2-pyridyl]oxy]-2-isopropyl-benzonitrile; 4-[[6-amino-5-[(4R)-4-ethyl-2,5-dioxo-imidazolidin-1-yl]-2-pyridyl]oxy]-2-isopropyl-benzonitrile; and
3-[2-amino-6-[(3,3,7-trimethyl-2H-benzofuran-4-yl)oxy]-3-pyridyl]-5,5-dimethyl-imidazolidine-2,4-dione.

Clause 61. The compound, salt thereof and/or solvate thereof and/or derivative thereof according to clause 1, which is 3-[2-amino-6-(7-methylspiro[2H-benzofuran-3,1′-cyclopropane]-4-yl)oxy-3-pyridyl]-5,5-dimethyl-imidazolidine-2,4-dione:

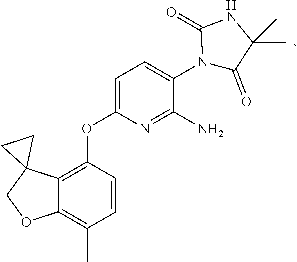 [SMILES: Cc1ccc(Oc2ccc(N3C(=O)NC(C)(C)C3=O)c(N)n2)c2c1OCC21CC1]

such as a pharmaceutically acceptable salt thereof and/or solvate thereof and/or derivative thereof.

Clause 62. The compound, salt thereof and/or solvate thereof and/or derivative thereof according to clause 1, wherein the compound of formula (I) is 3-[2-amino-6-[3-(trifluoromethoxy)phenoxy]-3-pyridyl]-5,5-dimethyl-imidazolidine-2,4-dione:

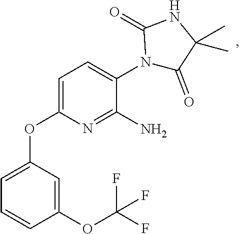 [SMILES: CC1(C)NC(=O)N(c2ccc(Oc3cccc(OC(F)(F)F)c3)nc2N)C1=O]

such as a pharmaceutically acceptable salt thereof and/or solvate thereof and/or derivative thereof.

Clause 63. The compound according to any one of clauses 60 to 62, wherein the compound is in the form of a pharmaceutically acceptable salt.

Clause 64. The compound according to any one of clauses 60 to 62, wherein the compound is not in the form of a salt.

Clause 65. A pharmaceutical composition comprising the compound, pharmaceutically acceptable salt thereof and/or solvate thereof, and/or derivative thereof according to any one of clauses 1 to 64 and a pharmaceutically acceptable carrier or excipient.

Clause 66. The compound, pharmaceutically acceptable salt thereof and/or solvate thereof, and/or derivative thereof according to any one of clauses 1 to 64 or the pharmaceutical composition according to clause 65 for use as a medicament.

Clause 67. The compound, pharmaceutically acceptable salt thereof and/or solvate thereof, and/or derivative thereof according to any one of clauses 1 to 64 or the pharmaceutical composition for use according to clause 65 for use in the prophylaxis or treatment of a disease or disorder selected from the group consisting of hearing disorders, schizophrenia, depression and mood disorders, bipolar disorder, substance abuse disorders, anxiety disorders, sleep disorders, hyperacusis and disturbances of loudness perception, Meniere's disease, disorders of balance, and disorders of the inner ear, impulse control disorder, personality disorders, attention-deficit/hyperactivity disorder, autism spectrum disorders, eating disorders, cognition impairment, ataxia, pain such as neuropathic pain, inflammatory pain and miscellaneous pain, Lewy body dementia and Parkinson's disease.

Clause 68. The compound, pharmaceutically acceptable salt thereof and/or solvate thereof, and/or derivative thereof according to any one of clauses 1 to 64 or the pharmaceutical composition for use according to clause 65 for use in the prophylaxis or treatment of progressive myoclonic epilepsy, including PME associated with mutations in the KCNC1 gene, hearing disorders, including hearing loss and tinnitus, Fragile X syndrome, schizophrenia, substance abuse disorders, or pain.

Clause 69. Use of a compound, pharmaceutically acceptable salt thereof and/or solvate thereof, and/or derivative thereof according to any one of clauses 1 to 64 or the pharmaceutical composition for use according to clause 65 in the manufacture of a medicament.

Clause 70. Use of according to clause 69, in the manufacture of a medicament for the prophylaxis or treatment of progressive myoclonic epilepsy, including PME associated with mutations in the KCNC1 gene, hearing disorders, including hearing loss and tinnitus, Fragile X syndrome, schizophrenia, substance abuse disorders, or pain.

Clause 71. A method for the prophylaxis or treatment of progressive myoclonic epilepsy, including PME associated with mutations in the KCNC1 gene, hearing disorders, including hearing loss and tinnitus, Fragile X syndrome, schizophrenia, substance abuse disorders, or pain, said method comprising administering a compound, pharmaceutically acceptable salt thereof and/or solvate thereof, and/or derivative thereof according to any one of clauses 1 to 64 or the pharmaceutical composition for use according to clause 65 to a subject.

Clause 72. The compound, pharmaceutically acceptable salt thereof and/or solvate thereof, and/or derivative thereof, the pharmaceutical composition, use or method according to according to any one of clauses 1 to 71, for use in the prophylaxis or treatment of progressive myoclonic epilepsy.

Clause 73. The compound, pharmaceutically acceptable salt thereof and/or solvate thereof, and/or derivative thereof, the pharmaceutical composition, use or method according to according to any one of clauses 1 to 71, for use in the prophylaxis or treatment of hearing loss.

Clause 74. The compound, pharmaceutically acceptable salt thereof and/or solvate thereof, and/or derivative thereof, the pharmaceutical composition, use or method according to according to any one of clauses 1 to 71, for use in the prophylaxis or treatment of tinnitus.

Clause 75. The compound, pharmaceutically acceptable salt thereof and/or solvate thereof, and/or derivative thereof, the pharmaceutical composition, use or method according to according to any one of clauses 1 to 71, for use in the prophylaxis or treatment of Fragile X syndrome.

Clause 76. The compound, pharmaceutically acceptable salt thereof and/or solvate thereof, and/or derivative thereof, the pharmaceutical composition, use or method according to according to any one of clauses 1 to 71, for use in the prophylaxis or treatment of schizophrenia.

Clause 77. The compound, pharmaceutically acceptable salt thereof and/or solvate thereof, and/or derivative thereof, the pharmaceutical composition, use or method according to according to any one of clauses 1 to 71, for use in the prophylaxis or treatment of substance abuse disorders.

Clause 78. The compound, pharmaceutically acceptable salt thereof and/or solvate thereof, and/or derivative thereof, the pharmaceutical composition, use or method according to according to any one of clauses 1 to 71, for use in the prophylaxis or treatment of pain, such as neuropathic pain, inflammatory pain or miscellaneous pain.

Clause 79. The compound, pharmaceutically acceptable salt thereof and/or solvate thereof, and/or derivative thereof, the pharmaceutical composition, use or method according to according to any one of clauses 1 to 71, for use in the prophylaxis or treatment of cardiovascular disease.

Clause 80. The compound, pharmaceutically acceptable salt thereof and/or solvate thereof, and/or derivative thereof, the pharmaceutical composition, use or method according to according to any one of clauses 1 to 71, for use in the prophylaxis or treatment of muscular disorders such as Spinal-Bulbar Muscular Atrophy (SBMA).

Clause 81. The compound, pharmaceutically acceptable salt thereof and/or solvate thereof, and/or derivative thereof, the pharmaceutical composition, use or method according to according to clause 80, for use in the prophylaxis or treatment of Spinal-Bulbar Muscular Atrophy (SBMA).

Clause 82. The compound, pharmaceutically acceptable salt thereof and/or solvate thereof, and/or derivative thereof, the pharmaceutical composition, use or method according to according to any one of clauses 1 to 81, for use in prophylaxis.

Clause 83. The compound, pharmaceutically acceptable salt thereof and/or solvate thereof, and/or derivative thereof, the pharmaceutical composition, use or method according to according to any one of clauses 1 to 82, for use in treatment.

Clause 84. The derivative according to any one of clauses 1 to 64, functionalised via the secondary nitrogen of the hydantoin or via the secondary nitrogen of the triazolone with a group L as illustrated directly above, wherein L is selected from:
a) —PO(OH)O⁻·M⁺, wherein M+ is a pharmaceutically acceptable monovalent counterion,
b) —PO(O⁻)₂·2M⁺,
c) —PO(O⁻)₂·D²⁺, wherein D²⁺ is a pharmaceutically acceptable divalent counterion,
d) —CH(R^{X})—PO(OH)O⁻·M⁺, wherein R^{X} is hydrogen or C₁₋₃ alkyl,
e) —CH(R^{X})—PO(O⁻)₂·2M⁺,
f) —CH(R^{X})—PO(O⁻)₂·D²⁺,
g) —SO₃⁻·M⁺,
h) —CH(R^{X})—SO₃⁻·M⁺, and
i) —CO—CH₂CH₂—CO₂·M⁺.

Clause 85. A process for the preparation of a compound of formula (I), or a salt and/or solvate and/or derivative thereof, which comprises reacting a compound of formula (II):

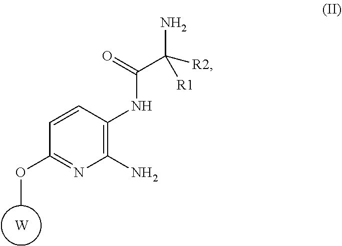

or a salt thereof, wherein W, R₁ and R₂ are as defined for the compound of formula (I), with a carbonylating agent e.g. triphosgene.

Clause 86. A process for the preparation of a compound of formula (II), or a salt thereof, which comprises reacting a compound of formula (III):

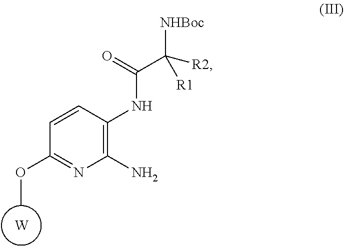

or a salt thereof, wherein W, R₁ and R₂ are as defined for the compound of formula (I), with an acid e.g. trifluoroacetic acid.

Clause 87. A process for the preparation of a compound of formula (III), or a salt thereof, which comprises reacting a compound of formula (IV),

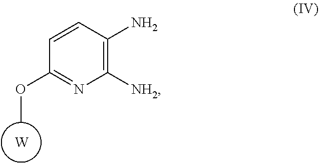

or a salt thereof, wherein W is as defined for the compound of formula (I), with a compound of formula (V):

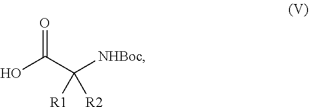

or a salt thereof, wherein R₁ and R₂ are as defined for the compound of formula (I), in the presence of a coupling agent e.g. HATU and a base e.g. DIPEA.

Clause 88. A process for the preparation of a compound of formula (IV), or a salt thereof, which comprises reacting a compound of formula (VI):

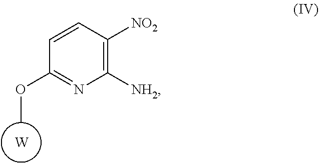

or a salt thereof, under reduction conditions e.g. in the presence of Fe powder and ammonium chloride.

Clause 89. A process for the preparation of a compound of formula (VI), or a salt and/or solvate and/or derivative thereof, which comprises reacting a compound of formula (VII):

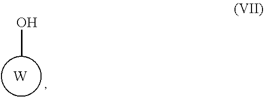

or a salt thereof, wherein W is as defined for the compound of formula (I), with a compound of formula (VIII):

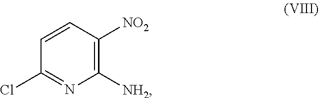 [SMILES: Nc1nc(Cl)ccc1[N+](=O)[O-]]

or a salt thereof.

Clause 90. A compound or salt thereof selected from the group consisting of:
a compound of formula (II):

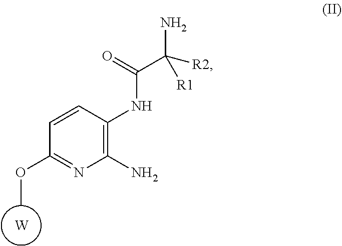

wherein W, R₁ and R₂ are as defined for the compound of formula (I); and
a compound of formula (III):

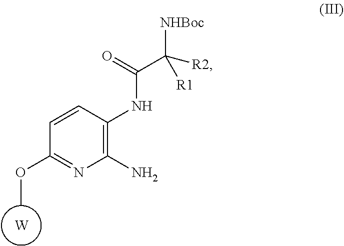

wherein W, R₁ and R₂ are as defined for the compound of formula (I)
a compound of formula (IV):

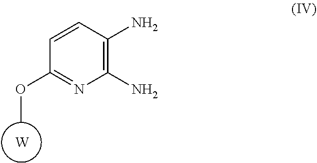

wherein W is as defined for the compound of formula (I)
a compound of formula (VI):

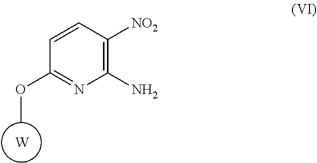

wherein W is as defined for the compound of formula (I).

Clause 91: The compound, salt and/or solvate and/or derivative thereof according to any preceeding claim, which is in natural isotopic form.

Clause 92: The compound, pharmaceutically acceptable salt thereof and/or solvate thereof and/or derivative thereof according to any preceeding claim, wherein the pharmaceutically acceptable salt thereof and/or solvate thereof and/or derivative thereof is a pharmaceutically acceptable salt thereof, solvate thereof and/or derivative thereof.

Clause 93. The compound 3-[2-amino-6-(7-methylspiro[2H-benzofuran-3,1′-cyclopropane]-4-yl)oxy-3-pyridyl]-5,5-dimethyl-imidazolidine-2,4-dione:

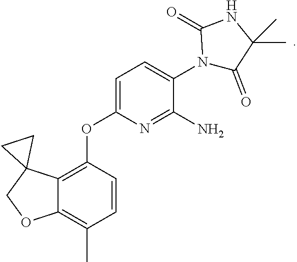 [SMILES: Cc1ccc(Oc2ccc(N3C(=O)NC(C)(C)C3=O)c(N)n2)c2c1OCC21CC1]

Clause 94. The compound 3-[2-amino-6-[3-(trifluoromethoxy)phenoxy]-3-pyridyl]-5,5-dimethyl-imidazolidine-2,4-dione:

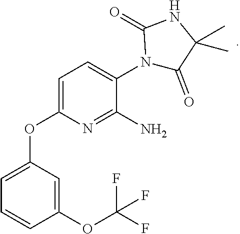 [SMILES: CC1(C)NC(=O)N(c2ccc(Oc3cccc(OC(F)(F)F)c3)nc2N)C1=O]

### REFERENCES

All publications, including but not limited to patents and patent applications, cited in this specification are herein incorporated by reference as if each individual publication were specifically and individually indicated to be incorporated by reference herein as though fully set forth.
Anderson L A et al. Increased spontaneous firing rates in auditory midbrain following noise exposure are specifically abolished by a Kv3 channel modulator. Hear Res. 2018 August; 365:77-89
Andrade-Talavera et al., J. Physiol. (2020) 598, 3711-3725
Aroniadou-Anderjaska V et al. Mechanisms regulating GABAergic inhibitory transmission in the basolateral amygdala: implications for epilepsy and anxiety disorders. Amino Acids 2007 August; 32:305-315.
Baranauskas G, Nistri A. Sensitization of pain pathways in the spinal cord: cellular mechanisms. Prog. Neurobiol. 1998 February; 54(3):349-65.
Baron R et al. Peripheral input and its importance for central sensitization. Ann. Neurol. 2013 November; 74(5):630-6.
Ben-Ari Y. Seizure Beget Seizure: The Quest for GABA as a Key Player. Crit. Rev. Neurobiol. 2006; 18(1-2):135-144.
Benes F M et al. Circuitry-based gene expression profiles in GABA cells of the trisynaptic pathway in schizophrenics versus bipolars. PNAS 2008 December; 105(52):20935-20940.
Bennett D L, Woods C G. Painful and painless channelopathies. Lancet Neurol. 2014 June; 13(6):587-99.
Berge S et al. Pharmaceutical Salts. J. Pharm. Sci. 1977; 66; 1-19.
Boddum et al., Neuropharm. (2017) 118, 102-112
Brambilla P et al. GABAergic dysfunction in mood disorders. Mol. Psych. 2003 April; 8:721-737.
Brooke R E et al. Spinal cord interneurones labelled transneuronally from the adrenal gland by a GFP-herpes virus construct contain the potassium channel subunit Kv3.1b. Auton. Neurosci. 2002 June; 98(1-2):45-50.
Brooke R E et al. Association of potassium channel Kv3.4 subunits with pre- and post-synaptic structures in brainstem and spinal cord. Neuroscience 2004; 126(4):1001-10.
Brooke R E et al. Immunohistochemical localisation of the voltage gated potassium ion channel subunit Kv3.3 in the rat medulla oblongata and thoracic spinal cord. Brain Res. 2006 January; 1070(1):101-15.
Cervero F. Spinal cord hyperexcitability and its role in pain and hyperalgesia. Exp. Brain Res. 2009 June; 196(1):129-37.
Chambers A R et al. Pharmacological modulation of Kv3.1 mitigates auditory midbrain temporal processing deficits following auditory nerve damage. Sci Rep. 2017 Dec. 13; 7(1):17496
Chang S Y et al. Distribution of Kv3.3 Potassium Channel Subunits in Distinct Neuronal Populations of Mouse Brain. J. Comp. Neuro. 2007 February; 502:953-972.
Chien L Y et al. Reduced expression of A-type potassium channels in primary sensory neurons induces mechanical hypersensitivity. J. Neurosci. 2007 September; 27(37):9855-65.
Chow A et al. K⁺ Channel Expression Distinguishes Subpopulations of Parvalbumin- and Somatostatin-Containing Neocortical Interneurons. J. Neurosci. 1999 November; 19(21):9332-9345.
Darnell et al., Cell 2001, 107, 489-499
Desai R et at. Protein Kinase C Modulates Inactivation of Kv3.3 Channels. J. Biol. Chem. 2008; 283; 22283-22294.
Deuchars S A et al. Properties of interneurones in the intermediolateral cell column of the rat spinal cord: role of the potassium channel subunit Kv3.1. Neuroscience 2001; 106(2):433-46.
Devulder J. Flupirtine in pain management: pharmacological properties and clinical use. CNS Drugs 2010 October; 24(10):867-81.
Dib-Hajj S D et al. The Na(V)1.7 sodium channel: from molecule to man. Nat. Rev. Neurosci. 2013 January; 14(1):49-62.
Diochot S et al. Sea Anemone Peptides with a Specific Blocking Activity against the Fast Inactivating Potassium Channel Kv3.4. J. Biol. Chem. 1998 March; 273(12); 6744-6749.
   Engel A K et al. Dynamic Predictions: Oscillations and Synchrony in Top-Down Processing. Nat. Rev. Neurosci. 2001 October; 2(10):704-716.
EI-Hassar et al., J Neurosci. 2019 39, 4797-4813
Espinosa F et al. Alcohol Hypersensitivity, Increased Locomotion, and Spontaneous Myoclonus in Mice Lacking the Potassium Channels Kv3.1 and Kv3.3. J. Neurosci. 2001 September; 21(17):6657-6665.
Espinosa F et al. Ablation of Kv3.1 and Kv3.3 Potassium Channels Disrupts Thalamocortical Oscillations In Vitro and In Vivo. J. Neurosci. 2008 May; 28(21):5570-5581.
Figueroa K et al. KCNC3: phenotype, mutations, channel biophysics—a study of 260 familial ataxia patients. Human Mutation. 2010; 31; 191-196.
Finnerup N B et at. Pharmacotherapy for neuropathic pain in adults: a systematic review and meta-analysis. Lancet Neurol. 2015 February; 14(2):162-73.
Fisahn A. Kainate receptors and rhythmic activity in neuronal networks: hippocampal gamma oscillations as a tool. J. Physiol. 2005 October; 561(1):65-72.
Glait L et al. Effects of AUT00063, a Kv3.1 channel modulator, on noise-induced hyperactivity in the dorsal cochlear nucleus. Hear Res. 2018 April; 361:36-44
Greene T W, Wuts, PG. Greene's Protective Groups in Organic Synthesis, 2006, 4th Edition, John Wiley & Sons, Inc., Hoboken, NJ, USA.
Joho R H et at. Increased γ- and Decreased 6-Oscillations in a Mouse Deficient for a Potassium Channel Expressed in Fast-Spiking Interneurons. J. Neurophysiol. 1999 June; 82:1855-1864.
Joho R H, Hurlock E C. The Role of Kv3-type Potassium Channels in Cerebellar Physiology and Behavior. Cerebellum 2009 February; 8:323-333.
Jung D et al. Age-related changes in the distribution of Kv1.1 and Kv3.1 in rat cochlear nuclei. Neurol. Res. 2005; 27; 436-440.
Kasten M R et al. Differential regulation of action potential firing in adult murine thalamocortical neurons by Kv3.2, Kv1, and SK potassium and N-type calcium channels. J. Physiol. 2007; 584(2):565-582.
Kaczmarek L et al. Regulation of the timing of MNTB neurons by short-term and long-term modulation of potassium channels. Hearing Res. 2005; 206; 133-145.
Lau D et al. Impaired Fast-Spiking, Suppressed Cortical Inhibition, and Increased Susceptibility to Seizures in Mice Lacking Kv3.2 K⁺ Channel Proteins. J. Neurosci. 2000 December; 20(24):9071-9085.
Leger et al., European Neuropsychopharmacology Volume 25, Supplement 2, September 2015, Page S480
Li W et al. Localization of Two High-Threshol Potassium Channel Subunits in the Rat Central Auditory System. J. Comp. Neuro. 2001 May; 437:196-218.
Lu R et al. Slack channels expressed in sensory neurons control neuropathic pain in mice. J. Neurosci. 2015 January; 35(3):1125-35.
Markram H et al. Interneurons of the neocortical inhibitory system. Nat. Rev. Neurosci. 2004 October; 5:793-807.
Martina M et al. Functional and Molecular Differences between Voltage-Gated K⁺ Channels of Fast-Spiking Interneurons and Pyramidal Neurons of Rat Hippocampus. J. Neurosci. 1998 October; 18(20):8111-8125.
McCarberg B H et al. The impact of pain on quality of life and the unmet needs of pain management: results from pain sufferers and physicians participating in an Internet survey. Am. J. Ther. 2008 July-August; 15(4):312-20.
McDonald A J, Mascagni F. Differential expression of Kv3.1b and Kv3.2 potassium channel subunits in interneurons of the basolateral amygdala. Neuroscience 2006; 138:537-547.
McMahon A et al. Allele-dependent changes of olivocerebellar circuit properties in the absence of the voltage-gated potassium channels Kv3.1 and Kv3.3. Eur. J. Neurosci. 2004 March; 19:3317-3327.
Muona M, et al. A recurrent de novo mutation in KCNC1 causes progressive myoclonus epilepsy.

### Nat Genet. 2015 January; 47(1):39-46.

Muqeem T et al. Regulation of Nociceptive Glutamatergic Signaling by Presynaptic Kv3.4 Channels in the Rat Spinal Dorsal Horn J Neurosci. 2018 Apr. 11; 38(15):3729-3740
Olsen T et al. Kv3 K+ currents contribute to spike-timing in dorsal cochlear nucleus principal cells. Neuropharmacology 2018 May 1:133:319-333
Parekh et al., Neuropsychopharmacology volume 43, pages 435-444 (2018)
Pilati N et al., Acoustic over-exposure triggers burst firing in dorsal cochlear nucleus fusiform cells. Hearing Research 2012; 283; 98-106.
Pirbhoy et al., Journal of Neurochemistry 2020 Volume 155, Issue 5 Pages 538-558
Puente N et al. Precise localization of the voltage-gated potassium channel subunits Kv3.1b and Kv3.3 revealed in the molecular layer of the rat cerebellar cortex by a pre-embedding immunogold method. Histochem. Cell. Biol. 2010 September; 134:403-409.
Reynolds G P et al. Calcium Binding Protein Markers of GABA Deficits in Schizophrenia—Post Mortem Studies and Animal Models. Neurotox. Res. 2004 February; 6(1):57-62.
Ritter D M et al. Modulation of Kv3.4 channel N-type inactivation by protein kinase C shapes the action potential in dorsal root ganglion neurons. J. Physiol. 2012 January; 590(Pt 1):145-61.
Ritter D M et al. Dysregulation of Kv3.4 channels in dorsal root ganglia following spinal cord injury. J. Neurosci. 2015 January; 35(3):1260-73.
Roberts L et al. Ringing Ears: The Neuroscience of Tinnitus. J. Neurosci. 2010:30(45); 14972-14979.
Rudy B, McBain C J. Kv3 channels: voltage-gated K⁺ channels designed for high-frequency repetitive firing. TRENDS in Neurosci. 2001 September; 24(9):517-526.
Sacco T et al. Properties and expression of Kv3 channels in cerebellar Purkinje cells. Mol. Cell. Neurosci. 2006 July; 33:170-179.
Schulz P, Steimer T. Neurobiology of Circadian Systems. CNS Drugs 2009; 23(Suppl. 2):3-13.
Song P et al. Acoustic environment determines phosphorylation state of the Kv3.1 potassium channel in auditory neurons Nat. Neurosci. 2005 October; 8(10): 1335-1342.
Spencer K M et al. Neural synchrony indexes disordered perception and cognition in schizophrenia. PNAS 2004 December; 101(49):17288-17293.
Strumbos et al., J Neurosci. 2010 30, 10263-10271
Sun S et al. Inhibitors of voltage-gated sodium channel Nav1.7: patent applications since 2010. Pharm. Pat. Anal. 2014 September; 3(5):509-21.
U.S. Department of Health and Human Services, Food and Drug Administration. Draft Guidance for Industry Analgesic Indications: Developing Drug and Biological Products: http://www.fda.gov/downloads/drugs/Quidancecomplianceregulatorvinformation/guidances/ucm384691.pdf 2014 February
von Hehn C et al. Loss of Kv3.1 Tonotopicity and Alterations in cAMP Response Element-Binding Protein Signaling in Central Auditory Neurons of Hearing Impaired Mice. J. Neurosci. 2004; 24: 1936-1940.
Wickenden A D, McNaughton-Smith G. Kv7 channels as targets for the treatment of pain. Curr. Pharm. Des. 2009; 15(15):1773-98.
Woolf C J. What is this thing called pain? J. Clin. Invest. 2010 November; 120(11):3742-4.
Woolf C J. Central sensitization: implications for the diagnosis and treatment of pain. Pain 2011 March; 152(3 Suppl): S2-15.
Yanagi M et al. Kv3.1-containing K(+) channels are reduced in untreated schizophrenia and normalized with antipsychotic drugs. Mol Psychiatry. 2014. 19(5):573-9.
Yeung S Y M et al. Modulation of Kv3 Subfamily Potassium Currents by the Sea Anemone Toxin BDS: Significance for CNS and Biophysical Studies. J. Neurosci. 2005 March; 25(38):8735-8745.
Zamponi G W et al. The Physiology, Pathology, and Pharmacology of Voltage-Gated Calcium Channels and Their Future Therapeutic Potential Pharmacol Rev. 2015 October; 67(4):821-70.

1. A compound of formula (I): 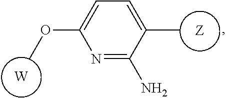
wherein
W is group (Wa), group (Wb) or group (Wc):
wherein group (Wa) and group (Wb) are: 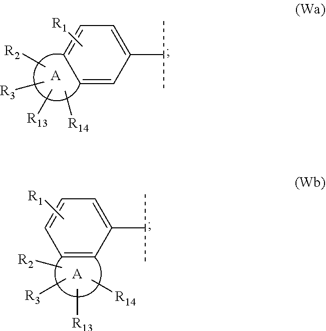
wherein:
R₁ is H, C₁₋₄alkyl, halo, haloC₁₋₄alkyl, CN, C₁₋₄alkoxy or haloC₁₋₄alkoxy;
R₂ is H, C₁₋₄alkyl, C₃₋₅ spiro carbocyclyl, haloC₁₋₄alkyl or halo;
R₃ is H, C₁₋₄alkyl, haloC₁₋₄alkyl, halo; or R₃ is absent;
R₁₃ is H, C₁₋₄alkyl, haloC₁₋₄alkyl, halo; or R₁₃ is absent;
R₁₄ is H, C₁₋₄alkyl, haloC₁₋₄alkyl, halo; or R₁₄ is absent;
A is a 5 or 6 membered saturated or unsaturated heterocycle, with at least one O atom; which heterocycle is optionally fused with a cyclopropyl group, or a cyclobutyl group, or a cyclopentyl group to form a tricycle when considered together with the phenyl;
wherein R₂ and R₃ may be attached to the same or a different ring atom; R₂ may be attached to a fused ring atom; and wherein R₁₃ and R₁₄ may be attached to the same or a different ring atom;
wherein group (We) is: 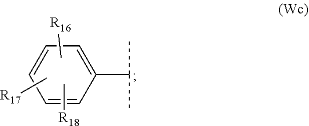
wherein:
R₁₆ is halo, C₁₋₄alkyl, C₁₋₄alkoxy, haloC₁₋₄alkyl, haloC₁₋₄alkoxy or CN;
R₁₇ is H, halo, CN, C₁₋₄alkyl, C₁₋₄alkoxy or haloC₁₋₄alkoxy;
R₁₈ is H, halo, CN, C₁₋₄alkyl or C₁₋₄alkoxy;
Z is group (Za): 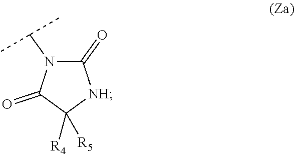
wherein:
R₄ is H or C₁₋₄ alkyl;
R₅ is H or C₁₋₄ alkyl; or
R₄ and R₅ can be fused to form a C₃₋₅ spiro carbocyclyl;
or a salt and/or solvate and/or derivative thereof.

2. The compound of formula (I) according to claim 1 or a pharmaceutically acceptable salt and/or solvate and/or derivative thereof.

3. The compound of formula (I) according to claim 2 or a pharmaceutically acceptable salt and/or solvate thereof.

4. The compound, pharmaceutically acceptable salt thereof and/or solvate thereof and/or derivative thereof according to claim 1, wherein R₄ is ethyl and R₅ is H.

5. The compound, pharmaceutically acceptable salt thereof and/or solvate thereof and/or derivative thereof according to claim 1, wherein R₄ and R₅ are both methyl.

6. The compound, pharmaceutically acceptable salt thereof and/or solvate thereof and/or derivative thereof according to claim 1, wherein W is group (Wb):

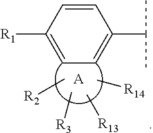

7. The compound, pharmaceutically acceptable salt thereof and/or solvate thereof and/or derivative thereof according to claim 1, wherein R₁ is H, methyl or CN.

8. The compound, pharmaceutically acceptable salt thereof and/or solvate thereof and/or derivative thereof according to claim 1, wherein ring A is selected from the group consisting of: 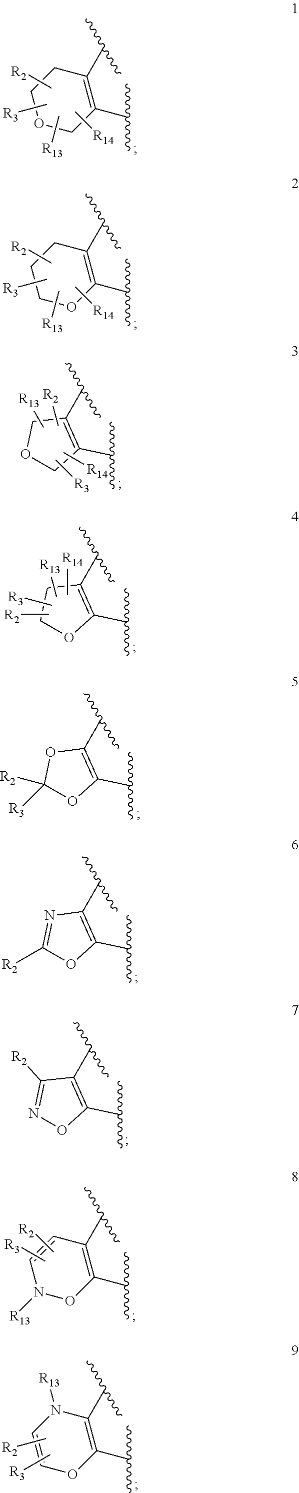 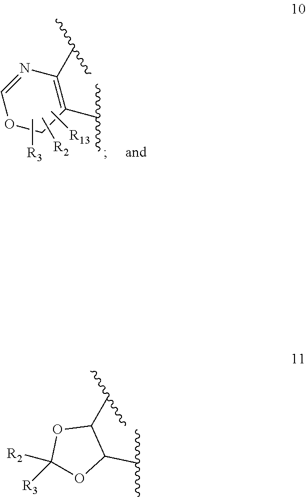
optionally wherein ring, A is selected from the group consisting of 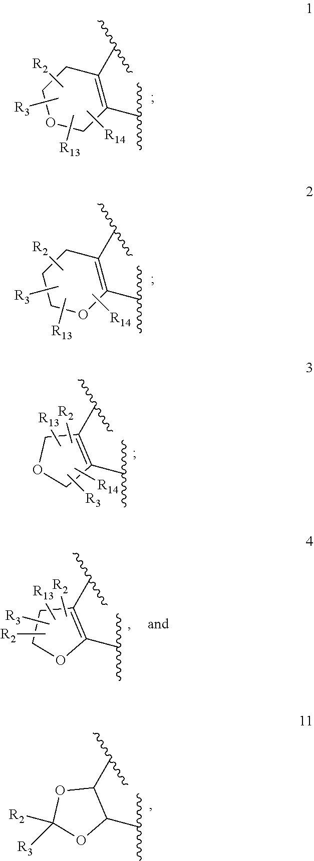
or wherein ring, A is 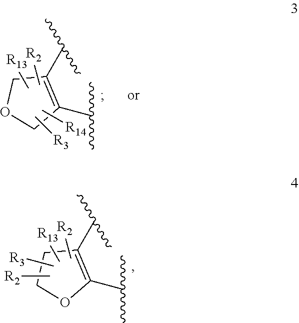
wherein 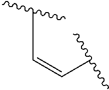 [SMILES: */C=C\*]
denotes a point at which ring A is fused to the phenyl ring.

9. The compound, pharmaceutically acceptable salt thereof and/or solvate thereof and/or derivative thereof according to claim 1, wherein R₂ is C₃ spiro carbocyclyl.

10. The compound, pharmaceutically acceptable salt thereof and/or solvate thereof and/or derivative thereof according to claim 1, wherein R₂ is methyl or halo e.g. fluoro.

11. The compound, pharmaceutically acceptable salt thereof and/or solvate thereof and/or derivative thereof according to claim 1, wherein R₂ is methyl and wherein R₃ is methyl.

12. The compound, pharmaceutically acceptable salt thereof and/or solvate thereof and/or derivative thereof according to claim 1, wherein W is selected from the group consisting of

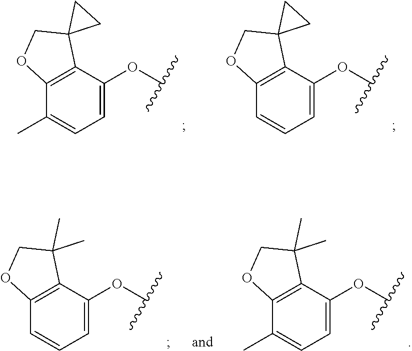 [SMILES: *Oc1ccc(C)c2c1C1(CC1)CO2 ; *Oc1cccc2c1C1(CC1)CO2 ; *Oc1cccc2c1C(C)(C)CO2 ; *Oc1ccc(C)c2c1C(C)(C)CO2]

13. The compound, pharmaceutically acceptable salt thereof and/or solvate thereof and/or derivative thereof according to claim 1, wherein W is group (Wc).

14. The compound, pharmaceutically acceptable salt thereof and/or solvate thereof and/or derivative thereof according to claim 13, wherein:
R₁₆ is methyl, ethyl, propyl, butyl, cyclopropyl, chloro, fluoro, methoxy, ethoxy, propoxy, trifluoromethyl, trifluoromethoxy or CN, optionally wherein R₁₆ is methoxy, trifluoromethoxy or tert-butyl; and
R₁₇ is H, methyl, ethyl, propyl, butyl, cyclopropyl, chloro, fluoro, methoxy, ethoxy, propoxy, trifluoromethoxy or CN, optionally wherein R₁₇ is H, methyl or CN.

15. The compound, pharmaceutically acceptable salt thereof and/or solvate thereof and/or derivative thereof according to claim 13, wherein W is selected from the group consisting of

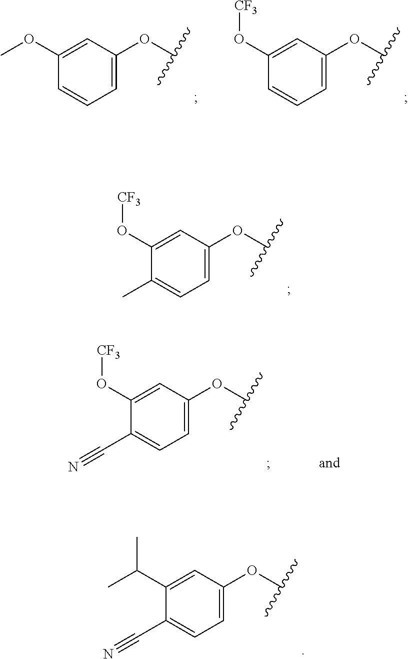

16. The compound, pharmaceutically acceptable salt thereof and/or solvate thereof and/or derivative thereof according to claim 2, selected from the group consisting of:
3-[2-amino-6-(7-methylspiro[2H-benzofuran-3,1′-cyclopropane]-4-yl)oxy-3-pyridyl]-5,5-dimethyl-imidazolidine-2,4-dione;
(5R)-3-[2-amino-6-(7-methylspiro[2H-benzofuran-3,1′-cyclopropane]-4-yl)oxy-3-pyridyl]-5-ethyl-imidazolidine-2,4-dione;
3-(2-amino-6-spiro[2H-benzofuran-3,1′-cyclopropane]-4-yloxy-3-pyridyl)-5,5-dimethyl-imidazolidine-2,4-dione;
(5R)-3-(2-amino-6-spiro[2H-benzofuran-3,1′-cyclopropane]-4-yloxy-3-pyridyl)-5-ethyl-imidazolidine-2,4-dione;
3-[2-amino-6-[(3,3-dimethyl-2H-benzofuran-4-yl)oxy]-3-pyridyl]-5,5-dimethyl-imidazolidine-2,4-dione;
3-[2-amino-6-(3-methoxyphenoxy)-3-pyridyl]-5,5-dimethyl-imidazolidine-2,4-dione;
3-[2-amino-6-[4-methyl-3-(trifluoromethoxy)phenoxy]-3-pyridyl]-5,5-dimethyl-imidazolidine-2,4-dione;
(5R)-3-[2-amino-6-[4-methyl-3-(trifluoromethoxy)phenoxy]-3-pyridyl]-5-ethyl-imidazolidine-2,4-dione;
3-[2-amino-6-[3-(trifluoromethoxy)phenoxy]-3-pyridyl]-5,5-dimethyl-imidazolidine-2,4-dione;
4-[[6-amino-5-(4,4-dimethyl-2,5-dioxo-imidazolidin-1-yl)-2-pyridyl]oxy]-2-(trifluoromethoxy)benzonitrile;
4-[[6-amino-5-[(4R)-4-ethyl-2,5-dioxo-imidazolidin-1-yl]-2-pyridyl]oxy]-2-(trifluoromethoxy)benzonitrile;
4-[[6-amino-5-(4,4-dimethyl-2,5-dioxo-imidazolidin-1-yl)-2-pyridyl]oxy]-2-isopropyl-benzonitrile;
4-[[6-amino-5-[(4R)-4-ethyl-2,5-dioxo-imidazolidin-1-yl]-2-pyridyl]oxy]-2-isopropyl-benzonitrile; and
3-[2-amino-6-[(3,3,7-trimethyl-2H-benzofuran-4-yl)oxy]-3-pyridyl]-5,5-dimethyl-imidazolidine-2,4-dione;
or a pharmaceutically acceptable salt and/or solvate and/or derivative thereof.

17. The compound 3-[2-amino-6-(7-methylspiro[2H-benzofuran-3,1′-cyclopropane]-4-yl)oxy-3-pyridyl]-5,5-dimethyl-imidazolidine-2,4-dione:

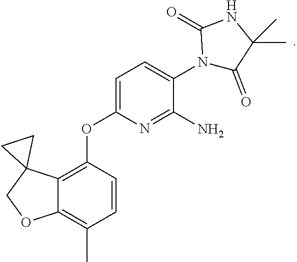 [SMILES: Cc1ccc(Oc2ccc(N3C(=O)NC(C)(C)C3=O)c(N)n2)c2c1OCC21CC1]

18. The compound 3-[2-amino-6-[3-(trifluoromethoxy)phenoxy]-3-pyridyl]-5,5-dimethyl-imidazolidine-2,4-dione:

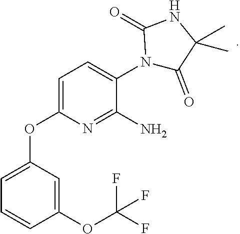 [SMILES: CC1(C)NC(=O)N(c2ccc(Oc3cccc(OC(F)(F)F)c3)nc2N)C1=O]

19-22. (canceled)

23. A method for the prophylaxis or treatment of progressive myoclonic epilepsy, including PME associated with mutations in the KCNC1 gene, hearing disorders, including hearing loss and tinnitus, Fragile X syndrome, schizophrenia, substance abuse disorders, or pain, said method comprising administering a compound, pharmaceutically acceptable salt thereof and/or solvate thereof, and/or derivative thereof according to claim 2 to a subject.

24. A compound or salt thereof selected from the group consisting of:
a compound of formula (II): 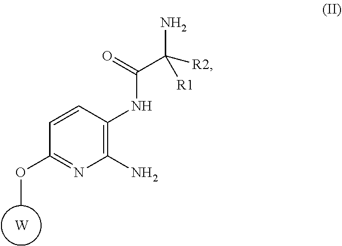
wherein W, R₁ and R₂ are as defined for the compound of formula (I); or
a compound of formula (III): 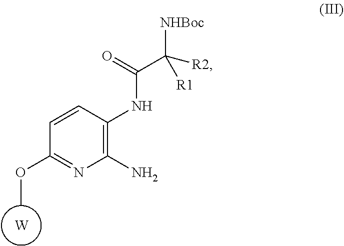
wherein W, R₁ and R₂ are as defined for the compound of formula (I),
a compound of formula (IV): 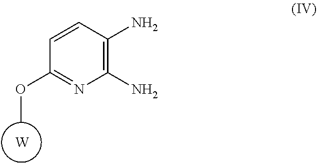
wherein W is as defined for the compound of formula (I)
a compound of formula (VI): 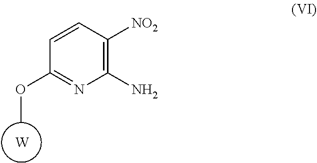
wherein W is as defined for the compound of formula (I).
